(19) United States
(12) Patent Application Publication
(10) Pub. No.: US 2011/0262556 A1
(43) Pub. Date: Oct. 27, 2011

(54) SILVER/WATER, SILVER GELS AND SILVER-BASED COMPOSITIONS; AND METHODS FOR MAKING AND USING THE SAME

(75) Inventors: Robert J. Holladay, Saratoga, UT (US); William Moeller, Alpine, UT (US); Dillip Mehta, Mumbai (IN); Rustum Roy, State College, PA (US); Julian H.J. Brooks, North East, MD (US); Mark G. Mortenson, North East, MD (US)

(73) Assignee: AMERICAN SILVER, LLC, Alpine, UT (US)

(21) Appl. No.: 11/813,408

(22) PCT Filed: Dec. 30, 2005
(86) PCT No.: PCT/US05/47699

§ 371 Date: Apr. 27, 2011

Related U.S. Application Data
(60) Provisional application No. 60/641,521, filed on Jan. 5, 2005.
(60) Provisional application No. 60/697,079, filed on Jul. 7, 2005.
(60) Provisional application No. 60/702,494, filed on Jul. 26, 2005.

(51) Int. Cl. A01N 59/16; A01N 59/20; A61K 33/24; A61K 33/30; A61K 33/34; A61P 17/00; A61K 31/65; A61K 31/555; A01P 1/00; A61P 31/04; A61P 31/10; A61K 33/38; A61K 33/40

(52) U.S. Cl. 424/616; 424/618; 514/495; 424/630; 424/641; 424/649; 424/617; 514/152; 514/188

(57) ABSTRACT

We disclose a colorless composition comprising metal particles (e.g., silver nanoparticles) and water, wherein said particles comprise an interior of elemental metal (e.g., silver) and an exterior of metal oxide (e.g., one or more silver oxide(s)), wherein the metal nanoparticles are present in the water at a level of about 5-40 ppm, and wherein the composition manifests significant antimicrobial properties. Methods of use of the composition are described. The composition can be incorporated into a hydrogel with essentially no loss of antimicrobial properties. Various metal-containing compositions with unexpected biological efficacy are also disclosed.

### AREA OF THE ART

The present invention generally relates to novel silver/water mixtures (sometimes referred to as silver nanoparticles dispersed in water), and more particularly to novel compositions and/or morphologies of silver/water mixtures, silver hydrogels, novel silver compositions combined with modern antibiotics and various ligands bounded to silver ions, silver gels based upon certain starting silver/water mixtures, silver ions and/or metal(s) bonded to/contained in certain clathrates such as clays and/or zeolite materials, and to methods for making and using said compositions as agents against various organisms (including certain viruses) harmful to the health or wellness of humans and/or animals or other organisms. Moreover, other metals in addition to silver are also disclosed herein and can be used in many cases interchangeably with silver. Various combinations and concentrations of the inventive compositions are also disclosed.

### DESCRIPTION OF THE PRIOR ART

It is well known that certain preparations of silver have exhibited germicidal properties. Silver was employed as a germicide and as an antibiotic before modern antibiotics were developed. In previous centuries, users would shave silver particles into their drinking water, or submerge whole silver pieces in the drinking water, for the purpose of ingesting the silver by drinking the water. It seems possible that the practice of eating with silver utensils (i.e., silverware) may have resulted from a belief in the healthful properties of silver.

There may be many reasons why administering silver suspended in solution would enhance an individual's health. It is possible that such a solution operates to inhibit the growth of bacteria, viruses, and other unwanted organisms, as well as eradicating such existing bacteria, viruses, and other organisms. It is also possible that a silver composition can have anti-inflammatory effects, sufficient to reduce, for example, swelling, burn complications and certain symptoms of asthma.

A first embodiment of the present invention describes the use of a silver composition in water to treat certain human (or, for example, certain animal) ailments. One embodiment of the invention comprises a silver composition comprising nanoparticles of silver (e.g., a majority of which are 10-50 nanometers in diameter) and which, in a preferred embodiment, may comprise an interior of metallic silver and an exterior coating or portion different from said interior (e.g., a coating of ionic silver, one or more silver oxide coating(s), (e.g., different compositions and/or different phases, etc.) which particles are suspended in water (e.g., a purified water). In a further preferred embodiment, at least 90% of such particles are 10-50 nanometers in diameter. A preferred embodiment of the invention comprises a silver composition comprising particles of silver (including certain silver oxide-coated particles of silver) wherein more than 50% of the number of particles are less than 0.015 micrometers in size and the particles are colloidally suspended (i.e., do not settle out) in water. Another preferred embodiment of the invention comprises similar particles wherein about 95% of the particles are 10-40 nanometers in diameter. In a further preferred embodiment, about 95% of the particles are 10-30 nanometers in diameter.

### SUMMARY OF THE INVENTION

The present invention is generally directed to the use of silver, at a level of 5 to 40 ppm in water (but in some cases less than 5 ppm), to kill or to disable microorganisms (including certain viruses) which are hazardous to human beings and/or animals or other living organisms. Further, the present invention is specifically directed to compositions comprising silver nanoparticles, said particles, in a preferred embodiment, comprising, for example, an interior of elemental silver and an exterior coating or partial coating or layer of, for example, one or more silver oxide(s) (e.g., ionic silver oxide, silver oxides such as Ag₂O, AgO, Ag₄O₄, etc.), said coatings of oxide being in various phase states (e.g., Ag₂O being monoclinic and/or tetragonal) and water, wherein the silver particles are placed in suspension (e.g., colloidal suspension) in the water at a level of 5-40 ppm total. One embodiment of the present invention comprises silver nanoparticles (from this point forward in the specification, it should be understood that use of the term “silver particles(s)”, or the like, when processed according to the electrochemical techniques disclosed herein, refers not only to elemental silver, but also to elemental silver particles which may have a partial or substantially complete coating of one or more compositions thereon, such coating(s) comprising one or more silver oxides on at least a potion thereof) being present in water (preferably purified water, discussed later herein), at a concentration of 5-40 ppm, wherein more than 50% of the silver particles have a maximum dimension less than 0.015 micrometers. In a preferred embodiment, most of the particles are 10-40 nm in diameter. In a more preferred embodiment, most of the particles are 10-30 nm in diameter. The composition of silver in water (as well as silver particles extracted as substantially discrete particles from silver/water mixtures made according to the invention), as well as silver/water mixtures made according to the teachings of the invention and later formed into a gel, powder, clay or zeolite (as discussed in preferred embodiments later herein) according to the teachings of this invention is/are, for example, very effective antimicrobial agent(s) and antiviral agent(s) (and in some cases anti-parasitic as well). This invention is also directed to silver compositions, of 5-40 ppm silver in water and, according to the methods of using said silver/water compositions disclosed herein, are very effective as antimicrobial agents by using said compositions as follows: (1) internally in living organisms; (2) externally on living organisms as well as externally (or internally) on a variety of surfaces, both hard and porous (e.g., countertops, food preparation surfaces, food preparation equipment, hospital surfaces, medical instruments, water lines (metal and/or plastic), air filtration devices, etc.); and (3) mixing-in silver or silver water compositions with contaminated water (e.g., waste water treatment, pond water, contaminated water containers, water lines, etc., which, preferably, have had large solids removed therefrom prior to said mixing-in) to result in a water purification process.

One preferred embodiment of the present invention is directed to compositions of silver in water made using a modification of the device and/or methods described in U.S. Pat. No. 6,214,299 (“Patent '299”), which is specifically incorporated herein by reference. Further, compositions of other metals such as, for example, copper (and copper alloys), zinc, platinum, and titanium and alloys and mixtures thereof) can be used to form other desirable metal/compositions, according to the methods of the present invention, which also have surprising efficacy.

The device and process of Patent '299 have been modified and improved to provide the silver composition of the present invention, which process is described in greater detail later herein. Essentially, the eight-silver/one common electrode device as disclosed in Patent '299 has been modified and scaled to fit a larger (e.g., 75-85 gallon) water chamber. To begin the process of manufacturing a silver/water composition in a 75-85 gallon container, approximately 70-75 gallons of relatively high purity water (e.g., filtered water, reverse osmosis water, or water that does not contain any large amounts of potential contaminants, etc.) typically containing less than 2 ppm total dissolved solids, or even more preferably, less than 1 ppm total dissolved solids, are placed in the chamber. To this is added, in a preferred embodiment, approximately five gallons of a silver/water composition produced in a prior production run. This “priming” with approximately 5 gallons is helpful, but not essential. The priming essentially provides a sufficient number of conductive silver particles to be present in the chamber so that current can flow between the various electrodes when sufficient voltage/current is achieved in a relatively short amount of time. This “priming” also results in slightly smaller initial “Taylor cones”, discussed later herein. The water chamber is equipped with an air input (typically located near a bottom portion of the water chamber) that permits a stream of air bubbles to flow through the water/silver liquid during the manufacturing thereof. It has been discovered that this approach results in an apparently improved mixing as compared to the impeller mixer described in Patent '299, as evidenced by certain increased efficiencies.

The electrode device(s) is/are operated at voltages (at least initially) on the order of, or approaching approximately ten thousand volts alternating current (with each set of silver electrodes having an individual voltage supply) as described in Patent '299. Voltages significantly higher than ten thousand volts tend to produce a solution that may have significant amounts of ionic silver dissolved therein. The present composition comprises in excess of 97% metallic silver particles present at 5-40 ppm, with essentially little to no free ionic silver present in the silver/water solution.

The silver concentration is determined according to the methods explained below. Essentially, the 75 gallon silver/water manufacturing device is operated substantially continuously and samples from the device are analyzed until the desired silver ppm concentration in the water is attained. It has been found that under the operating conditions described herein, the 10 ppm silver/water composition requires approximately one and one half days of operation; the 22 ppm silver/water composition requires approximately three days of operation, and the 32 ppm silver/water composition requires approximately six days of operation. The rate of the formation of silver particles in the silver/water compositions appears to slow as the higher concentrations of silver particles are sought. When concentrations of silver in the silver/water compositions are desired to be above 50 ppm, they take a relatively long time to achieve, within the processing parameters disclosed herein, with the highest concentration achieved to date under a reasonable amount of time being about 50 ppm. Higher silver particle concentrations are possible, if desired. However, the efficacy of the lower concentrations of silver particles against various pathogens has been so outstanding, that higher concentrations of silver particles have not been necessary to date.

The nanoparticles of silver in the silver/water compositions all have very similar overall particle size and shape characteristics, described below in the characterization section in greater detail, and unlike many conventional “colloidal silver” compositions, these silver/water compositions are completely colorless and are substantially stable with regard to moderate light and temperature changes, without the requirement for the use of any additives to assist in stability (which many prior art colloidal silvers require and/or utilize). It is believed that the components and commercial process steps utilized produce a silver/water composition that differs from other products known as “colloidal silver” in a manner which causes the silver/water compositions to have higher efficacy. Some of the salient physical property differences (e.g., particle size, composition, spectroscopy patterns, etc.) of the novel silver/water compositions of the present invention are discussed in much greater detail later herein.

The silver/water compositions of the invention are also substantially unreactive towards many materials added thereto including, for example, alone or in combination, (1) hydrogen peroxide, (2) DiSodium EDTA (disodium ethylene diamine tetra acetic acid), which actually may act as an enhancer of the silver/water compositions (e.g., may make the silver/water compositions have an even greater efficacy), (3) iodine (e.g., povidone iodine, which in some cases may show some mild reactivity), which may assist the silver/water compositions being even more pathogenic against a variety of pathogens and (4) various commercially available antibiotics (which actually may result in certain synergistic effects occurring between the silver/water compositions and the antibiotics, thus resulting in the potential for new and very desirable combination therapies being realized). Accordingly, a variety of additional materials or substances can be used in combination with (e.g., added to or supplied with) the novel silver/water compositions of the present invention to enhance, in a synergistic manner, the desirable effects that either material may exhibit alone. Specifically, in many cases (e.g., antibiotic combinations), the resulting combined effects are synergistic and exceed the individual additive effects of either material or substance alone, when combined (e.g., 2+2=6). Of course, some of the possible additives will render the novel compositions suitable only as topical or surface treatments due to their potential for internal toxicity in biological organisms (e.g., humans or animals). The amount of additive required may vary depending on many circumstances including the particular affliction (e.g., virus, bacteria, parasite, etc.) or infection, the amount of other materials present in addition to the additives, etc. However, the precise amount of additive required would be within routine experimentation to those of ordinary skill in the art. Additionally, the concentrations of the silver/water mixture can also influence the amount of additive required, also within routine experimentation for those of ordinary skill in the art.

One example of a desirable additive is hydrogen peroxide. Hydrogen peroxide is a known disinfecting agent. Hydrogen peroxide has been found to have a synergistic interaction with the inventive silver/water compositions of the invention. Hydrogen peroxide is available in concentrations of, for example, 30% by weight (% weight per volume or weight percent) or even higher. Although the higher concentrations are usable, the preferred concentrations to be used with the silver/water compositions of the present invention appear to be 30% or lower, and more preferably, fall within the range of about 1 to 5% by weight.

One preferred embodiment of the present invention is directed to compositions comprising 5 to 40 ppm silver particles, 1 to 3 weight % hydrogen peroxide, and the remainder being water (e.g., filtered or substantially purified water). Another preferred embodiment of the present invention is the use, and method of use, of compositions comprising 10 to 40 ppm silver and 1 to 3 weight % hydrogen peroxide in water as antimicrobial agents.

Another example of an additive that works favorably with the silver/water compositions of the present invention is disodium ethylene diamine tetra acetic acid also known as “Sodium EDTA” or “DiSodium EDTA” (both of which are sometimes referred to in the literature) and which may have a chemical formula as follows: (CH₂N(CH₂COOH)CH₂COONa)₂2H₂O. In another preferred embodiment of the invention, a small amount (e.g., 0.5-10 ppm, or more preferably 0.5-5 ppm, or even more preferably about 0.5 ppm) of disodium EDTA is added to, or supplied with, the silver/water compositions of the present invention. In this embodiment, it appears as though the addition of a small amount of disodium EDTA enhances the potency (e.g., enhances the bactericidal, disinfectant and/or antimicrobial properties) of the silver/water composition. Without wishing to be bound by any particular theory or explanation, it is possible that the disodium EDTA may be increasing cell wall permeability, which may enhance the overall effectiveness of the silver/water compositions of the present invention. Another preferred embodiment of the present invention is the use, and method of use, of compositions comprising 10 to 40 ppm silver and 0.5-10 ppm disodium EDTA in water as an antimicrobial agent, bactericidal agent antiviral agent and/or disinfectant.

Another example of an additive that works favorably with the silver/water compositions of the present invention is Povidone iodine. Iodine is a well known prophylaxis in medicine for treatment against a wide range of pathogens. Iodine is commercially available in various concentrations, but a commonly used, and preferred, concentration is 10%. In this preferred embodiment of the invention, a synergistic combination comprises about 25-50% by volume substitution of the silver/water mixture replacing the 10% iodine solution. While some reactions between the silver/water mixture and iodine are possible, it appears from the experimental results discussed later herein that the synergistic combination of the silver/water with povidone iodine may function as a topical disinfectant (e.g., an ointment) and/or as prophylaxis against infection in cuts, burns and or scrapes, etc. Another preferred embodiment of the present invention is the use, and method of use, of compositions comprising 10 to 40 ppm silver and povidone iodine in water as an antimicrobial agent, bactericidal agent antiviral agent and/or disinfectant.

Another preferred embodiment of the invention utilizes the silver/water compositions of the present invention in combination with various commercially available antibiotics in an approach known as combination therapy. Combination therapy has become of great interest because, in the last two decades, the spread of resistance to antibiotics has been widespread and therefore a matter of great concern globally. Infections caused by Gram-negative bacteria such as Escherichia coli, Klebsiella, Proteus, Shigella and Pseudomonas have become an increasing cause of concern as these organisms have acquired multiple drug resistance to antibiotics. A recent study to investigate the resistance pattern of gram-negative clinical isolates causing hospital infections, has shown that most of the isolates were resisitant to common antibiotics like ampicillin, gentamicin, chloramphenicol, cotrimoxazole, and the first and second generation cephalosporins. Also approximately 70% of these isolates were resistant to ciprofloxacin. In this embodiment of the invention, the silver/water mixtures (whether combined as a liquid or dried and added as a solid, thereby forming, for example, a powder, sometimes referred to herein as “Sildust”), when combined with various antibiotics, showed synergism, rather than just additive properties. Checkerboard assays showed that certain antibiotics when combined with silver/water mixtures resulted in the antibiotics being several times more effective than silver alone (e.g., silver/water mixtures combined with amikacin and cefoperazone showed an FIC index of about 0.1875, compared to the two antibiotics used in combination with each other, which resulted in an FIC index of 0.625, when both combinations were used against, for example, MRSA (Methicillin Resistant Staphylococcus aureus)) discussed in greater detail later herein. Another preferred embodiment of the present invention is the use, and method of use, of compositions comprising 10 to 40 ppm silver and various antibiotics as an antimicrobial agent and/or bactericidal agent and/or an antiviral agent in treatments referred to as “combination therapies”. The precise amount (and concentration) of silver/water mixtures according to the present invention which can be added to the conventional antibiotic therapies is a matter of routine experimentation. In particular, the specific malady being treated by a specific antibiotic course (as well as the efficacy of the antibiotic against the pathogen) will influence the amount, and concentration, of silver/water mixture required.

Although a large number of tests employing the silver/water solutions alone or in combination with various additives are presented below, it is also demonstrated herein that certain vehicles can significantly improve the results obtainable with the silver/water solutions in various situations. Specifically, it has been found that formulating the aqueous silver/water composition as a semi-solid hydrogel (sometimes referred to later herein as “Silgel” or another version referred to as “Silderm”), or even sheets of such material, significantly enhances its efficacy for certain applications. Hydrogels are typically hydrophilic gels produced by adding certain hydrophilic organic polymers to an aqueous solution—in this case a solution containing the inventive silver/water solution. However, it is anticipated that other “colloidal silver” solutions may also be formed into hydrogels, according to the teachings herein, and while such hydrogels may not be as effective as those of the present invention, those hydrogels may nevertheless have certain desirable utility. Accordingly, the present invention is intended to cover certain aspects of those hydrogels as well. As would be expected, the hydrogel improves the retention of the silver on a surface area, such as a wound on a skin surface area. For wound care, a hydrogel or sheet material also has the significant advantage of protecting the tissues surrounding the wound and preventing desiccation, which factors often enhance wound healing. Most significantly, the hydrogel does not appear to interfere, substantially, if at all, with the antimicrobial properties of the silver nanoparticles of the present invention. Further, these hydrogels function as excellent hand or skin cleansers, as well as skin protectants (e.g., placing the hydrogels on hand(s) so that should the hand(s) come into contact with pathogens, the skin protectant gel could assist in preventing infections due to, for example, cuts or abrasions, thereby functioning as a prophylactic), thus making the gels of great utility to the healthcare or wellness field.

In particular, clean hands are thought to be the single most important factor in preventing the spread of dangerous germs and antibiotic resistance in health care settings. Most hygienic hand washes used in modern day medicines are alcohol-based and have several limitations. Primary among these limitations is damage to skin that is caused by repeated exposure to alcohol-based products. In some cases (1) irritant contact dermatitis as well as (2) allergic contact dermatitis has also been reported. This reduces compliance of many health care workers in the use of hand hygiene products.

Another factor causing non-compliance of effective hand hygiene practices is the fact, that being liquids, hand hygiene products are, typically, permanently fixed above washbasins or sinks. This results in the health care personnel having to move from the patient's bedside to the washbasin and back to the next patient. If a hand wash was available as a “rub on” this problem could be obviated, thus ensuring better compliance. The hydrogel products of the present invention have been shown to reduce bacterial counts of indicator organisms by significant amounts, over extended periods of time, as discussed in greater detail herein, thus resulting in a viable alternative hand hygiene product. Accordingly, the hydrogel products of the present invention have also shown great utility as “skin protectants”, protecting normally healthy skin from various pathogenic materials in a prophylactic manner.

In another preferred embodiment of the invention, a silver-based product can be at least partially, or in some cases substantially completely, substituted for the silver/water compositions of the present invention. Specifically, it has been discovered that Silver EDTA (or AgEDTA), by itself, has very intriguing antimicrobial characteristics. In particular, as discussed above, DiSodium EDTA is a useful additive to the silver/water compositions of the present invention. However, EDTA (edetic acid) is an excellent synthetic chelating agent. EDTA (C10-H16-N2-O8) is permitted for use in human foods and is often added to soft drinks as a preservative. EDTA has also been used in heavy-metal chelating therapy for humans. However, what has not been considered is the use of AgEDTA as an antimicrobial (e.g., by itself or in combination with other therapies, such as those disclosed herein). Mass market applications such as the meat or protein production and processing industry, soap industry, detergent industry (e.g., personal and household care products), agricultural or farming of crops industry and heath care industry may be well suited for a powder form of stable silver which may provide many powerful health or wellness benefits (e.g., both therapeutic and prophylactic). In particular, AgEDTA is readily available and is relatively simple to manufacture, store and transport. This embodiment of the invention recognizes a new use for AgEDTA, namely, using the powder AgEDTA for the health or wellness of humans, plants and/or animals and/or the treatment of certain disorders in animals and humans (e.g., can be used as a therapeutic treatment and/or as a prophylactic). Akzo-Nobel currently manufactures an acceptable AgEDTA. Other silver chelating or complexing agents such as, for instance, silver EDDS, silver curcuminate, silver berberine, and silver tetracycline also exhibit antimicrobial properties and the use of these materials for the health and wellness of humans or animals is also new and unrecognized in the prior art. Various other organic structures can be utilized to carry and/or deliver silver and/or silver ions to various efficacious locations in or on biological structures. Once again, the amount of AgEDTA required will vary depending on the particular biological issues surrounding the need (e.g., treatment requirements and/or prophylaxis).

In another preferred embodiment of the invention, additional silver-based inorganic products can be at least partially, or in some cases, substantially, completely substituted for the silver/water compositions of the present invention.

Specifically, silver (e.g., silver ions, silver metal, Ag+) can be controllably attached or fixed, for example, on or between clay layers and/or within cages in zeolites. Such fixing can occur by controlling the charge of, for example, the silicate layer, the charge of the zeolite cage, as well as the distances between layers or the size of the zeolite cage. In this regard, silver can be attached or bonded tightly or relatively loosely, depending on the particular health or wellness application and the point of interaction between the silver and the biological (e.g., on the surface of the biological, or in an internal portion, or combination of internal portions, etc.). Accordingly, resultant products may include products that are quite fluid and are thus drinkable or sprayable; as well as products that are gel-like or paste-like and are spreadable on surfaces like gels or pastes.

Any of the metals discussed herein can be held within a crystalline or amorphous clathrate of one or more atomic layers of oxygen or oxygen-containing molecules. Certain metal/clathrate structures have been shown to have unexpected efficacy. Further, in addition to silver being incorporated into a structure of an oxide layer (e.g. clays) and networks (e.g. zeolites) silicates, phosphates, and oxides such as hydrotalcytes can also be utilized. Still further, desirable clays or mica families that are capable of being utilized with the present invention (and which are capable of having different surface charges and/or different distances between layers) include, for example, illites, montmorillonites, chlorites, and vermiculites.

Clays or micas, as well as zeolites, are very desirable as metal(s) ion carriers for several reasons including many are naturally occurring or easily derived, the particles can be maintained in desirable colloidal size range which render them, for example, suspendable in liquids (e.g., water) and are typically very biologically friendly (e.g., little or no side-effects). In this regard, once silver is placed within, for example, a clay or zeolite clathrate, the molecules are then heated to moderate temperatures (e.g., 100-200° C.) to fix the silver to or within the clathrate. All of these materials can be made in a wide range of viscosities from being very fluid to being very viscous.

In general, the electronic levels in elements such as cations, in any given valence state, can be changed when that element cation is coordinated by various anions. In particular, the more covalent the bond, the more the energy levels can be changed. It is very likely that small to moderate changes in the electronic structure of silver will occur when silver is surrounded (or coordinated) by differing numbers of oxide ions. This change in electronic structure for cations, such as silver cations, should occur in any of the various silver oxide structures. Still further, there is a more general manner in which silver can be placed into an oxygen clathrate or cage. In this regard, by exchanging, for example, the sodium cation in a structure, with a silver cation, then the sodium ions sitting in the exchangeable cavities or spaces (e.g., either on or between sheets of clay or within networks of zeolites) can occur. In general, the ability of one material to exchange cations is known as its “CEC” or “cation exchange capacity”. The units for CEC are typically referred to as “meq/100 grams” or milliequivalents per one hundred grams. In general, the higher the CEC number, the greater the ability a material has to accept cations (e.g., silver cations). Accordingly, many oxygen-coordinated silver compounds can serve the role of a carrier of silver (or other metals) and are thus capable of acting as therapeutic agents by themselves, or in combination with other therapeutic agents.

Still further, silver-metal or silver-ions incorporated into a silica gel by diffusing and drying are also desirable mechanisms for delivering metal(s) of the present invention.

In another preferred embodiment of the invention combinations of the aforementioned particles, organic and/or inorganic structures can be utilized to positively affect the health and wellness of humans and animals. Specifically, metal particles, according to the invention, can be used alone, as discussed above. Further, the metal particles can be combined with, for example, the organic compounds discussed above (e.g., AgEDTA). Still further, the metal ions according to the present invention can be combined with any of the inorganic compounds (e.g., clays or zeolites). Still further, metal ions of the present invention can be combined with both the organic molecules (e.g., AgEDTA) and the inorganic molecules (e.g., clays or zeolites). This combination of silver metals or silver ion delivery systems can be constructed so that, for example, an internal consumption of any of the aforementioned silver delivery systems can result in silver being delivered to different portions of, for example, an organism. In particular, for example, in humans, certain silver could be absorbed by the mouth, through the gut, as well as through the large and/or small intestine, etc. Further, depending on, for example, the amount of clay(s) or zeolite(s) relative to water (as well as various gelling compounds disclosed herein) the resultant product(s) of the present invention can be very liquidy (low viscosity) to very viscous (high viscosity). In this regard, in general, the more clay or zeolite provided relative to water (as well as gelling agent) the more viscous the final product.

### DETAILED DESCRIPTION OF THE INVENTION

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide an improved silver/water composition (sometimes referred to herein as silver nanoparticles dispersed in water) which, may be used by itself or in combination with (e.g., mixed with or supplied substantially contiguously therewith) other disclosed materials, and which may be formed into various hydrogel or paste compositions, all of which exhibit significant abilities to kill human and/or animal pathogens both in vivo and in vitro.

Generally, the present invention represents a novel approach to killing or disabling microorganisms which are hazardous to human beings and/or animals by the use of silver nanoparticles in water, at a concentration of 5 to 40 ppm silver; or active silver particles contained in, for example, AgEDTA, and/or other compounds discussed herein. Depending upon the application, and/or the additives present, the silver/water composition may be used internally or externally. Depending on the application, the silver/water composition may also contain various desirable additives many of which have not been specifically listed herein, but will become apparent as having utility to those of ordinary skill in this art.

### BRIEF DESCRIPTION OF THE FIGURES

FIGS. 1-6 show TEM photomicrographs taken, at various magnifications, of silver particles formed in silver/water compositions formed according to the present invention.

FIG. 7a-7d show TEM photomicrographs generated from a different TEM and utilizing a different technique from that used to generate FIGS. 1-6; and FIG. 7e shows an EDS (EDAX) spectrum of silver particles taken from the silver/water composition of the present invention.

FIG. 8 shows an electron diffraction pattern taken from a silver particle from the silver/water composition of the present invention.

FIG. 9 includes three SEM photomicrographs which together show possible electron beam damage to silver particles taken from silver/water compositions of the present invention.

FIG. 10 shows an SEM photomicrograph of a new silver electrode prior to being used in the process according to the present invention.

FIGS. 11, 12 and 13 show EDS elemental analyses of the portions 1, 2 and 3, respectively, shown in FIG. 10.

FIG. 14 shows an SEM photomicrograph of the tip of an electrode used to manufacture silver/water compositions according to the present invention.

FIGS. 15 and 16 show EDS elemental analyses of the portions 1 and 2 respectively, shown in FIG. 14.

FIG. 17 shows a SEM photomicrograph taken at approximately 3500× of the used silver electrode tip.

FIGS. 18a and 18b are TEM photomicrographs of silver particles taken from GNC Liquid Silver Dietary Supplement (25 ppm).

FIGS. 19a and 19b are TEM photomicrographs of silver particles taken from a colloidal silver product known as “Silverado”.

FIGS. 20a and 20b are TEM photomicrographs of silver particles taken from a colloidal silver product known as Vitamin World Bioorganic Advanced Colloidal Minerals (3 ppm).

FIG. 21 is an overlay comparison of five TEM photomicrographs of silver particles, two of which are from silver particles from the present invention and three of which are from silver particles taken from commercially available colloidal silvers.

FIGS. 22a and 22b show seven different Raman spectra, three of which correspond to silver/water compositions of the present invention, one corresponds to pure water, one corresponds to deionized water and two correspond to commercially available colloidal silver products.

FIG. 23a shows two Raman spectra corresponding to inventive silver/water compositions; and FIG. 23b shows three Raman spectra which correspond to three commercially available colloidal silver products.

FIG. 23c shows another Raman spectrum corresponding to the inventive silver/water compositions.

FIG. 24a shows a Raman spectrum of a silver/water composition of the present invention; and FIG. 24b shows three Raman spectra of silver/water, zinc/water, and copper/water compositions.

FIG. 25 shows a diagram of potential interactions in a disc diffusion test for bacterial synergy.

FIG. 26 shows checkerboard titrations and graphs depicting additive, synergistic and antagonistic effects in combination therapy.

FIG. 27 shows photographs of the sensitivity of MDR isolates to 10 ppm silver/water mixtures.

FIG. 28 shows photographs of antibiotic combinations for MRSA.

FIG. 29 shows photographs of antibiotic combinations for E. coli.

FIG. 30 shows photographs of antibiotic combinations for Pseudomonas.

FIG. 31 shows a graph of “instantaneous” applied voltage, and instantaneous silver concentration as a function of process time during the silver/water composition formation process.

FIG. 32 shows a graph of instantaneous silver concentration as a function of process time using atomic absorption spectroscopy and electrical conductivity measurement techniques, respectively. This Figure also shows silver concentration after 32 hours of production, and after homogenization.

FIG. 33 shows a graph of instantaneous applied voltage, power factor and silver concentration as a function of process time during the inventive silver/water composition formation process.

FIG. 34 is a graph showing moisture loss of SILDERM.

FIG. 35 is a graph showing moisture uptake of SILDERM.

FIG. 36 is a photograph showing antibacterial activity of silver chelates (Ag EDTA manufactured by Akzo-Nobel) against pseudomonas aeruginosa (MDR).

FIG. 37 is a photograph showing antibacterial activity of silver chelates (Ag EDTA manufactured by Alpha Chemicals) against pseudomonas aerugimosa (MDR).

FIG. 38 is a photograph showing the sensitivity of SILDUST against E. coli (MDR).

FIG. 39 is a graph showing the antiviral activity of SILDUST as a function of exposure time.

FIG. 40 is a photograph of a central test plate, showing growth of plaques.

FIG. 41 is a photograph of the test plate, showing no plaques after three hours, and thus showing antibacteriophage activity of SILDUST.

FIG. 42 shows four X-ray diffraction patterns of a 200 ppm inventive silver/water composition; and four reference X-ray diffraction files superimposed thereon (i.e., AgO, Ag₂CO₃, Ag and Ag₂O).

FIG. 43 shows a “TGA” analysis of Ag₄O₄, as well as “DTA” analysis of Ag₄O₄.

FIGS. 44a and 44b are SEM microphotographs that correspond to inventive kaolinite/silver mixtures made according to the present invention.

FIGS. 45a and 45b are EDS (EDAX) analyses corresponding to photomicrographs of 44a and 44b, respectively.

FIG. 46 is an SEM photomicrograph of a novel zeolite/silver mixture made according to the present invention.

FIG. 47 is an EDS (EDAX) analysis of a zeolite Linde 4A containing silver substituted therein, and made according to the present invention.

FIG. 48a shows a UV-Vis spectra of a 10 ppm silver/water solution and a 32 ppm silver/water solution over a 190 nm-400 nm wavelength range (both made according to the present invention); and FIG. 48b shows a UV-Vis spectra of the same samples over a 190 nm-250 nm range.

### PREFERRED EMBODIMENTS

Non-limiting preferred embodiments are presented in the following:

A composition comprising silver nanoparticles, colloidally suspended in water, wherein the total content of silver is between 5 and 40 ppm, which composition kills or disables microorganisms which are hazardous to humans and/or animals.

A composition comprising silver nanoparticles, colloidally suspended in water, wherein the total content of silver is about 10±2 ppm, which composition kills or disables microorganisms which are hazardous to humans and/or animals.

A composition comprising silver nanoparticles, colloidally suspended in water, wherein the total content of silver is about 22±2 ppm, which composition kills or disables microorganisms which are hazardous to humans and/or animals.

A composition comprising silver nanoparticles, colloidally suspended in water, wherein the total content of silver is about 32±3 ppm, which composition kills or disables microorganisms which are hazardous to humans and/or animals.

A hydrogel composition made from a precursor silver/water composition comprising silver nanoparticles, colloidally suspended in water, wherein the total content of silver in the precursor material is, preferably about 32±3 ppm (but could be more or less), which hydrogel composition kills or disables microorganisms which are hazardous to the human body and functions as, for example, a skin cleanser, wound healer and/or skin protectant or skin disinfectant.

It should be appreciated that specifying the total amount of silver nanoparticles in a silver/water composition does not completely specify the material. As the nanoparticles comprising the composition are made smaller, a given concentration of silver will represent a larger number of particles. In addition, the total surface area for a given silver concentration will increase. Therefore, particle sizes and ranges of particle sizes are important parameters for defining an effective inventive silver/water composition. Further, coating(s) such as oxide coatings (e.g., partial or substantially complete) on said silver particles may also effect the efficacy of the silver/water compositions of the invention, such coatings inherently resulting from the processing conditions of the invention. However, similar coatings on silver particles achieved by other processes (as well as metals other than silver such as zinc, copper, copper alloys, titanium, platinum, and alloys or mixtures thereof) are also contemplated as being within the metes and bounds of this invention. Accordingly, whenever silver is referred to herein the use of various others of the alternative metals discussed herein should also be considered as exhibiting possible efficacy, depending on the particular biological conditions (e.g., specific pathogens involved).

A further class of embodiments is any of the above-described compositions, wherein more than 50% of the silver nanoparticles have a maximum dimension less than 0.015 micrometers.

A further class of embodiments is any of the above-described compositions, wherein more than 75% of the silver nanoparticles have a maximum dimension less than 0.015 micrometers.

A further class of embodiments is any of the above-described compositions, wherein more than 90% of the silver nanoparticles have a maximum dimension less than 0.02 micrometers.

A further class of embodiments is any of the above-described compositions, wherein more than 75% of the silver nanoparticles have a minimum dimension greater than 0.005 micrometers.

A further class of embodiments is any of the above-described compositions, wherein more than 90% of the silver nanoparticles have a minimum dimension greater than 0.005 micrometers and less than 0.040 micrometers.

A further class of embodiments is any of the above-described compositions, wherein the silver nanoparticles comprise both silver in the zero-valent, that is, metallic, oxidation state (Ag(0)) in a core or central portion thereof, and at least one coating of silver in an ionic oxidation selected from the group consisting of Ag(I), Ag(II), and Ag(III), with a coating of AgO, Ag₂O, and/or Ag₄O₄ being most likely present on at least a portion of (or substantially all of) the metallic silver core.

A further class of embodiments is any of the above-described compositions, wherein the silver particles comprise both silver in the zero-valent, that is metallic, oxidation state (Ag(0)) and a coating of silver oxide with the stoichiometry AgO or Ag₂O or another known stoichiometry, which is stable under the process conditions used to make the Ag₂O novel silver/water compositions of the invention.

Further experimental evidence shows that silver oxide coatings inherently occurring on at least a portion of the particles of the present invention is at least partially in the form of, for example, Ag₄O₄—that is, silver II oxide. In a molecule of this material two of the silver atoms may be in the 1⁺ state (silver I) while the other two silver molecules may be in the 3⁺ state (silver III). Further, under certain conditions silver can be present in the 2⁺ (silver II) state, resulting in at least partial coatings of, for example, Ag₂O. These coatings inherently result from the processing conditions of the invention (e.g., those conditions created at and around the electrode/water interface) and may be very important in the overall efficacy of the silver/water compositions of the invention. The exact composition of the coatings has been difficult to determine to date, but experimental detail has been provided in the characterization section later herein.

A further class of embodiments is the combination of any of the above-described silver/water embodiments with hydrogen peroxide, at a level of 1-3 weight % hydrogen peroxide in the final product.

A further class of embodiments is the combination of any of the above-described silver/water embodiments with DiSodium EDTA, at a level of 0.5-10 ppm in the final product.

A further class of embodiments is the combination of any of the above-described silver/water embodiments with about 50-75% by volume substitution of 10% povidone iodine replacing about 25-50% of the silver/water mixture in the final product.

A further class of embodiments is the combination of any of the above-described silver/water embodiments with various commercially available antibiotics (whether in liquid form or powder form) to result in synergistically effective combination therapies.

A further class of embodiments is the methods for using all of the above-mentioned compositions against human or animal pathogens, either: (1) internally, (2) externally or (3) both internally and externally.

A further class of embodiments includes the use of AgEDTA for human and/or animal health or wellness.

A further class of embodiments includes the use of other silver agents such as, for example, silver EDDS, silver curcuminate, silver berberim, and silver tetracycline.

A further class of embodiments includes the use of other metals such as zinc, copper, copper alloys, titanium, platinum and alloys or mixtures thereof, interchangeably with silver in both the preparation and the processing application methods disclosed herein. For the sake of brevity, silver is referred to predominantly herein, however, it should be understood that the other metals disclosed herein may be equally beneficial.

In another preferred embodiment of the invention, additional silver-based inorganic products can be at least partially, or in some cases, substantially, completely substituted for the silver/water compositions of the present invention. Specifically, silver (e.g., silver ions, Ag+, silver metal) can be controllably attached or fixed, for example, between clay layers and/or within cages in zeolites. Such fixing can occur by controlling the charge of, for example, the silicate layer, the charge of the zeolite cage, as well as the distances between layers or the size of the zeolite cage. In this regard, silver can be attached or bonded tightly or relatively loosely, depending on the particular health or wellness application and the point of interaction between the silver and the biological (e.g., on the surface of the biological, or in an internal portion, or combination of internal portions, etc.). Accordingly, resultant products may include products that are quite fluid and are thus drinkable or sprayable; as well as products that are gel-like or paste-like and are spreadable on surfaces like gels or pastes. Any of the metals discussed herein can be held within a crystalline or amorphous clathrate of one or more atomic layers of oxygen or oxygen-containing molecules. Certain metal/clathrate structures have been shown to have unexpected efficacy. Further, in addition to silver being incorporated into or onto a structure of an oxide layer (e.g. clays) and networks (e.g. zeolites) silicates, phosphates, and oxides such as hydrotalcytes can also be utilized. Still further, desirable clays or mica families that are capable of being utilized with the present invention (and which are capable of having different surface charges and/or different distances between layers) include, for example, illites, montmorillonites, chlorites, and vermiculites.

Clays or micas, as well as zeolites, are very desirable as metal(s) ion carriers for several reasons including many are naturally occurring or easily derived, the particles can be maintained in desirable colloidal size range which render them, for example, suspendable in liquids (e.g., water) and are typically very biologically friendly (e.g., little or no side-effects). In this regard, once silver is placed, for example, within or on a clay or zeolite, the molecules are then heated to moderate temperatures (e.g., 100-200° C.) to fix the silver to or within the clathrate. All of these materials can be made in a wide range of viscosities from being very fluid to being very viscous.

Still further, silver-metal or silver-ions incorporated into a silica gel by diffusing and drying are also desirable mechanisms for delivering metal ions of the present invention.

In another preferred embodiment of the invention combinations of the aforementioned particles, organic and/or inorganic structures can be utilized to positively affect the health and wellness of humans and animals. Specifically, metal particles, according to the invention, can be used alone, as discussed above. Further, the metal particles can be combined with, for example, the organic compounds discussed above (e.g., AgEDTA). Still further, the metal ions according to the present invention can be combined with any of the inorganic compounds (e.g., clays or zeolites). Still further, metal ions of the present invention can be combined with both the organic molecules (e.g., AgEDTA) and the inorganic molecules (e.g., clays or zeolites). This combination of silver metals or silver ion delivery systems can be constructed so that, for example, an internal consumption of any of the aforementioned silver delivery systems can result in silver being delivered to different portions of, for example, an organism. in particular, for example, in humans, certain silver could be absorbed by the mouth, through the gut, as well as through the large and/or small intestine, etc. Further, depending on, for example, the amount of clay(s) or zeolite(s) relative to the water (as well as various gelling compounds disclosed herein) the resultant product(s) can be very liquidy (low viscosity) to very viscous (high viscosity). In this regard, in general, the more clay or zeolite provided relative to the water (as well as gelling agent) the more viscous the final product.

### EXAMPLES

Formation of Composition

Compositions of silver/water can be made according to procedures set forth in U.S. Pat. No. 6,214,299, the subject matter of which is specifically incorporated by reference herein.

A preferred method for producing a composition comprising silver according to this invention utilizes a electrochemical cell comprising electrodes and comprises the steps of:

(a) placing at least two silver electrodes in contact with a quantity of high purity water;

(b) conveying electrical current through the silver electrodes to thereby separate particles of silver from said silver electrode in a manner sufficient to cause production of suspended silver particles within the water; and

(c) agitating the water during said production of suspended silver particles to thereby disperse the silver particles into a more uniform concentration within said water such that a high quantity and substantially uniform distribution of suspended silver particles can be produced per batch.

Another preferred method for producing a composition comprising silver/water compositions utilizes an electrochemical cell and comprises the steps of:

(a) establishing an electrical circuit comprising a current source, and a first conductor electrically connected to said current source and a second conductor electrically connected to said current source, wherein said first conductor is disposed spaced apart from said second conductor, and wherein at least one of the conductors is made of elemental silver, or alternatively, zinc, copper, copper alloys, titanium, platinum and alloys or mixtures thereof;

(b) closing the circuit by placing the first conductor and the second conductor in communication with a fluidic resistor;

(c) operating the current source to supply alternating current simultaneously to the first conductor and the second conductor such that voltage is increasing and decreasing within the first and second conductors in alternating tandem to thereby cause silver (or other metal) particles to separate from the first electrode and enter the fluidic resistor and become disposed in suspension within said fluidic resistor; and

(d) selectively adjusting the electrodes by moving them toward the fluidic resistor to compensate for decrease in electrode length due to gradual separation of silver particles therefrom to thereby prevent arcing from occurring between the electrodes and said fluidic resistor and to maintain a desirable current density at the tip of the electrodes.

Each water chamber or tank which produces silver/water compositions has a power supply consisting of eight transformers (an acceptable transformer for use in the present invention is Franceformer, Part No. 48765) rated for 120 VAC input and for 10,500 VAC maximum output at 30 milliamps. Each transformer was preferably equipped with a 45-microfarad capacitor (such as Aerovox, Part No. M24P3745MP2) wired in parallel across the transformer input lead.

The combination of the transformer and capacitor may be beneficial in some cases and very desirable in others. In particular, the transformer assists in bringing the voltage and current sine waves of AC power into phase with each other. The degree to which the voltage and currents are in phase with each other is known as the power factor. The closer the power factor is to 1.0, the closer the phases match between volts and amps and the more power is delivered to the electrodes (e.g., power is typically determined by multiplying volts times amps).

Each tank is fitted with a transparent cover made of, for example, a suitable polymer, and is constructed to receive eight electrode sets. Each electrode set is comprised of a fixed electrode, made from, for example, 18 gauge silver plate, flanked by two consumable electrodes, made from, for example, 18 gauge silver wire (0.9999 purity). The electrodes are preferably bent in half in the middle and the ends twisted together in a double helix to obtain a desirable voltage and power density combination. Each electrode set is powered by one transformer.

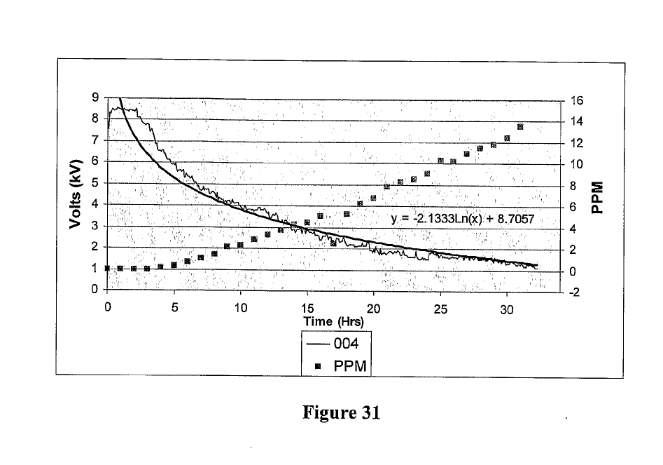

As each tank is filled for production, the electrodes are adjusted so that the fixed electrodes are in good contact with the water (e.g. , at least ⅓ to ½ of the plates are submerged), and the consumable electrodes are above the water surface. When the power supply is energized, the water rises and forms a cone-like structure around each consumable electrode. This cone-like structure is known in the literature as a “Taylor cone”. Initially, the water is very pure, and thus possesses high electrical resistance. Accordingly, when utilizing, for example, a fixed current, 10,000 volt transformer, the applied voltage across the electrodes can be initially very high, for example, about 6500-8500 volts, and the consumable electrodes can be 5-10 mm above the water surface, thereby achieving a desirable voltage and a desirable current density at the consumable electrodes. This results in a relatively large Taylor cone due to the low conductivity of the water relative to the high conductivity of the electrode (e.g., a large field is created). The silver nano-particle product is formed as silver particles are removed from the consumable electrodes at the air-water-silver electrode interface. As the water takes on more and more silver particles, the electrical resistance of the water/silver mixture drops. In a fixed current or current limited arrangement, the applied voltage will then drop or decrease as a function of time (see, for example, FIG. 31). Accordingly, the consumable electrodes are, typically, lowered to be closer to the surface of the water, for example, perhaps only 1-2 mm above the surface. In simple terms, the Taylor cones will then be much smaller because of a lesser difference in conductivity between the electrodes and the water (e.g., a lesser field is present). In general, the consumable electrodes and/or water level should be adjusted appropriately during the production process to maintain the initial geometry. Even though the Taylor cones become smaller during this process (thus representing, for example, metal particles going into the solution) small Taylor cones will still be present at the end of the processing. The water in each tank is air agitated during the entire process to maintain homogeneity.

Once a desirable or target ppm of silver in the silver/water solution is attained, the product can then be pumped, if desired or needed, through a 1 micron filter into one of several very large, for example, 2,300-6,500 gallon capacity holding tanks, and analyzed before being bottled for shipment. Analysis is performed by a digestion process using heat and nitric acid, and analysis occurs using a Perkin-Elmer Analyst 300 Atomic Absorption Spectrophotometer. The produced silver/water composition can thereafter be combined with other ingredients to make a hydrogel, a sheet material, or can be bottled as is, or can be combined (e.g., either as a liquid or dried and added as a powder) with other additives, as discussed elsewhere herein.

With reference again to FIG. 31, what is shown is the real time voltage drop and silver concentration data as a function of time corresponding to a run for forming the inventive silver/water compositions. Clearly, as the run progresses, the voltage decreases as the silver concentration increases. A corresponding decrease in the size of the Taylor cones on each consumable electrode is also noticed. The silver concentration data on this graph should not be taken as quantitative, however, but representative, due to sampling and mixing issues (e.g., the silver/water mixture may not be completely homogenous at any one sampling moment).

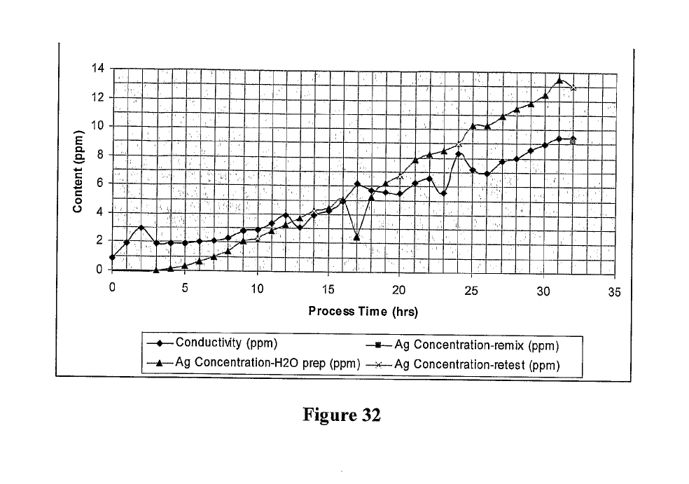

Referring now to FIG. 32, what is shown are two plots of silver concentration for this same run, as well as several additional concentration data points. The gray line with the squares denotes the instantaneous silver concentration (as determined by atomic absorption spectroscopy) based on a 60 ml sample obtained by pipette from the approximate mid-depth of the tank and about halfway between the center and the wall of the tank. The black line with the diamonds denotes the instantaneous silver concentration as roughly approximated by a previously calibrated device measuring the electrical resistivity of the above-mentioned 60 ml aliquot of liquid. In terms of raw resistivity data, the water initially (i.e., time=zero) had an electrical resistivity of about 175 kilo-ohm centimeters. In contrast, at the 31-hour mark into the run, the water/silver mixture had a resistivity of about 62.7 kilo-ohm centimeters.

Immediately below the concentration/resistivty data point at the 32-hour mark is a single data point present as a “square”. This data point denotes the silver concentration as determined by atomic absorption spectroscopy after turning off the high voltage, but letting the bubbler/mixer continue operating for another 20 hours to homogenize the mixture.

One conclusion that can be made from FIG. 32 is that the silver initially may not be homogeneously distributed throughout the tank as the silver is formed, despite the presence of the bubbler/mixer operating during the course of the run. Rather, there may be a certain lag time after completion of silver additions to the bath before the bubbler/mixer can “catch up”, and homogeneously distribute the silver throughout the water.

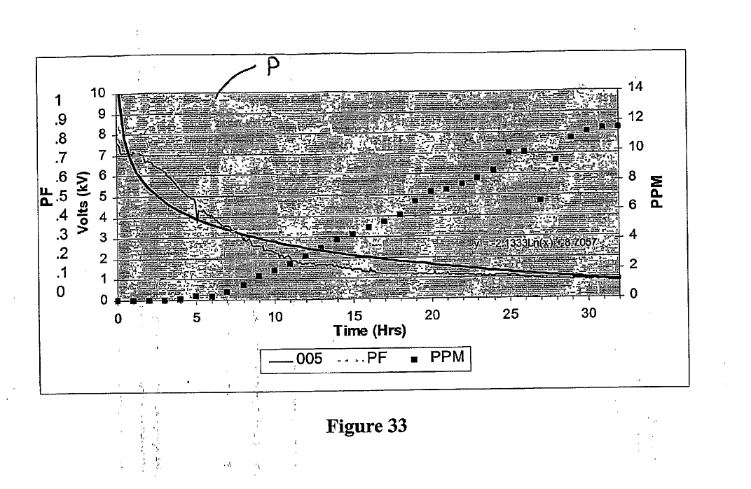

FIG. 33 is another graph of instantaneous voltage and silver concentration as a function of time during the course of a silver/water production run. This graph furthermore shows the instantaneous “power factor” of the power supply transformer. Thus, the power factor started out at about 0.8, increased to a maximum of about 0.97 around 6 hours, and decreased to a low of about 0.6 after about 30 hours. Further, the voltage/time data was mathematically fit to the equation y=−2.1333 Ln(x)+8.7057, where y corresponds to voltage and x corresponds to time. The ppm of silver in water is represented by the “squares” and begins around 1 ppm and reaches a maximum of about (e.g., due to the water not being completely pure after filtering) 11 ppm after about 30 hours.

Physical Characterization

The analysis of the silver content in the silver compositions of this invention may be performed by (acetylene) flame-atomic absorption spectroscopy (FAAS), inductively coupled plasma (ICP), atomic emission spectroscopy (AES) or other techniques known to one of ordinary skill in the art to be sensitive to silver in the appropriate concentration range, If the particles of the silver composition are small and uniformly sized (for example, 0.01 micrometers or less), a reasonably accurate assay may be obtained by running the colloid directly by atomic absorption or ICP/AES. This is because the sample preparation for atomic absorption spectroscopy ionizes essentially all of the silver allowing its ready detection.

If the compositions comprise particles as large as 0.2 micrometers, it is preferred to use a digestion procedure. The digestion procedure is not necessarily ideal for silver compositions that may have been manufactured or stored in contact with halides or other anionic species that may react with finely divided silver, or combined with protein or other gelatinous material. An embodiment of the digestion procedure is as follows:

1. Take a 10 ml aliquot of a thoroughly mixed or shaken silver composition to be analyzed, and place it in a clean polycarbonate bottle or other container of suitable material (generally, the bottle) with a tight fitting lid. A size of 30-100 ml is preferred.

2. With a micropipette or dropper, add 0.1 ml of nitric acid, reagent grade to the silver composition in the bottle.

3. With the lid of the bottle tightly in place, heat the silver composition to at least about 80° C., and preferably about 90° C.-100° C. with mild agitation for a time sufficient to dissolve the silver—dissolution is essentially instantaneous.

4. Allow the resulting mixture to cool to room temperature with the lid in place. Shake the bottle thoroughly. This digestion procedure also dissolves any silver oxide surface layer that may be present on the silver particles.

5. Utilize atomic absorption spectroscopy, ICP/AES, or equivalent means to analyze the silver content of the silver mixture. Preferably, one will utilize a freshly prepared standard or standards, preferably prepared according the equipment manufacturer's instructions, with appropriate dilution as needed.

6. When reporting results, one must take into account all dilutions during preparation, including the 1% dilution caused by addition of the nitric acid.

The silver concentration of the silver/water compositions of the present invention corresponding to the data in FIGS. 31, 32, 33, etc., was determined using a Perkin Elmer AAnalyst 300 atomic absorption (AA) spectrometer. Samples of the inventive silver/water compositions were digested according to the procedure described above.

Principle

The Perkin Elmer AAnalyst 300 system consists of a high efficiency burner system with a Universal GemTip nebulizer and an atomic absorption spectrometer. The burner system provides the thermal energy necessary to dissociate the chemical compounds, providing free analyte atoms so that atomic absorption occurs. The spectrometer measures the amount of light absorbed at a specific wavelength using a hollow cathode lamp as the primary light source, a monochromator and a detector. A deuterium arc lamp corrects for background absorbance caused by non-atomic species in the atom cloud.

Analysis of Physical/Chemical Form of Silver and Silver/Water Compositions

A. Introduction

A sample of a composition, nominally containing 22 ppm silver in water, was analyzed by time-of-flight secondary ion mass spectrometry (TOF-SIMS) in order to determine the form of silver in the composition. The conclusion is that the bulk of the silver exists as silver(0) (that is, metallic silver) and that there is a surface coating which as on average a composition of, for example, silver(II)oxide (AgO). As mentioned above silver(II)oxide is usually a stoichiometric combination of silver(I) and silver(III).

B. Experimental Procedure

A few drops of the 22 ppm inventive silver composition were evaporated to dryness on a silicon substrate at ambient temperature. The residue was analyzed by TOF-SIMS, and is denoted as the sample. A reference silver (II) oxide (AgO) material was analyzed by placing a few particles of the reference powder as received from the vendor on a silicon substrate, and is denoted as the reference.

The Time-of-Flight Secondary Ion Mass Spectrometry technique (TOF-SIMS) is based on the principle of bombarding a solid sample with a pulsed, finely focused beam of primary ions, and then analyzing the secondary ions produced from the surface of the sample via a time-of-flight mass spectrograph. This analytical technique is surface sensitive, deriving its information from a layer that extends to approximately 20 to 40 Å (one Angstrom=1×10−4 micrometers) below the surface. The TOF-SIMS technique is normally used as a survey tool to identify the composition of unknown samples. It is capable of quantification if the appropriate microanalytical standards are available for calibration. This analysis was carried out using standard high mass-resolution conditions.

C. Results

Negative ion mass were obtained for the Ag(II)O reference material and the product sample, respectively. The mass spectral region for both spectra showed the presence of more than one species of silver oxide which was most likely present as at least a partial coating on the silver particles. The data suggest that silver(II) is the average oxidation state of the silver present on the surface of the sample particles. The silver oxide (e.g.,AgO) signals exhibit significantly higher intensity in the reference sample compared to the product sample which is probably because metallic silver is dominant in the sample. It will be appreciated that as the average particle size in the sample is decreased the ratio of silver to silver oxide will also decrease as more silver oxide will be present.

Size/Morphology/Composition Analysis

It is likely that the unusual effectiveness of the silver/water preparations described herein is due to the relationship between the surface properties/inner properties (e.g., oxide/metal) of the particles and/or the size distribution of the silver nanoparticles and/or the morphology of the silver nanoparticles. The smaller the average particle size, the greater the surface area and the greater the contribution of the particular surface chemistry. However, if the particles are excessively small there can be a loss of stability and/or other interactions that may negatively affect the product. The silver/water compositions of the instant invention are remarkable because they are stable in essentially pure water without surfactants, etc. (e.g., many prior art “colloidal” silvers require proteins to maintain the silver particles in suspension). Also, the silver/water compositions are essentially colorless while other colloidal silver preparations (particularly with larger particle sizes) usually show colors. These properties are a result of the manufacturing conditions, as discussed above herein.

Digital analysis of the composition showed that there is an average particle diameter of 0.0106 micrometers with a range of 0.005 micrometer to 0.0851 micrometers. However, size distribution analysis shows that more than 95% of the particles were between about 0.005 micrometers and about 0.015 micrometers in diameter.

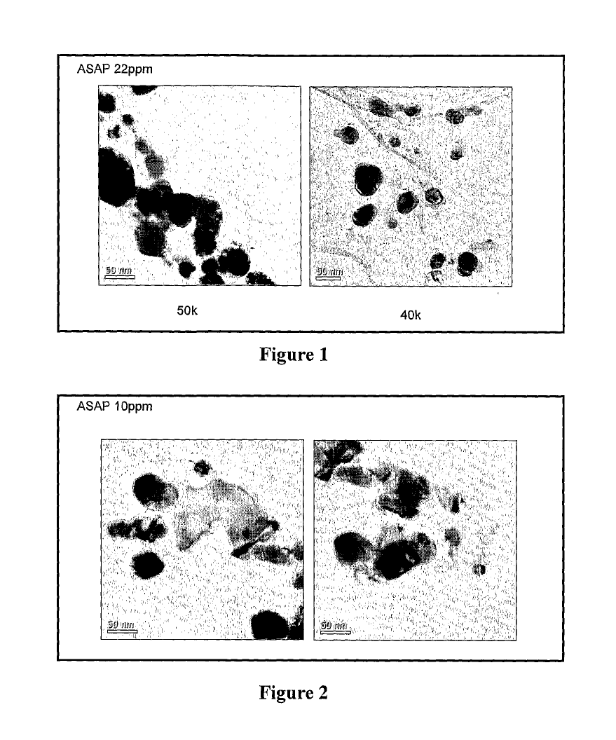

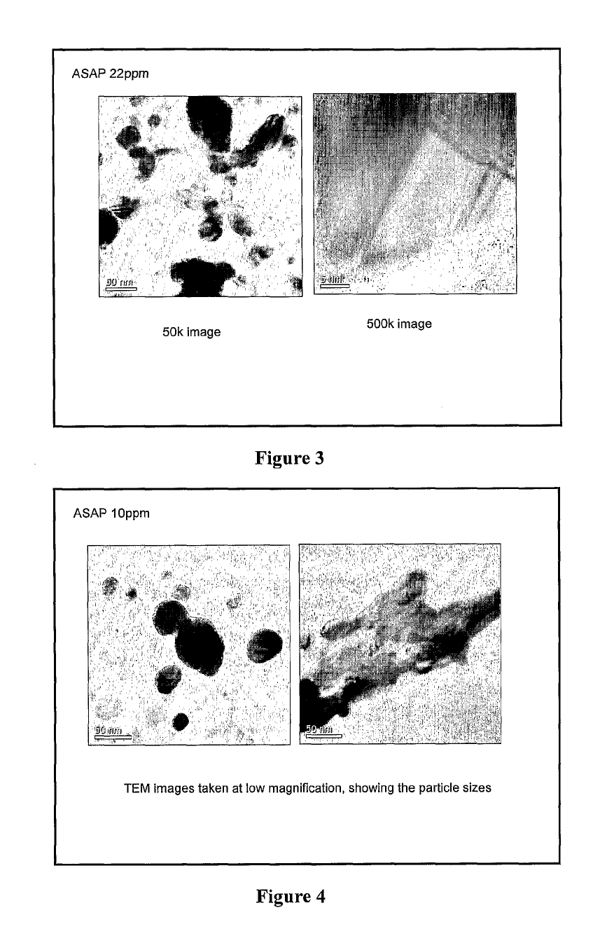

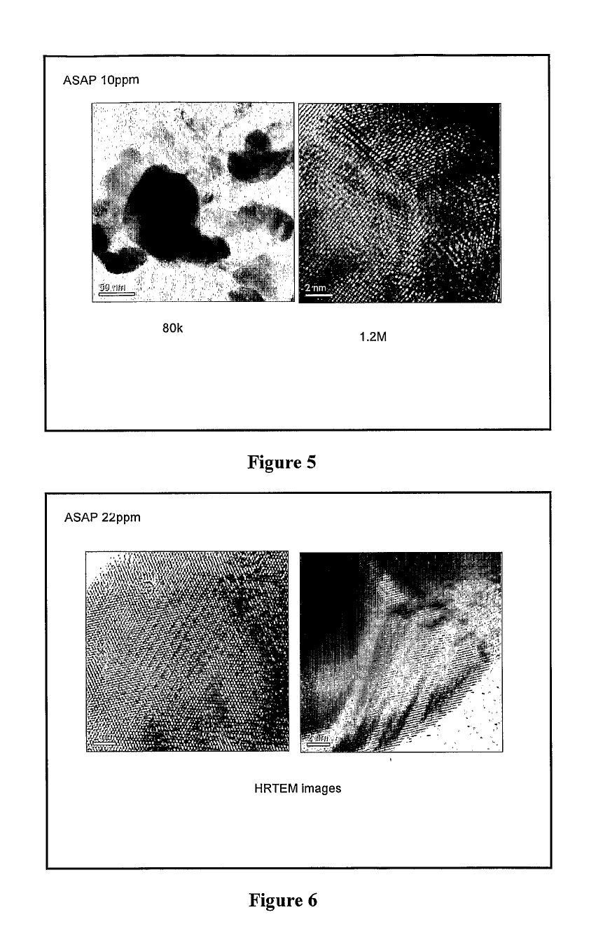

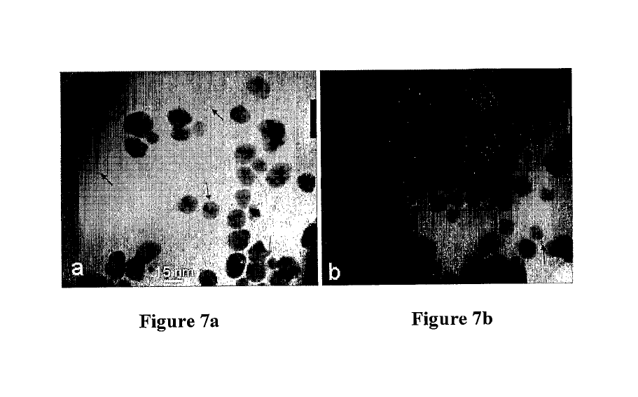

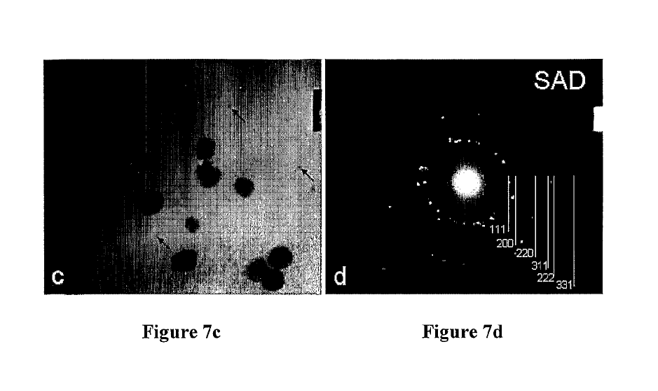

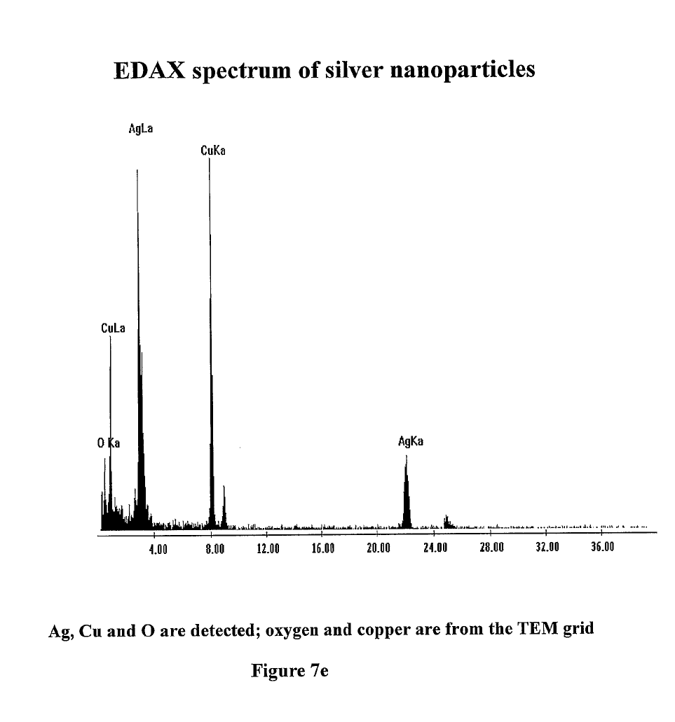

Further particle analysis was performed by SEM, EDS (EDAX) and TEM. In particular, the silver/water compositions were dried and placed on an EM grid and examined in an SEM (i.e., Scanning Electron Microscope) and two different TEMs (i.e., Transmission Electron Microscopes). These analytical tools resulted in determination of particle size distribution in the range of 10-30 nm. However, some estimation of particle size was necessary in some of the generated photomicrographs because the particles tended to clump together or agglomerate on drying. The size of the dried agglomerates was between 50-100 nm. FIGS. 1-6 show various TEM photomicrographs of silver particles dried from the silver/water compositions of the present invention. FIGS. 7a-7d show various TEM photomicrographs of silver particles made according to the present invention, wherein these photomicrographs were generated by a different technique. In particular, the silver/water compositions of the present invention were placed onto C-film and examined by a cryo-TEM (i.e., a different TEM than the TEM used to generate FIGS. 1-6), at a temperature of about −100° C. The silver/water composition of the present invention was therefore substantially instantly frozen. The cryo-TEM was operated at about −100° C. and at a power level of approximately 100kV, and photomicrographs generated are shown in FIGS. 7a, 7b, and 7c. These FIGS. 7a-7c clearly show that the average particle size is less than 20 nanometers. In addition, FIG. 7d shows the TEM analysis in the “SAD” mode. In general, these TEM photomicrographs (FIGS. 7a-7c) show maximum particle sizes of non-clustered silver particles being 15 nanometers or less, and some smaller particles in the 3.5-5 nanometer range. The diffraction analysis shown in FIG. 7d indicates that the particles are primarily metallic silver, are multiply twinned, and are substantially pure. There is a suggestion in these photomicrographs of a possible covering or coating. FIG. 7e shows an EDAX spectrum (i.e., an Energy Dispersion Spectrum or “EDS”) of silver particles taken from silver/water compositions of the present invention. FIG. 7e shows no metallic contaminants at all (e.g., Au, Pt, etc) in the silver. The copper present is from required microscope equipment. There is evidence of a significant amount of oxygen present, which may be present in the copper, as well as being present as coating(s) on a least a portion of the silver particles.

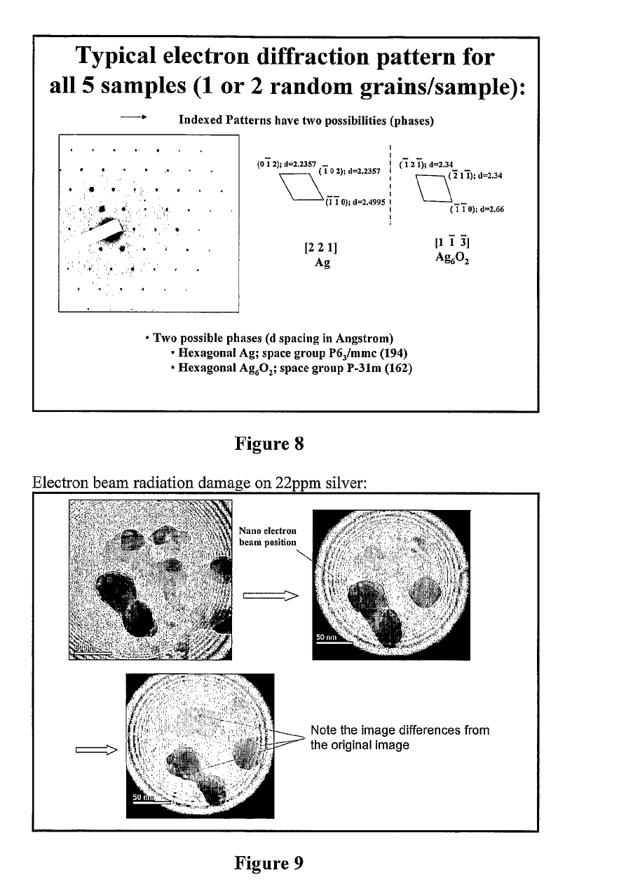

FIG. 8 shows an electron diffraction pattern taken from a silver particle from the present invention. This data suggests the presence of at least one silver oxide species. This data is subject to some interpretation, however, because FIG. 9 shows, for example, possible electron beam damage occurring to silver particles during the data collection process. This electron beam damage was not as evident when examining colloidal silver produced by other manufacturers (discussed later herein). Thus, data collection using SEM and TEM techniques is quite difficult because the energy from the electron beams is capable of damaging (and thus altering) any surface compositions of interest. Thus, great care was taken in generating and analyzing these results.

FIG. 42 shows the results of yet another characterization tool. In this case, powder x-ray diffraction techniques were utilized in an attempt to further demonstrate the existence of oxide phase(s). In particular, FIG. 42 shows four x-ray diffraction patterns taken from four different locations on a dried 200 ppm silver/water composition made according to the present invention. Moreover, superimposed on the four x-ray diffraction patterns are four reference diffraction patterns of species other than pure silver metal. In particular, a 32 ppm silver/water composition made according to the present invention was concentrated to about 200 ppm by a standard reverse-osmosis water filtering process. In particular, the inventive silver/water composition was run through a reverse-osmosis filtering system wherein the “waste” water from the reverse-osmosis filtering system comprised a much more concentrated silver component. Once a 200 ppm solution was obtained, this solution was dried in a flowing nitrogen environment in order to produce a powder which could be subjected to x-ray diffraction. Specifically, the silver/water mixture was placed into pan, the pan was covered with a plastic sheet and nitrogen was introduced into one end of the pan/plastic sheet assembly; and nitrogen exhausted from the opposite end of the pan/plastic sheet assembly. The temperature of the apparatus did not exceed about 75-80° C. in order to maintain the integrity of all components in the silver/water mixture. A sufficient amount of dried powder (i.e., made from the 200 ppm solution) was then available for x-ray diffraction analysis.

The generated x-ray diffraction patterns clearly show the presence of at least four separate species. In this regard, it is clear that a set of silver carbonate peaks occur around 18-22 degrees. These peaks are most likely due to the drying procedure. In this regard, CO₂ in air was most likely present even though attempts were made to create a nitrogen blanket over the 200 ppm solution during the drying procedure. Moreover, a set of peaks occur around 33 degrees. However, each of these peaks could be attributable to silver oxide (AgO), silver carbonate (Ag₂CO₃), and/or silver oxide (Ag₂O). Thus, which species is present is not completely clear. Further, a strong silver metal peak occurs at about 38 degrees. This strong peak can be seen in each of the x-ray diffraction patterns. It is noted, however, that a small silver oxide (Ag₂O) peak also occurs around 38 degrees. Still further, a strong silver oxide (AgO) peak occurs around 37 degrees, in combination with a relatively strong silver carbonate (Ag₂CO₃) peak as well. It is further noted that the silver oxide (AgO) peak corresponds to one of the tetragonal phases of silver oxide. What is clear from reviewing the generated x-ray diffraction data and comparing the same to existing data base files is that one or more oxide phases of silver are present in the inventive silver/water compositions according to the present invention. It is possible that a combination of oxides is present due to the novel processing techniques according to the present invention. It is noted that no x-ray diffraction patterns were available for Ag₄O₄to compare against the x-ray diffraction patterns of the present invention.

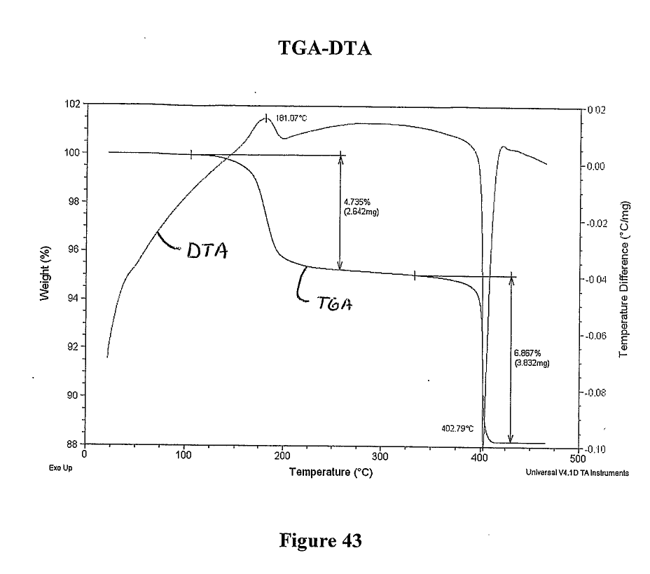

However, Ag₄O₄ does exist commercially. In this regard, a sample of Ag₄O₄ was commercially obtained and a TGA and DTA analysis of such powder was performed. In particular, FIG. 43 corresponds to a TGA analysis and a DTA analysis, respectively. It is clear from the DTA curve in FIG. 43 that an endotherm for Ag₄O₄ exists around 181° C. This endotherm also corresponds to a weight loss shown in the TGA curve of FIG. 43. These experimental measurements correspond to Ag₄O₄ decomposing to Ag₂O. A second very strong endotherm is shown at about 403° C., as well as a second corresponding loss in weight. These two experimental points correspond to Ag₂O decomposing to Ag metal.

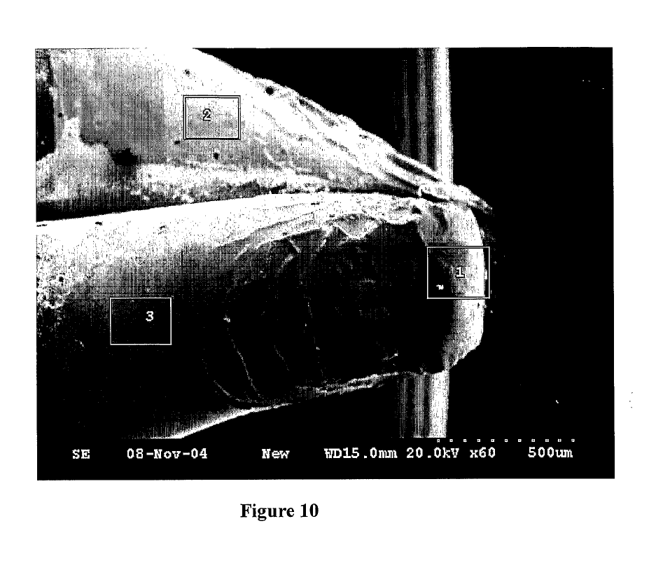

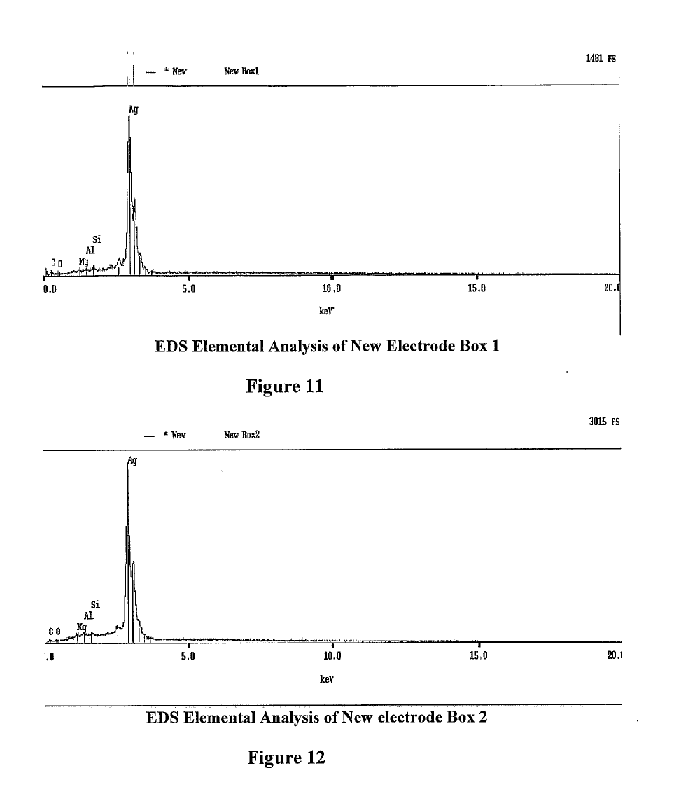

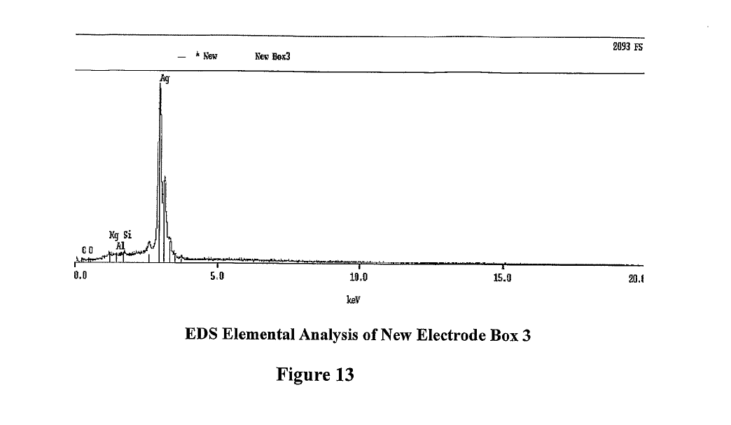

FIG. 10 shows an SEM photomicrograph of a new silver electrode prior to being used in the process according to the present invention. An EDS elemental analysis was performed upon the portions of the electrode labeled as 1, 2 and 3. These three separate analyses appear in FIGS. 11, 12 and 13, respectively. These analyses showed essentially pure silver being present.

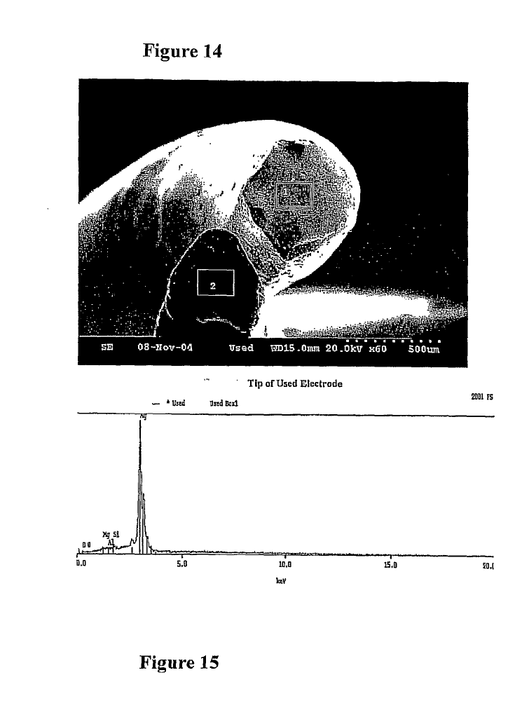

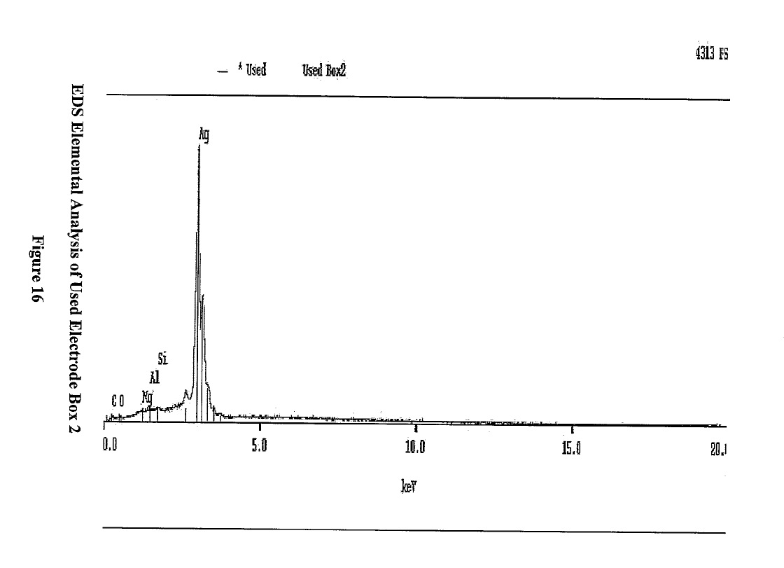

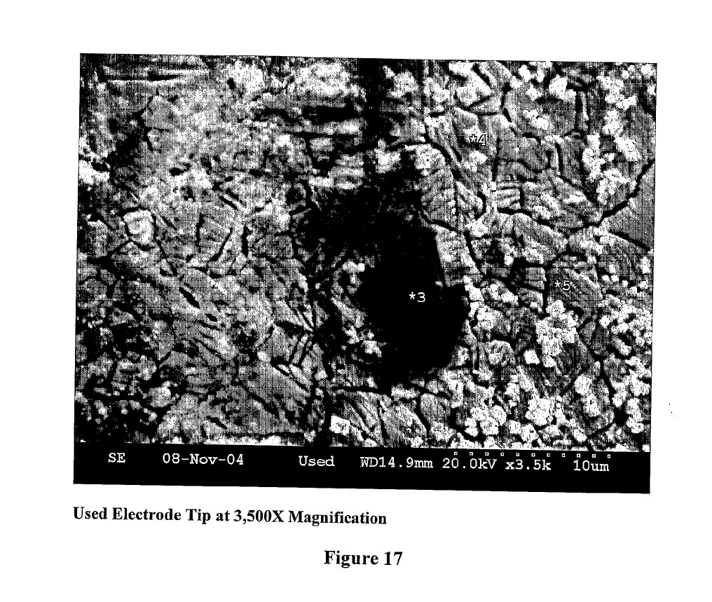

FIG. 14 shows an SEM photomicrograph of the tip of a used silver electrode after it was used in the process according to the present invention. And EDS elemental analysis was performed upon the portions of the used electrode labeled as 1 and 2. These two separate analyses appear in FIGS. 15 and 16, respectively. FIG. 17 shows an SEM photomicrograph of the used electrode tip at a greater magnification (approximately 3500×). The portions 4 and 5 were also examined by EDS elemental analysis and were also found to be substantially pure silver.

Comparison of Silver Particles from Commercially Available Colloidal Silvers

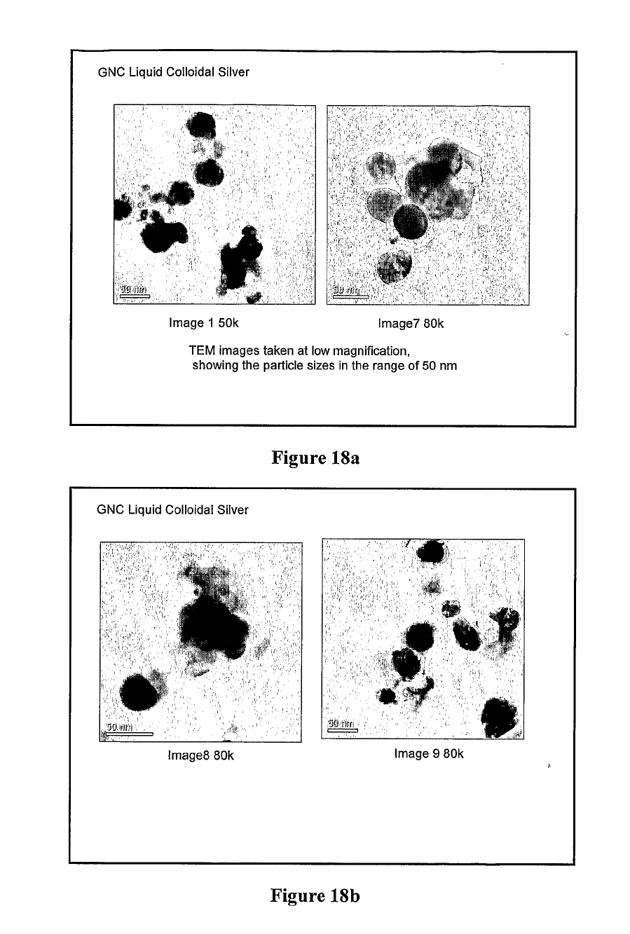

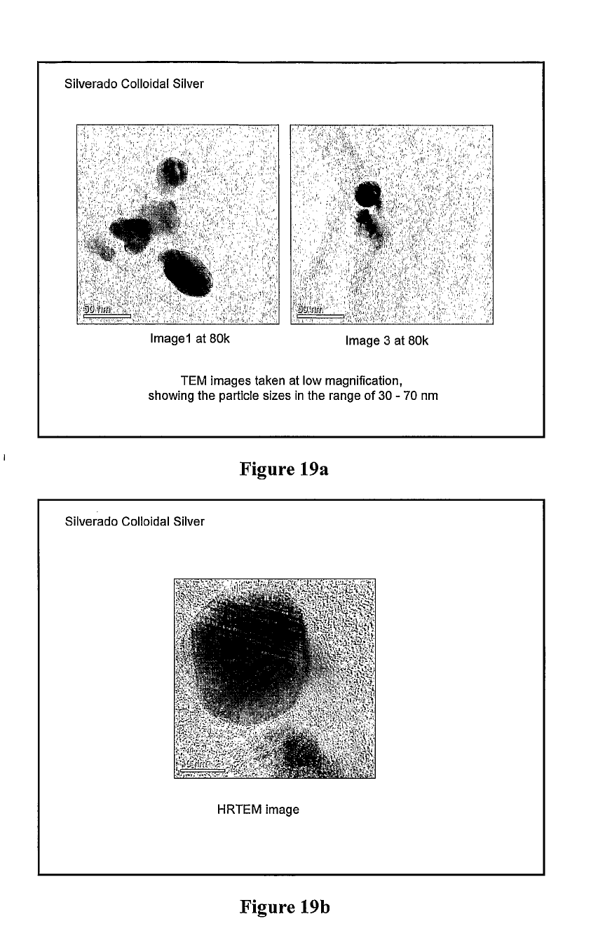

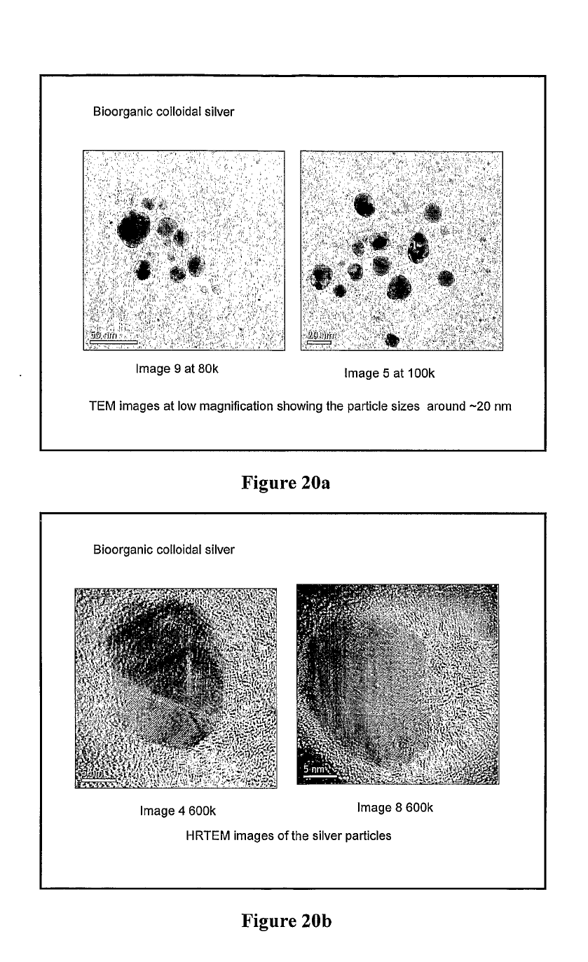

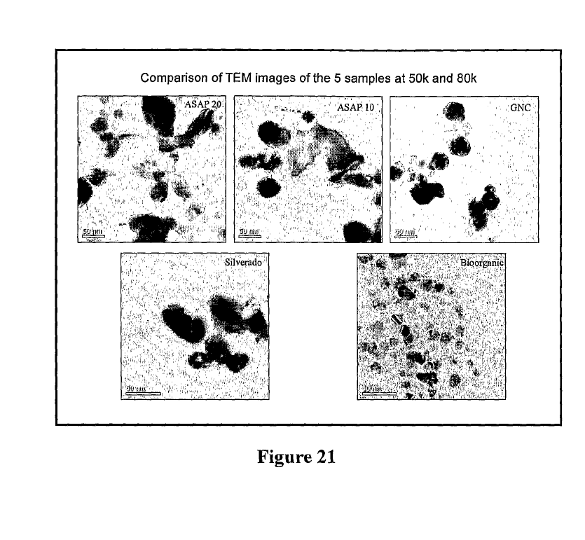

In an effort to understand the differences in performance (e.g., biological efficacy) of silver/water compositions of the present invention, as compared to known colloidal silvers, differences in the physical properties were examined. FIGS. 18a and 18b are TEM photomicrographs of silver particles which correspond to silver particles in a first colloidal silver obtained from General Nutrition Center in 2004 and known in the marketplace as GNC Liquid Colloidal Silver Dietary Supplement (25 ppm) (“GNC”). FIGS. 19a and 19b are TEM photomicrographs of silver particles which correspond to a second colloidal silver known in the marketplace as “Silverado”. FIGS. 20a and 20b are TEM photomicrographs of silver particles which correspond to a third colloidal silver known in the marketplace as Vitamin World Bioorganic Advanced Colloidal Minerals (3 ppm) (Bioorganic). FIG. 21 is an overlay comparison TEM photomicrograph of silver particles from two silver/water inventive compositions (labeled as “ASAP 20” and “ASAP 10”) and from the three known marketplace colloidal silvers known as “GNC”, “Silverado” and “Bioorganic”, discussed above. Clear differences in particle sizes and shapes are evident from these photomicrographs, thus showing that there are physical, structural and potential chemical differences between different colloidal silvers, which may assist in partially explaining the differences in biological efficacy between different products of similar general chemistry.

Spectroscopy Characterization

Raman Spectroscopy

Further analysis of the silver/water mixtures were performed by Raman spectroscopy. A number of analytical approaches on three different Raman spectrometers were performed. The reason for utilizing Raman and Resonance Raman spectroscopy was the belief that different modes (and/or amplitudes) of vibration might be noticeable in the different colloidal silvers when compared to the silver/water compositions of the present invention, as well as compared to “pure” or deionized water. Further, these different observed modes of vibration in the water molecules may help to better define the colloidal systems and to explain the differing biological efficacy in different silver-based products.

In a first set of Raman spectroscopy measurements, a Confocal Raman microscope from Vitech (Ulm, Germany) was used. The model number was CRM200. The spectra were obtained by using a Nikon 60× immersion lens (NA=1) with an integration time per spectrum of 15 seconds (i.e., three separate 5 second acquisition times). The CCD was centered at around 1,799 wavenumbers. A droplet of solution was placed in a small well in a petri dish and the immersion lens was lowered therein. The laser source for the Raman was 532 nm with about 10 mW. The confocal detection system was used with the confocal volume being about 0.3×0.3×0.75 micrometers (approximately 7×10E−8 picoliters).

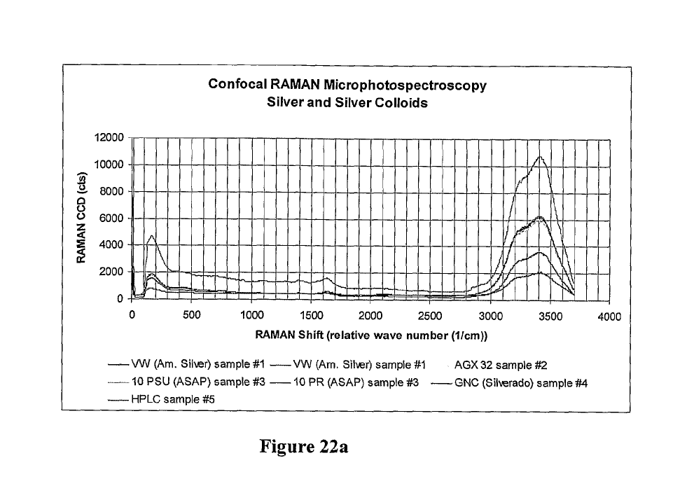

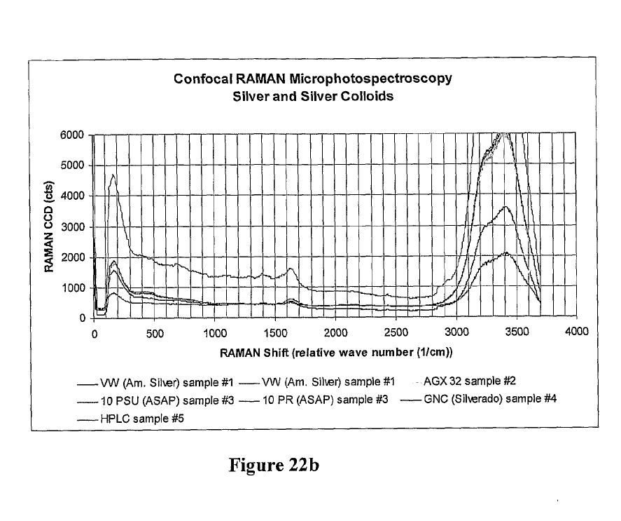

FIGS. 22a and 22b show the graphed results of data collected for 7 samples. Two of the samples were the same, even though labeled differently (10PR and 10 PSU) and correspond to the previously mentioned “ASAP 10” (i.e.,10 ppm silver from the inventive silver/water composition). “HPLC” corresponded to High purity (Ultrapure grade HPLC) water obtained from Alfa Aesar. “DI” corresponded to deionized water. “GNC” corresponded to GNC Liquid Colloidal Silver Dietary Supplement (25 ppm). “AGX-32” corresponded to a 32 ppm inventive silver/water composition. “VW” corresponded to Vitamin World Bioorganic Advanced Colloidal Minerals (3 ppm) (previously referred to as Bioorganic). Clear differences are shown between the different samples. For example, the primary stretching mode (e.g., wavenumbers around 3400-3500 1/cm) in these several water/water-based solutions show large differences. Further, vibrational/rotational behaviors below 500 1/cm also show clear differences between the samples. Some differences can also be seen in the bending modes around 1600 1/cm. Without wishing to be bound by any particular theory or explanation, it appears as though the different behaviors of the silver/water compositions of the present invention may, for example, influence, or at least help to explain, the efficacy of such compositions relative to the other samples investigated.

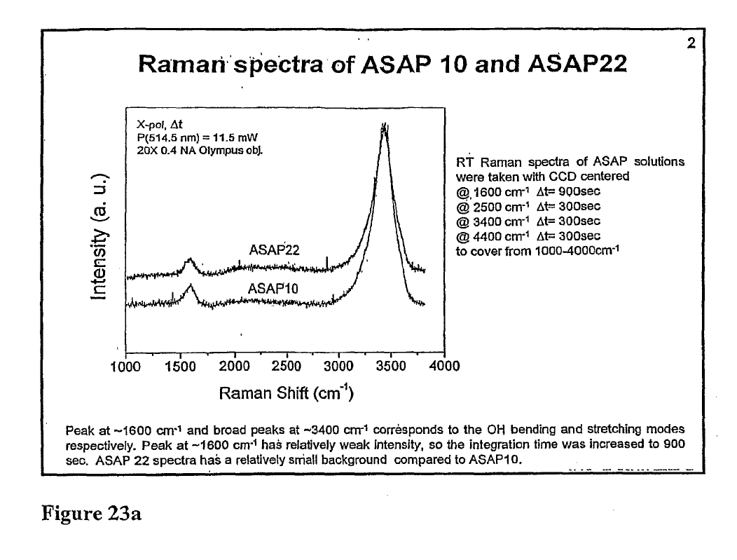

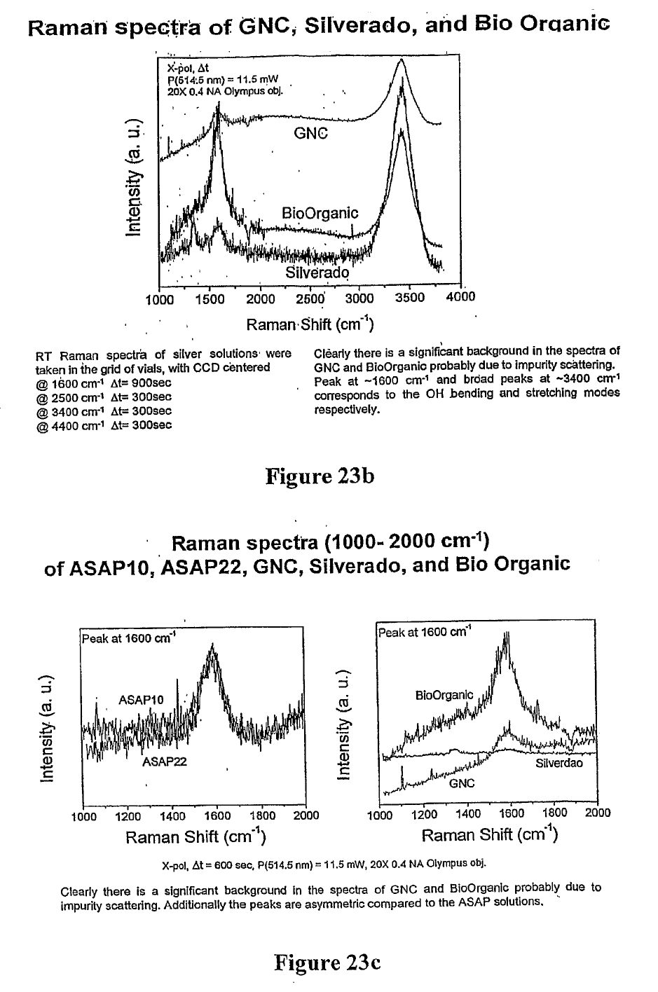

A second set of Raman data was generated from a different spectrographic system. While the numbers generated between the two sets of data are different (which strongly suggests that Raman spectroscopy data for water is a function of the analytical device used) the data within this data set also shows remarkable differences between silver/water compositions of the present invention when compared to other colloidal silvers or other waters. In this set of Raman spectroscopy measurements, a reflection Raman microscope was used. The spectra were obtained by using an Olympus 20× lens (NA=0.4). The CCD detector was centered at four different wavenumbers, namely, 1600, 2500, 3400 and 4400 1/cm. The laser source for the Raman was 514.5 nm with about 11.5 mW. Additional information regarding the spectra can be found on each of the FIGS. 23a, 23b and 23c. The labeling of the samples on these Figures is consistent with the text above. Both sets of Raman spectroscopy data strongly suggest that different molecular movement exists within these different samples which may be contributing to (or at least evidencing) the biological effectiveness of the silver/water compositions according to the present invention.

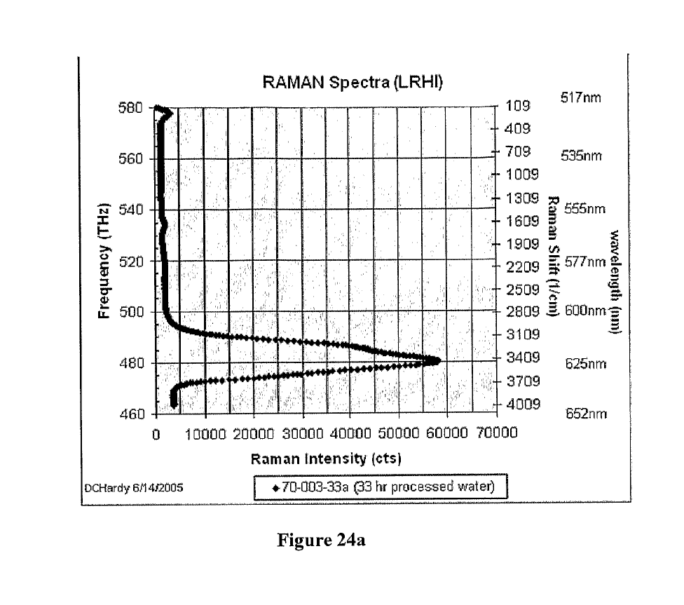

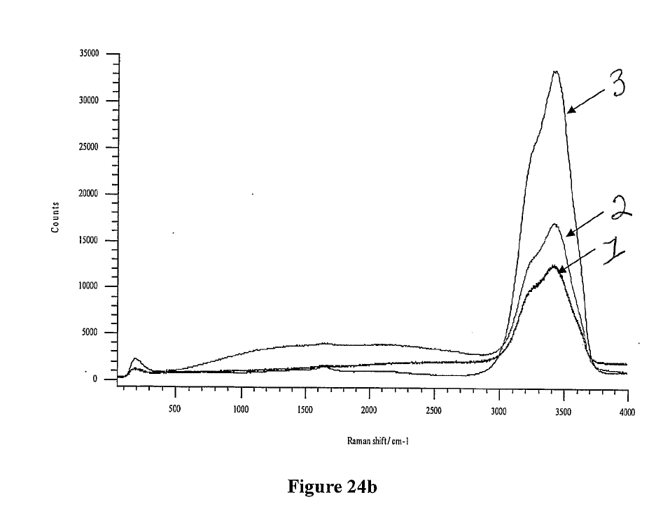

A third set of Raman data was generated using a third multiple laser-line Renishaw Confocal Raman Micro-spectrograph. This system was configured to permit measurements both above and immersed within the sample. The setup was designed to investigate a 100× to 1000× larger sample volume than that described in the first set of measurements. The reflection micro-spectrograph with Leica DL DM microscope was fitted with either a 20× (NA=0.5) water immersion or a 5× (NA=0.12) dry lens. The rear aperture of each lens was sized to equal or exceed the expanded laser beam diameter. Two laser frequencies were used, these being a multiline 50 mW Argon laser at ½ power setup for 514.5 nm and a 20 mW HeNe laser at 633 nm. High resolution gratings were fitted in the monochrometer optic path which allowed continuous scans from 50 to 4000 wavenumbers (1/cm). Ten to 20 second integration times were used. Sample fluid was placed below the lens in a 50 ml beaker. Both lasers were used to investigate resonance bands, while the former laser was primarily used to obtain Raman spectra. Sample size was about 25 ml. Measurements made with the 5× dry lens were made with the objective positioned about 5 mm above the fluid to interrogate a volume about 7 mm beneath the water meniscus. Immersion measurements were made with the 20× immersion lens positioned about 4 mm into the sample allowing investigation of the same spatial volume. CCD detector acquisition areas were individually adjusted for each lens to maximize signal intensity and signal-to-noise ratios. A representative spectra for silver/water compositions of the present invention is shown in FIG. 24a. FIG. 24b shows the Raman Spectra of three different metal/water solutions made according to the present invention. Plot 1 corresponds to a 13 ppm silver/water solution; Plot 2 corresponds to a 10 ppm zinc/water solution; and Plot 3 corresponds to an 11 ppm copper/water solution.

While the numbers generated between the three sets of data are somewhat different (which strongly suggest that Raman spectroscopy data for water is a function of the analytical instrument and setup of that instrument), the data within these data sets demonstrate remarkable differences between silver/water compositions of the present invention when compared to other colloidal silvers or other waters. All sets of Raman spectroscopy data strongly suggest that different molecular movement and bonds exist within these difference samples which may be contributing to (or at least evidencing) the effectiveness of the silver/water compositions according to the present invention. Further, differences in Raman patterns for the three different metal/water solutions shown in FIG. 24b also suggests possible differing effectiveness.

UV-Vis Spectroscopy

Further analysis of the silver/water mixtures were performed by UV-Vis spectroscopy. UV-Vis spectroscopy was utilized in addition to Raman spectroscopy to search for additional distinguishing modes and/or amplitudes of vibration in a different part of the spectrum. A single UV-Vis spectrometer was utilized to collect the data. In this regard, energy absorption spectra were obtained using UV-Vis micro-spec-photometry. This information was acquired using dual beam scanning monochrometer systems capable of scanning the wavelength range of about 190 nm to about 1100 nm. The UV-Vis spectrometer that was used to collect absorption spectra was a Jasco MSV350. The instrument was set up to support measurement of low-concentration liquid samples using a 10 mm×10 mm fuzed quartz cuvette. Data was acquired over the above wavelength range using both a photo multiplier tube (PMT) and a Photo Diode detector with the following operational parameters: a bandwidth collection of 2 nm, a resolution of 0.5 nm; and a water baseline background subtracted from the generated spectra. In this regard, the UV-Vis signature for pure water was subtracted from the generated spectra so as to show more representative spectral signatures for the silver/water mixture.

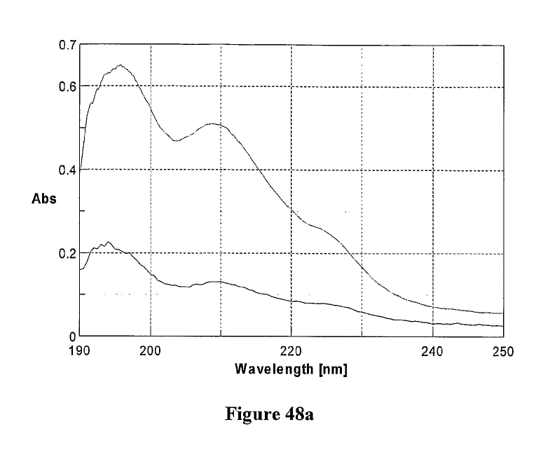

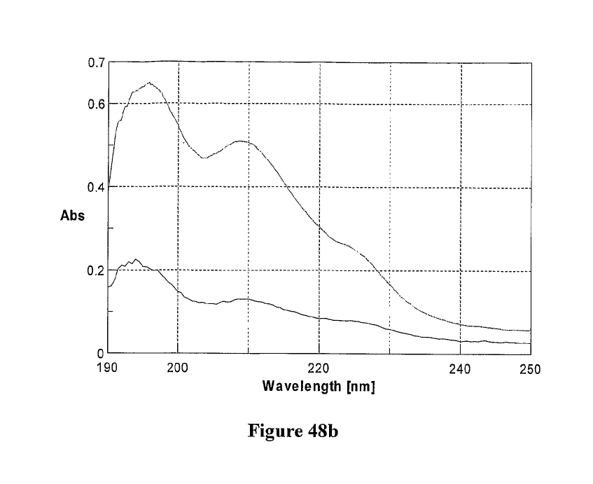

Both Tungsten “halogen” and Hydrogen “D2” energy sources were used as the primary energy sources for the MSV350. The optical path of the spectrometer was set to allow the energy beam to pass through the samples with focus towards the center of the sample cuvettes. Sample preparation was limited to filling and capping the cuvettes and physically setting them onto the cuvette holder within the fully enclosed sample compartment. Data output was measured and displayed as Absorbance Units (per Beer-Lambert's Law) versus wavelength and frequency. The primary difference between the samples corresponding to the two spectra shown in each of FIGS. 48a and 48b was the silver concentration of silver in each of the samples. Specifically, the higher amplitude curve in each of FIGS. 48a and 48b correspond to a 32 ppm silver/water solution; and the lower amplitude curve corresponds to a 10 ppm silver/water solution. The wavelength or frequency positions of the peaks (i.e., the locations of the peaks and dips) are quite similar.

As discussed above herein, silver (e.g., silver ions, silver metals, Ag+, etc.) can be controllably attached or fixed, for example, between and/or on clay layers and/or within cages of zeolites. One method of achieving placement of, for example, silver ions into or onto clays, micas, or zeolites, is to provide an ionic species of silver in a soluble state and introduce said species into a clay or zeolite composition or mixture. The concept of exchanging, for example, a silver ion for another positively charged ion is sometimes referred to as “BEC” or “CEC” (these are both shorthand nomenclatures for referring to cation exchange capacity of a “system”). In this regard, most kaolinite materials are known to have cation exchange capacities which are in the range of 2-5 (i.e., 2-5 meq/100 grams). Montmorillonite clays, for example, have cation exchange capacities around 100 meq/100 grams. Whereas, zeolites can have cation exchange capacities of several hundred meqs/100 grams. For example, a well known zeolite known as “Linde 4A zeolite” is 400-500 meq/100 grams for its BEC or CEC number. In general, the higher the BEG or CEC number, the greater the ability for the material to receive cations.

Experimental procedures to determine whether or not kaolinites or zeolites are capable of becoming a silver (or other metal cation(s)) holder/delivery systems were conducted. In particular, the following steps were used to prepare and therefore analyze silver-clay samples, as well as silver-zeolite samples.

In general terms, typical kaolinite and zeolite Linde 4A materials were first washed three times with deionized water to remove possible chlorine contamination which might cause certain silver starting materials (e.g., silver ions) to precipitate (or undesirably react) before the silver starting materials could desirably attach onto/into the kaolinite and/or zeolite structures. These washed materials were then mixed with a silver nitrate (AgNO₃) solution in appropriate concentrations corresponding to the expected or known “CEC” of each respective material. The resulting treated materials were then again washed with deionized water to remove any unused silver nitrate. The samples were dried overnight in an electric-resistance drying oven at about 120° C. In particular, the washing procedure was as follows:

About two grams of each kaolinite or zeolite sample was placed in a centrifuge tube. Deionized water was then added. Sample and deionized water mixture was then agitated on a wrist shaker for about 40 minutes. The mixture was then centrifuged for about 30 minutes at about 1000 RPM. Excess liquid was then decanted from the sample tube. The steps of adding deionized water, shaking, centrifuging, and decanting was repeated for a total of three washes.

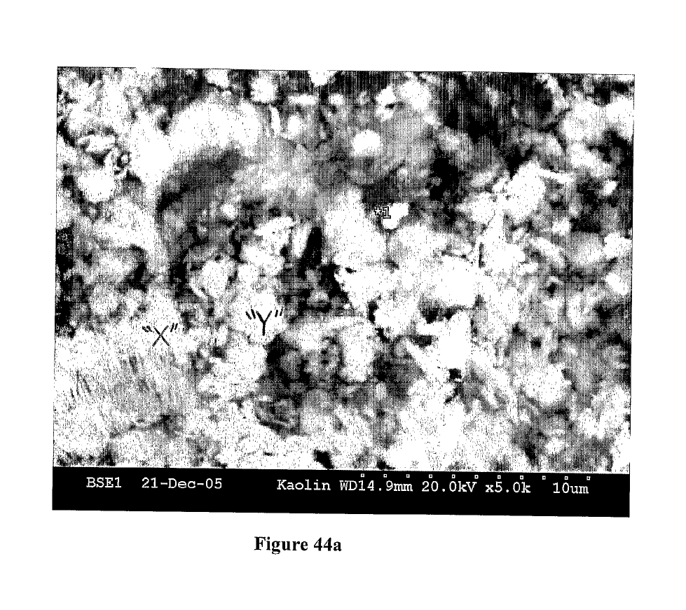

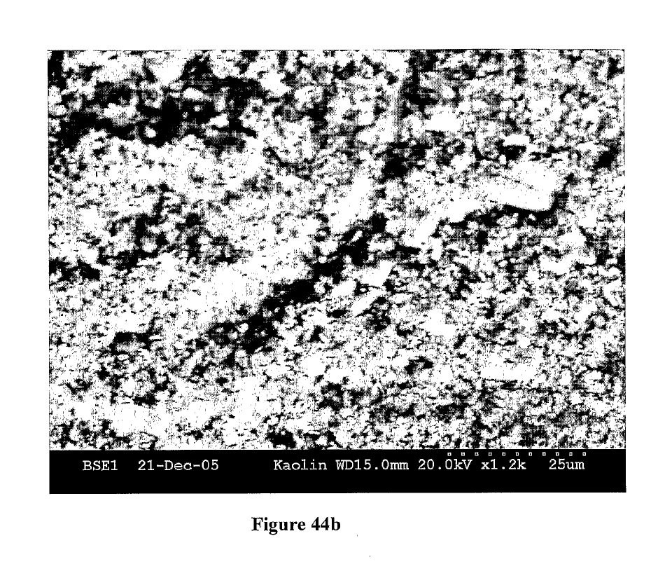

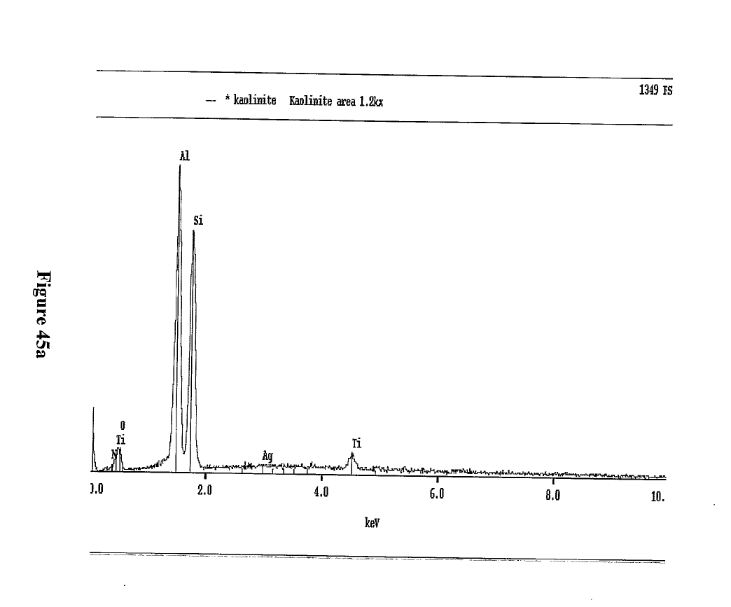

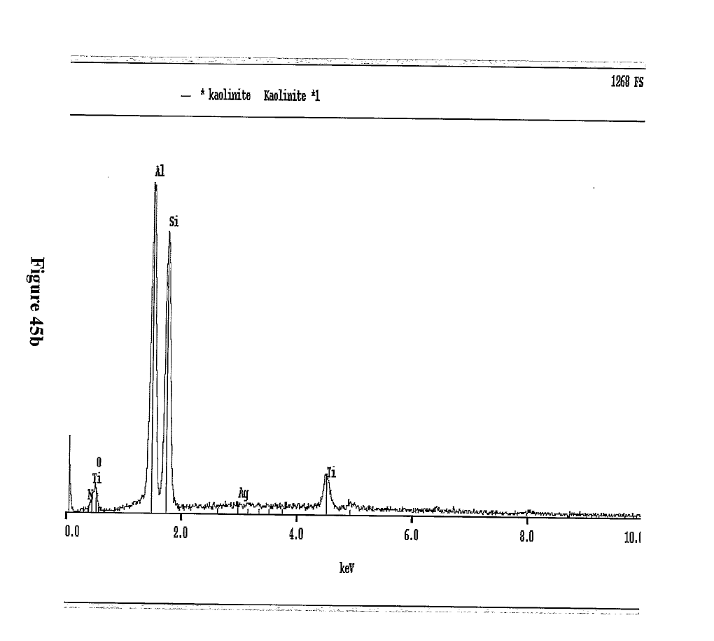

Once the initial 2 gram sample had been appropriately washed, so as to remove possible chlorine contamination, silver nitrate was introduced into the cleaned kaolinite and zeolite materials. In particular, about 0.09 grams of silver nitrate was introduced into the kaolinite mixture and about 4.25 grams of AgNO₃ was introduced to the zeolite Linde 4A. In particular, the measured amounts of silver nitrate were added to each tube, deionized water was then added to fill the tube, the mixture was then agitated on a wrist shaker for about 40 minutes, and thereafter, centrifuged for about 30 minutes at about 1000 RPM. Liquid was then decanted. This procedure of adding silver nitrate, adding deionized water, agitating on a wrist shaker, centrifuging, and decanting was repeated for a total of three times. After the washing and silver nitrate introduction procedures had been completed, the samples were removed from the centrifuge tube and placed into an aluminum (Al₂O₃) crucible and dried overnight at about 120° C. in an electrical resistance heated furnace. The resultant kaolinite/silver and zeolite/silver materials were then characterized by SEM photomicrographs and SEM EDS (EDAX) techniques. FIGS. 44a and 44b show SEM photomicrographs of the kaolinite samples made according to techniques discussed above herein. It is clear from these photomicrographs that the “book-like” or “sheet-like” structures of the kaolinites (e.g., layers of SiO₂ and AlO₂O₃ identified as “X” and “Y” in FIG. 44a) show clearly that silver cations have been located around the “edges” of the clay materials. Clearly there has been some type of silver attachment or exchange, as evidenced by the brighter “page-like” portions of these photomicrographs (Note: the portions “X” and “Y” are representative of various other “book-like” structures in the sample). FIGS. 45a-45b show EDS (EDAX) analysis of the samples shown in FIGS. 45a and 45b, respectively. These analyses clearly show the presence of aluminum and silicon, as would be expected for kaolinite, as well as some titanium (suggesting the presence of rutile). Very small peaks of silver can also be seen, which correspond to BEC numbers for kaolin being relatively low at 2-5.

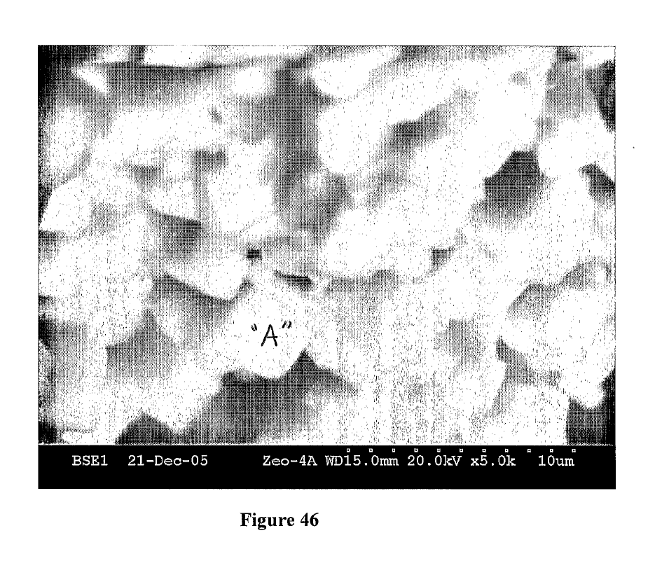

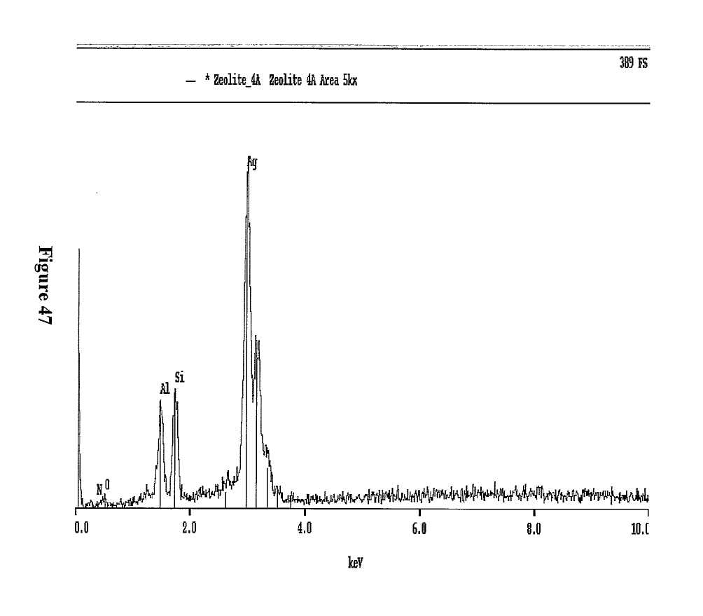

FIG. 46 shows an SEM photomicrograph corresponding to zeolites processed according to the procedures discussed above herein. Due to the higher CEC number of zeolite (namely, about 500) the zeolite cube-like structures in FIG. 46 appear to be “glowing” in the photomicrograph (see, for example, the portion “A” in FIG. 46). This “glowing” suggests that there has been a substantial even distribution of silver in and throughout the zeolite structures. In this regard, if there were bright spots of silver metal by itself glowing brightly, then the silver would not have been incorporated into/onto the zeolite. FIG. 47 is an EDS (EDAX) analysis of the sample shown in FIG. 46. Again, relatively high amplitude peaks of aluminum and silicon are present, but extremely high peaks of silver are present (i.e., in comparison to the Ag peaks in the kaolinite shown in FIGS. 45a-45b). These very high silver peaks correspond to the much greater ability of the zeolite to capture silver in its structure (i.e., a high BEC) relative to the structure of the kaolinites (i.e., a low BEC) shown in FIGS. 44a and 44b.

Evidence of Efficacy of 22 ppm Silver Composition Against Bacilus Subtilis

A. Purpose of Example

The purpose of this example is to demonstrate the antimicrobial activity of the silver-based composition of the present invention on bacterial endospores from the test organism Bacillus subtilis. This was accomplished by performing a standard kill-time assay using a suspension of B. subtilis endospores. Normally, bacterial endospores are resistant to killing.

B. Material and Methods

Test Organism. A test suspension containing endospores from Bacillus subtilis (ATTC #19659) was prepared from a culture grown on nutrient agar, to which additional sporulation enhancement ingredients were added. Plates were harvested with sterile water and endospores were purified by repeated centrifugations and resuspensions in water. The final wash was in 70% ethanol for 30 min, to ensure the destruction of all vegetative bacteria. The spores were resuspended in water containing 0.1% Tween 80 (brand of polysorbate surfactant) to prevent clumping.

Neutralizer. The Neutralizer mixture consisted of 12.7% Tween® 80 (brand of polysorbate), 6.0% Tamol® SN (brand of sodium salt of naphthalene-formaldehyde condensate), 1.7% lecithin, 1% Peptone, and 0.1% Cystine. This solution was intended to neutralize any chemicals so they would not affect subsequent growth of the bacteria.

Kill-Time Procedure:

a) A 9.9 ml aliquot of the disinfectant (inventive 22 ppm silver composition, in water) was placed in a sterile 20 mm×150 mm tube. The tube was equilibrated in a 20° C. water bath.

b) A 9.9 ml aliquot of the disinfectant (inventive 22 ppm silver composition, in water) was placed in a sterile 20 mm×150 mm tube. The tube was equilibrated in a 20° C. water bath.

c) At 30 mins., 1 hr, and 4 hr, one ml of organism/disinfectant suspension was removed to a tube containing nine ml of Neutralizer. The tube was mixed thoroughly.

d) After two min, the neutralized suspension was serially diluted 1:10, in physiological saline solution (PSS).

e) The number of viable organisms in selected dilution tubes was assayed by membrane filtration. One ml aliquots were plated in duplicate. The membranes were washed with about 100 ml of sterile PSS and removed to Nutrient Agar plates. The plates were incubated at 37° C. for 20 hr.

f) The number of colonies on each filter was counted and log reductions were computed.

Controls:

a) Titers of the test suspensions were computed by performing membrane filtration assays of selected 1:10 dilutions of the test suspensions in PSS.

b) A neutralizer control was performed by inoculating a mixture of 9 ml neutralizer and 1 ml of disinfectant with 100 ml of a dilution of the titer containing 100 cfu. This produced about 10 cfu/ml in the tube, which was allowed to stand for 20 minutes prior to assay by membrane filtration using duplicate 1 ml samples.

C. Results

Bacillus Subtilis Titer:

| | Dilution: | | |
|---|---|---|---|
| | 1:1 × 10⁶ | 1:1 × 10⁷ | 1:1 × 10⁸ |
| Number of colonies: | TNTC | 75 | 7 |
| | TNTC | 58 | 8 |

TNTC = too numerous to count

### Dilution of B. Subtilus Spore/Disinfectant Suspension:

| | 1:1 × | 1:1 × | 1:1 × | 1:1 × | 1:1 × | 1:1 × |
|---|---|---|---|---|---|---|
| Time | 10¹ | 10² | 10³ | 10⁴ | 10⁵ | 10⁶ |
| 30 min | — | — | TNTC | TNTC | 57 | 10 |
| | — | — | TNTC | TNTC | 51 | 7 |
| 1 hr | — | — | TNTC | TNTC | 28 | 3 |
| | — | — | TNTC | TNTC | 55 | 3 |
| 2 hr | — | TNTC | TNTC | 126 | 23 | — |
| | — | TNTC | TNTC | 183 | 17 | — |
| 4 hr | TNTC | TNTC | 88 | 12 | — | — |
| | TNTC | TNTC | 69 | 12 | — | — |

TNTC = too numerous to count
Neutralization Control: 1:1 × 10⁸

D. Discussion

Results of the titer showed a viable B. subtilis spore concentration of 6.65×10⁸ spores per ml in the original suspension. Inoculation of 9.9 ml of disinfectant with 100 ml of this suspension produced an initial concentration of 6.65×10⁶ spores per ml in the assay tube.

Results from these procedures allowed log reductions (LR) and Percent Kill (PK) values to be calculated. They are listed in the table below. Values were computed using the formulae: LR=−Log(S/So) and PK=(1−(S/So))×100; where S=concentration of organisms at a specific time; and So=the initial concentration of organisms at time zero.

| | | PERCENT |
|---|---|---|
| Time | LOG REDUCTION | KILL |
| 30 min | 0.090 | 18.8 |
| 1 hr | 0.205 | 37.6 |
| 2 hr | 0.634 | 76.8 |
| 4 hr | 1.928 | 98.8 |

Neutralization control data showed that the disinfectant was adequately neutralized. Actual counts correspond to those resulting from dilution without appreciable killing.

The disinfectant preparation tested here displayed good sporicidal activity against B. subtilis spores. B. subtilis is a common species used in sporicidal testing and belongs to the same genus as the organism that causes anthrax. Because of their genetic similarities, B. subtilis spores have been used as a non-pathogenic surrogate for Bacillus anthracis, the anthrax bacterium. Therefore, these results are applicable to anthrax. It is expected that longer exposure would result in additional killing.

Evidence of Efficacy of 10 ppm Silver and 1.0% H₂0₂ Composition and 14 ppm Silver and 1.5% H₂O₂ Composition Against Bacillus Subtilis

A. Purpose of Example

The purpose of this example is to demonstrate the antimicrobial activity of two silver-based compositions of the present invention on bacterial endospores from the test organism Bacillus subtilis. This was accomplished by performing standard kill-time assays using a suspension of B. subtilis endospores. Viewed relative to the previous example (employing 22 ppm silver), this example establishes the promoting effect of hydrogen peroxide (H₂O₂) on the antimicrobial properties of silver compositions. Hydrogen peroxide is stable in the presence of the silver compositions of the present invention. While hydrogen peroxide has significant antimicrobial properties itself, it is frequently broken down by catalase or other microbial enzymes. However, the hydrogen peroxide is capable of weakening bacterial cell walls and enhancing entry of the silver particles before any enzymatic destruction of the hydrogen peroxide can occur.

B. Material and Methods

1. Test Organism. A test suspension containing endospores from Bacillus subtilis (ATCC #19659) was prepared from a culture grown on Nutrient Agar, to which additional sporulation enhancers were added. Plates were harvested with sterile water and endospores were purified by repeated centrifugations and resuspensions in water. The final wash was in 70% ethanol for 30 min, to ensure the death of all vegetative bacteria. The spores were resuspended in water containing 0.1% Tween® 80 (brand of polysorbate) to prevent clumping.

2. Neutralizer. The Neutralizer mixture consisted of 12.7% Tween 80, 6.0% Tamol® SN (brand of sodium salt of naphthalene-formaldehyde condensate), 1.7% lecithin, 1% Peptone, and 0.1% Cystine. This solution was intended to neutralize any chemicals so they would not affect subsequent growth of the bacteria.

3. Kill-Time Procedure:

a) A 9.9 ml aliquot of each of the disinfectants (inventive colloidal silver compositions: one containing 14 ppm silver and 1.5% H202; the other containing 10 ppm silver and 1.0% H₂O₂) was placed in a sterile 20 mm×150 mm tube. The tubes were equilibrated in a 20° C. water bath.

b) Each tube of disinfectant was inoculated with 100 ml of the test organism suspension at time zero.

c) At 10 min, 30 min, 1 hr, 2 hr, 4 hr, 6 hr, and 8 hr, one ml of organism/disinfectant suspension was removed to a tube containing nine ml of neutralizer. The tube was mixed thoroughly.

d) After two min, the neutralized suspension was serially diluted 1:10, in physiological saline solution (PSS).

e) The number of viable organisms in selected dilution tubes was assayed by membrane filtration. One ml aliquots were plated in duplicate. The membranes were washed with about 100 ml of sterile PSS and removed to Columbia Agar plates. The plates were incubated at 37° C. for 20 hr.

f) The number of colonies on each filter was counted and log reductions computed.

4. Controls:

a) Titers of the test suspensions were computed by performing membrane filtration assays of selected 1:10 dilutions of the test suspensions in PSS.

b) A neutralizer control was performed by inoculating a mixture of 9 ml of neutralizer and 1 ml of disinfectant with 100 ml of the 1:10³ dilution of the titer. This produced about 2,000 cfu / ml in the tube, which was allowed to stand for 20 minutes prior to diluting 1:10. Both tubes were assayed by membrane filtration using duplicate 1 ml samples. All results are shown in Tables 1a and 1b.

C. Results

Titer of Bacillus Subtilis Spores:

| | Dilution: | | |
|---|---|---|---|
| | 1:1 × 10⁶ | 1:1 × 10⁷ | 1:1 × 10⁸ |
| Number of colonies: | TNTC | 36 | 5 |
| | TNTC | 27 | 4 |

TNTC = too numerous to count

TABLE 1a
Solution containing 14 ppm silver and 1.5% H₂O₂:
Dilution of B. subtilis spore/disinfectant suspension:

| Time | 1:1 × 10¹ | 1:1 × 10² | 1:1 × 10³ | 1:1 × 10⁴ | 1:1 × 10⁵ |
|---|---|---|---|---|---|
| 10 min | — | — | TNTC | TNTC | 227 |
| | — | — | TNTC | TNTC | 265 |
| 30 min | — | — | TNTC | TNTC | 258 |
| | — | — | TNTC | TNTC | 273 |
| 1 hr | — | — | TNTC | TNTC | 55 |
| | — | — | TNTC | TNTC | 33 |
| 2 hr | — | TNTC | 207 | 29 | — |
| — | — | TNTC | 237 | 24 | — |
| 4 hr | 59 | 3 | 1 | | |
| | 57 | 5 | 1 | | |
| 6 hr | 0 | 0 | 0 | | |
| | 3 | 0 | 0 | | |
| 8 hr | 1 | 0 | 0 | | |
| | 1 | 0 | 0 | | |

TNTC = too numerous to count.

Neutralization Control:

| Undiluted | 1:1 × 10¹ |
|---|---|
| TNTC | 195 |
| TNTC | 210 |

TNTC = too numerous to count.

TABLE 1b
Solution containing 14 ppm silver and 1.5% H₂O₂:
Dilution of B. subtilis spore/disinfectant suspension:

| Time | 1:1 × 10¹ | 1:1 × 10² | 1:1 × 10³ | 1:1 × 10⁴ | 1:1 × 10⁵ |
|---|---|---|---|---|---|
| 10 min | — | — | TNTC | TNTC | 230 |
| | — | — | TNTC | TNTC | 287 |
| 30 min | — | — | TNTC | TNTC | 254 |
| | — | — | TNTC | TNTC | 260 |
| 1 hr | — | — | TNTC | TNTC | 146 |
| | — | — | TNTC | TNTC | 124 |
| 2 hr | — | TNTC | TNTC | 64 | — |
| | — | TNTC | TNTC | 71 | — |
| 4 hr | TNTC | 72 | 5 | | |
| | TNTC | 77 | 5 | | |
| 6 hr | 0 | 0 | 0 | | |
| | 2 | 0 | 0 | | |
| 8 hr | 0 | 0 | 0 | | |
| | 0 | 0 | 0 | | |

TNTC = too numerous to count.

Neutralization Control:

| Undiluted | 1:1 × 10¹ |
|---|---|
| TNTC | 200 |
| TNTC | 184 |

TNTC = too numerous to count.

D. Discussion

The data showed a viable B. subtilis spore concentration of 2.59×10⁸ spores per ml in the original suspension. Inoculation of 9.9 ml of disinfectant with 100 [symbol] l of this suspension produced an initial concentration of 2.59×10⁵ spores per ml in the assay tube.

Results from these procedures allowed log reductions (LR) and Percent Kill (PK) values to be calculated. They are listed in the following table. Values were computed using the formulae: LR=−Log(S/So) and PK=(1−(S/So))×100; where. S=concentration of organisms at a specific time; and So=the initial concentration of organisms at time zero. Since there was no significant kill within 30 min, the 10 min data was used for the So values. The 6 hr and 8 hr exposure times did not produce counts high enough to be reliable. Therefore, these data were not used in the linear regressions. Linear regressions were performed on the log reduction values using the ‘fitted line plots’ command in the Minitab statistical software package. The regression equations produced, and the times required to effect a six-log reduction are shown along with the log reduction and percent kill values in the following Table 2.

TABLE 2

| | 14 ppm SILVER + 1.5% H₂O₂ | | 10 ppm SILVER + 1.0% H₂O₂ | |
|---|---|---|---|---|
| | LOG | PERCENT | LOG | PERCENT |
| Time | REDUCTION | KILL | REDUCTION | KILL |
| 30 min | −0.03 | −7.9 | 0.003 | 0.6 |
| 1 hr | 0.66 | 78.0 | 0.28 | 47.8 |
| 2 hr | 2.05 | 99.1 | 1.58 | 97.4 |
| 4 hr | 4.63 | 99.998 | 3.54 | 99.97 |

Regression Analysis

Equation for 14 ppm calculated line: Y=−0.66704+1.32936x. Equation for 10 ppm calculated line: Y=−0.59690+1.03933x. These equations predict that the time for a 6-log reduction is 5.02 hrs for the 14 ppm composition and 6.35 hrs for the 10 ppm composition.

The neutralization control data showed that the disinfectant was adequately neutralized. Expected counts corresponded to those expected from the dilution.

The experimental disinfectant solutions tested exhibited significant sporicidal activity against B. subtilis spores. The B. subtilis strain used in these evaluations is the same one specified in the AOAC sporicidal test. Spores from this organism represent a significant challenge for most disinfectants. The times required to effect a six log reduction are in line with the sporicidal label claims of many cold sterilants.

Evidence of Efficacy of 10 ppm Silver Composition as a Broad Spectrum Antimicrobial

A. Methods

MIC (minimum inhibitory concentration) and MBC (minimum bactericidal concentration) tests were performed according to the standard broth microdilution method. The MIC is defined as the lowest concentration of an antibiotic that will inhibit the (in vitro) growth of an infectious organism. Results are reported in micrograms per ml. For medical antibiotics the interpretation of in vitro data is based on achievable serum concentrations of the drug, which may vary depending on dose, route of administration, degree of protein binding, site of infection, age and weight of the patient, and other factors. The MBC is defined as the lowest concentration of an antimicrobial agent needed to kill 99.9% of the initial organism inoculum.

The test was preformed by growing pure cultures of each of the test organisms in liquid culture. Turbidometric measurements were used to control the concentration of the culture. Serial dilutions of each test antibiotic were made in nutrient broth. The dilutions were calculated to cover the susceptible ranges for each organism for each agent. A standard amount of the test culture was added to each tube and the tube returned to an incubator (37±2° C.) for growth. The tubes were checked turbidometrically to determine bacterial growth. Below the MIC concentration the tubes showed an increase in optical density with time indicating bacterial growth. The lowest concentration of the antibiotic that showed no growth was the MIC. The “no growth” tubes were then subcultured in fresh medium. The “no growth” tube with the lowest concentration of antibiotic that showed no growth on subculturing was the MBC. Results are shown in Table 3.

B. Results:

TABLE 3
Antimicrobial (ppm)

| Organism | Tetracycline | Ofloxacin | Penicillin G | Cefaperazon | Erythromycin | Silver |
|---|---|---|---|---|---|---|
| S. pyogenes | 0.625/>5 | 1.25/2.5 | >5.0 | 0.313/1.25 | 0.003/0.019 | 2.5/5.0 |
| S. mutans | 0.625/>5 | 2.5/>5.0 | 0.521/>5 | 1.25/>5 | 0.009/0.019 | 2.5/10.0 |
| S gordonii | 0.156/0.625 | 2.5/5.0 | 0.009/0.039 | 1.25/1.25 | 0.005/0.019 | 2:5/10.0 |
| S. pneumoniae | 0.078/0.625 | 2.5/2.5 | 0.019/0.019 | 0.313/0.313 | 0.002/0.004 | 2.5/2.5 |
| S. faecalis | 0.313/>5 | 1.25/5.0 | 5.0/>5.0 | >5.0 | 0.009/1.25 | 10.0/10.0 |
| S. aureus | 0.313/>5 | 0.417/0.625 | 2.5/>5.0 | 5.0/5.0 | 0.039/>5.0 | 5.0/5.0 |
| P. aeruginosa | 0.078/5 | 0.156/0.313 | 0.13/>5.0 | 2.5/5.0 | 2.5/>5.0 | 1.67/5 |
| E. coli | 1.67/>5 | 0.104/0.156 | >5.0 | 0.625/>5.0 | 5.0/>5.0 | 2.5/2.5 |
| E. aerogenes | >5 | 0.078/0.156 | >5.0 | 2.92/>5.0 | >5.0 | 2.5/2.5 |
| E. cloacae | 1.67/>5 | 0.156/0.156 | >5.0 | >5.0 | >5.0 | 2.5/5.0 |
| S. typhimurium | 1.25/>5 | 0.078/0.156 | >5.0 | 1.25/2.5 | 5.0/>5.0 | 2.5/5.0 |
| S arizona | 0.625/>5 | 0.078/0.078 | >5.0 | 0.833/>5.0 | 4.17/>5.0 | 2.5/5.0 |
| S. boydii | 1.25/>5 | 0.078/0.156 | >5.0 | 0.625/0.625 | 5.0/>5.0 | 1.25/1.25 |
| K. pneumoniae | 2.5/>5 | 0.417/0.625 | >5.0 | >5.0 | >5.0 | 2.5/2.5 |
| K. oxytoca | 1.25/>5 | 10.104/0.156 | >5.0 | 1.25/>5.0 | >5.0 | 1.25/1.25 |

Data are presented as MIC/MBC (minimum inhibitory concentration/minimum bactericidal concentration) in parts per million (ppm)); “>” denotes that the concentration needed to obtain the MIC or the MBC was higher than test parameters measured for the test. For example, the highest concentration of tetracycline used on S. pyogene was 5 ppm. At that concentration there was still growth upon subculturing of the “no growth” tubes. Therefore, the MBC must be > (greater than) 5 ppm.

The MIC/MBC of E. coli strain O 157:H7, which has been associated with outbreaks of hemorrhagic diarrhea and colitis, was determined in a subsequent study. The MIC was determined to be 2.5 ppm and the MBC was determined to be 5 ppm.

C. Conclusion

The 10 ppm silver composition of the present invention was tested and found to be both bacteriostatic and bactericidal for all organisms tested. In other studies, this composition was compared to other commercially available colloidal silver products and found to have a superior activity to all other preparations tested (data not shown). The most interesting observation was the broad spectrum that the 10 ppm silver composition possesses. The antimicrobial activity that was observed was fairly constant independent of the particular organism tested. With the exception of Streptococcus faecalis and Streptococcus aureus (which had MIC values of 10 ppm and 5 ppm, respectively), MIC values ranged between 1.25 ppm and 2.5 ppm for both gram positive and gram negative organisms. The MBC values behaved similarly with values ranging from 1.25 ppm to 5 ppm with the exception of Streptococcus mutans, Streptococcus gordonii, and Streptococcus faecalis (which all had MBC values of 10 ppm). The data suggest that 10 ppm silver embodiment of this invention exhibits an equal or broader spectrum of activity than any one antibiotic tested. Antibiotics generally have restricted antibacterial spectra limited to susceptible organisms, but as the data demonstrate, the silver composition of the present invention is equally effective against both gram positive and gram negative organisms. The data suggest that with the low toxicity associated with silver, in general, and the broad spectrum of antimicrobial activity of this silver composition, this preparation can be effectively used as an alternative to antibiotics.

D. Reference for Preceding Example

1. U.S. EPA IRIS Report for Silver-CASRN 7440-22-4

2. Fox C L, Modak S M. Mechanism of Silver Sulphadiazine Action on Burn Wound. Infections. Antimicrobial Agents Chemother. 5:582-588. 1974.

3. Furchner, J E, Richmond C R, and G A Drake. Comparative Metabolism of Radionuclides in Mammals. IV. Retention of Silver-110m in the Mouse, Rat, Monkey, and Dog. Health Phys. 15:505-514.1968.

4. Grier, N. Silver and its Compounds in Disinfection, Sterilization, and Preservation. (Seymour S. Block, ed.) 2^{nd} Edn, pp 395-407. 1977.

5. Hindler, J A, and J H Jorgensen. Procedure in Antimicrobial Testing in Diagnostic Microbiology, (C R Mahon and G Manuselis, eds.) pp 63-91.1995.

Evidence of Efficacy of 32 ppm Silver Composition Against Pseudomonas Aeruginosa, Salmonella Chloraesuis and Staphylococcus Aureus

A. Methods

Pseudomonas aeruginosa ATTCC #15442, Salmonella choleraesuis ATTCC #10708 and Staphylococcus aureus ATCC #6538 were tested using the AOAC (Association of

Official Analytical Chemists AOC Methods, vol. 1, 15^{th} edition, 1990, AOAC Arlington, Va.) official methods 955.14, 95515 and 964.02. Nutrient broth (NBAOAC) tubes were inoculated from the stock culture, and the tubes incubated at 37±2° C. Transfers to fresh tubes of nutrient broth were made for three successive days with the final transfer being incubated at 37±2° C. for 48-54 hr. The Pseudomonas culture was decanted into a fresh tube to remove the pellicle.

The other cultures were vortexed for 3-4 seconds and allowed to stand for 10 min at room temperature. Finally the cultures were diluted 1:100 in peptone water (PEPW) to which equine serum was added to yield a 5% total organic challenge. Test carriers (10 mm long polished 304 stainless steel cylinders with an 8 mm outside diameter and 6 mm inside diameter) were soaked in challenge solution for 15 min, removed, drained and dried at 37±2° C. for 40±2 min prior to use.

Phenol Resistance. Five-one ml aliquots of each dilution of the test phenol were placed into sterile test tubes and allowed to equilibrate in a 20±2° C. water bath. At 30 second intervals, 0.5 ml of each challenge culture was added to the appropriate dilutions of phenol, agitated, and replaced into the water bath: After the appropriate exposure times of 5, 10, and 15 minutes, a loopful of suspension was removed from the assay tubes and transferred to tubes of letheen broth (LETH). The tubes of LETH were incubated at 37±2° C. for 2 days.

Carrier Titration. For titration of carriers, 10 ml blanks of peptone Tween® (brand of polysorbate) (PEPT) solution were prepared. Two carriers were placed into the individual tubes, representing the first 1:10 dilution. The tubes were agitated vigorously enough to get bacteria into solution and serial dilutions were made into 9 ml blanks of LETH medium. The dilution blanks were incubated at 37±2° C. The last tube with growth indicated the log, titer of organisms on the carrier. AOAC requires carriers to have minimum populations of 1×10⁴ cfu/carrier.

Test of Silver Composition. Using sterile glass pipettes, 10 ml aliquots of the prepared disinfectants were placed into sterile test tubes and allowed to equilibrate in a refrigerated water bath held at 20±2° C. Without touching the sides of the test tubes, one contaminated dried carrier was added at 30 second intervals to each tube of silver composition and placed back into the water bath. For each organism the disinfectant was tested against 60 dried contaminated carriers at 5 and 10 minute exposure intervals. Following exposure, the carriers were removed from the disinfectant and transferred to a tube of LETH. The culture tubes were incubated at 37±2° C. for 2 days and scored as positive (+) or negative (0) for growth of the challenge organism.

Controls. For each organism, a dried contaminated carrier was added to a tube of LETH as a positive control. Uninoculated media tubes served as negative controls. After incubation, all negative tubes were spiked with 1-100 colony forming units (cfu) of the corresponding organisms to demonstrate neutralization efficacy. To demonstrate growth promotion of the media, the negative control tubes were also inoculated with the same 1-100 cfu for all three organisms. The inoculating volumes were plated in triplicate onto soybean casein digest agar (SODA) to verify the inoculating titers. The tubes and plates were incubated at 37±2° C. until growth was seen in all tubes.

On the P. aeruginosa neutralization, the initial titer of inoculum was found to be >100 cfu which was too high for the protocol. Because all original tubes had been spiked, a simulated test was performed with same lot of media used in testing by placing carriers into disinfectant tubes from all three lots of silver compositions for 10 minutes. The carriers were sub-transferred to LETH blanks. These tubes were then spiked with 1-100 cfu of organism. The tubes were incubated as before and scored for growth or no growth. New tubes of sterile media from the same lot were also inoculated as a growth promotion verification.

B. Results

Initial testing using S. aureus demonstrated passing results for sample #1 and #2, but sample #3 failed. Upon investigation it was decided that sample #3 may have been damaged prior to shipment. A new bottle was obtained from the same lot as sample #3, and the new bottle was labeled as sample #4. The S. aureus challenge was repeated using sample #4. AOAC guidelines state that for any one time point and organism, only 1 carrier is allowed for growth for each lot tested.

Positive controls demonstrated growth and negative controls demonstrated no growth for all lots, time points, and organisms.

Carrier titration was run in duplicate for all organisms. The reported titer is an average of the replicates. For all three organisms, the average titer found on the carriers ranged from 5.5×10⁴ to 5.5×10⁶ cfu/carrier. AOAC requires carriers to have a minimum of 1.0×10⁴ cfu/carrier.

For P. aeruginosa 3/180 carriers showed growth at the 5 min test point and 2/180 carriers showed growth at the 10 min test point. For S. aureus 16/180 carriers showed growth at the 5 min test point and 2/180 carriers showed growth at the 10 min test point. For S. choleraesuis 6/180 carriers showed growth at the 5 min test point and 1/180 carriers showed growth at the 10 min test point.

The test Pseudomonas culture showed growth following a 5, 10 or 15 min treatment with 1:90 phenol and showed growth following a 5 or 10 min treatment with 1:80 phenol but no growth following 15 min treatment with 1:80 phenol. The Staphylococcus culture showed growth following a 5, 10 or 15 min treatment with 1:70 phenol and showed growth following 5 or 10 min treatment with 1:60 phenol but no growth following a 15 min treatment with 1:60 phenol. The Salmonella culture showed growth following a 5, 10 or 15 min treatment with 1:100 phenol but no growth following a 5, 10 or 15 min treatment with 1:90 phenol.

Evidence of Effectiveness of 32, 22, and 10 ppm Silver and 22 ppm Silver and 1.5% H₂O₂ and 10 ppm Silver and 10 ppm K₂S₂O₈ Against Salmonella and Escherichia Coli in Freshly Inoculated Beef Samples

A. Purpose of Example

The purpose of this example is to demonstrate the antimicrobial activity of the silver-based composition embodiments of the present invention on samples of beef flank steak inoculated on the exterior surface with a five strain cocktail of Salmonella species. or Escherichia coli O157:h7 at a high inoculum solution level (1×10⁶ cfu/cm²) and separately at a low inoculum solution level (1×10⁴ cfu/cm²) (cfu=colony forming unit).

B. Material and Methods

Beef Samples. Beef tissue samples were obtained from slaughter houses within 8 hours of evisceration. The rectus abdominus muscle was peeled off carcasses hanging in the chill cooler by making an incision between the 11^{th} and 12^{th} ribs and then peeling the muscle out along the natural seam. The aseptically retrieved samples were placed in plastic bags and on ice packs and were transported on the same day to the laboratory, where the samples were promptly packed in a Multi-Vac (A-300) and placed in a 4° C. cooler. Samples used for testing had a pH between 5.8 and 6.0 and were no more than 36 hours post evisceration. From randomly selected rectus abdominus muscles, 13×8 cm samples were cut and treated. After treatment, a 3.5 cm² flame sterilized stainless steel coring device and surgical scalpel were utilized to aseptically retrieve two meat cores per sampling interval from each sample. Tissue cores were placed in a sterile stomacher bag with 25 ml of 0.1% peptone and were mixed for two minutes in a stomacher (Lab Bender 400). Serial dilutions were prepared and spiral plated at 0 minutes, 20 minutes, 1 hour, 4 hours, and 24 hours post-treatment on selective and recovery media.

Bacterial Cultures. Bacterial cultures were obtained from the Kansas State University (KSU) stock culture collection and were stored using the “Protected Bead” storage system. The following cultures were used for the Salmonella specimen: S. lille (UGA), S. montevideo (UGA), S. typhimurium (UGA), S. agona (KSU 05 from CDC outbreak isolate), and S. newport (KSU 06 CDC outbreak isolate). The following cultures were used for the Escherichia coli specimen: E. coli O157:H7 (CDC 01,03), E. coli O157:H7 (USDA-FSIS 011-82 Rif resistant 100 ppm), E. coli O157:H7 (ATCC 43895 HUS associated Type I and II toxins Rif. Res.) and E. coli ATCC#23740 (Genotype K-12 prototrophic lambda).

Stock cultures were cultivated by placing one impregnated bead into a 5 ml solution of Difco® Tryptic Soy Broth (TSB) and incubating for 24 hours at 35° C. Next, a 0.05 ml loop of the respective culture was inoculated into a 5 ml solution of TSB and incubated for 24 hour at 35° C. to obtain a pure culture. After incubation, 1 ml of the respective culture was inoculated into 49 ml TSB and incubated for 24 hours at 35° C. Following incubation, samples were centrifuged (15,300×g at 4° C.), and the supernatant material decanted and the pellet was re-suspended with 50 ml of 0.1% peptone and centrifuged (15,300×g at 4° C.) a final time. The peptone was decanted and the remaining pellet was re-suspended with 10 ml of 0.1% peptone. The five 10 ml bottles of respective culture were mixed together to create a 50 ml cocktail containing 10⁹ cfu/ml of Salmonella species. The cocktail was diluted to 10⁶ cfu/ml or 10⁴ cfu/ml using 0.1% peptone. Cultures were confirmed by cultivation on selective and differential media, and biochemical analysis of presumptive colonies using API 20E kits.

Method of Inoculation. Samples of beef flank steak (rectus abdominus muscle) were trimmed to 13×8 cm (104 cm²) and were inoculated with a five strain cocktail of Salmonella species. or Escherichia coli O157:h7 at a high inoculum solution level (10⁶ log cfu/cm²) and separately at a low inoculum solution level (10⁴ log cfu/cm²). This inoculum was misted onto the tissue surface using a plastic spray bottle with samples contained within a sealed inoculum chamber. The actual Salmonella species. concentration on the meat surface was approximately 5.0 and 3.4 log cfu/cm² for the high and low level inoculum solution, respectively. For E. coli O157:H7, the respective meat surface inoculation levels were 4.2 and 3.9 log cfu/cm².

The beef samples were then hung vertically on stainless steel hooks attached to a motorized track that pulled the beef samples through a model spray cabinet (Kansas State University, Food Safety Laboratory) while spray treatments were applied. Treatments with either the silver compositions of this invention or deionized water were applied to the beef at 20 psi from a distance of 13 cm in the model pressure rinse cabinet for 20 seconds. The spray nozzle (BETE NF0580 303) delivered approximately 20 ml of solution to the surface of the beef sample. The temperature of solutions and treatment application room was approximately 14° C. After treatment, duplicate 3.5 cm² core samples were randomly drawn from the lateral surface of the beef sample at 0, 20, 60 and 240 minutes. Samples were cultivated and enumerated on selective differential and recovery media. Log reductions were calculated by subtracting the log₁₀ of cfu/cm² of the inoculated/treated samples at the specified sampling times (0, 20, 60, and 240 minutes) from the log₁₀ of cfu/cm² of the inoculated/untreated samples at 0 minutes. Sample treatment included the use of 32 ppm silver, 22 ppm silver, and 10 ppm silver compositions according to the present invention. Separately, combinations of 22 ppm Ag with 1.5 wght % hydrogen peroxide and 10 ppm Ag with 10 ppm peroxydisulfate (K₂S₂O₈) were tested.

C. Results with 32 ppm Silver Composition

The use of a composition of 32 ppm silver according to this invention produced a reduction in bacteria on beef steak. In the following, this reduction is expressed as the log₁₀ of the ratio of the number of bacteria in the control at time 0 to the amount of bacteria in the treated specimen at the sampling (i.e., treatment) time.

For Salmonella, at the lower initial bacteria level (10⁴), the following log reductions were recorded: 0.78 at 0 minutes, 1.11 at 20 minutes, 1.08 at 60 minutes, and 1.23 at 240 minutes. Thus, at 4 hours (240 minutes), the ratio of the initial bacteria count in the control to bacteria in the sample treated with 32 ppm silver is 10^{1.23}. For the higher initial bacteria level (10⁶), the following log reductions were recorded: 0.86 at 0 minutes, 0.95 at 20 min, 0.98 at 60 min and 1.17 at 240 min. The results indicate that the 32 ppm silver embodiment of this invention shows an effective bactericidal effect for Salmonella on beef steak. It will be appreciated that disinfecting a meat surface is an extreme challenge for any disinfectant.

For E. coli, for the lower initial bacteria level (10⁴), the following log reductions were recorded: 1.03 at 0 minutes, 1.28 at 20 minutes, 1.42 at 60 minutes, and 1.58 at 240 minutes. For the higher initial bacteria level (10⁶), the following log reductions were recorded: 0.65 at 0 minutes, 0.60 at 20 minutes, 0.83 at 60 minutes and 0.87 at 240 minutes. The results indicate that the 32 ppm silver embodiment of this invention shows an effective bactericidal effect for pathogenic E. coli on beef steak.

D. Results with 22 ppm Silver Composition

Results with Silver in Water. For Salmonella at the lower initial bacteria level (10⁴), the following log reductions were recorded: 0.41 at 0 minutes, 0.43 at 20 minutes, 0.48 at 60 minutes, and 0.68 at 240 minutes. For the higher initial bacteria level (10⁶), the following log reductions were recorded: 0.24 at 0 minutes, 0.24 at 20 minutes, 0.42 at 60 minutes and 0.61 at 240 minutes. The results indicate that the 22 ppm silver embodiment of this invention furnishes an effective bactericidal effect for Salmonella on beef steak.

Results with Silver in water and 1.5 wght % hydrogen peroxide. For Salmonella, for the lower initial bacteria level (10⁴), the following log reductions were recorded: 0.34 at 0 minutes, 0.33 at 20 minutes, 0.36 at 60 minutes, and 0.62 at 240 minutes. For the higher initial bacteria level (10⁶), the following log reductions were recorded: 0.28 at 0 minutes, 0.14 at 20 minutes, 0.30 at 60 minutes and 0.69 at 240 minutes. The results indicate that the 22 ppm silver with 1.5 wght % hydrogen peroxide embodiment of this invention provides an effective bactericidal effect for Salmonella on beef steak.

E. Results with 10 ppm Silver Composition

Results with Silver Composition in Water. For Salmonella, for the lower initial bacteria level (10⁴), the following log reductions were recorded: 0.38 at 0 minutes, 0.41 at 20 minutes, 0.39 at 60 minutes, and 0.61 at 240 minutes. For the higher initial bacteria level (10⁶), the following log reductions were recorded: 0.24 (at 0 minutes, 0.21 at 20 minutes, 0.41 at 60 minutes and 0.54 at 240 minutes. The results indicate that the 10 ppm silver embodiment of this invention provides an effective bactericidal effect for Salmonella on beef steak.

Results with Silver Composition in Water with 10 ppm K₂S₂O₈. For Salmonella, for the lower initial bacteria level (10⁴), the following log reductions were recorded: 0.26 at 0 minutes, 0.28 at 20 minutes, 0.35 at 60 minutes, and 0.58 at 240 minutes. For the higher initial bacteria level (10⁶), the following log reductions were recorded: 0.03 at 0 minutes, 0.16 at 20 minutes, 0.21 at 60 minutes and 0.36 at 240 minutes. The results indicate that the 10 ppm silver with 10 ppm potassium peroxydisulfate (K₂S₂O₈) embodiment of this invention provides an effective bactericidal effect for Salmonella on beef steak.

Evidence of Effectiveness of 10 ppm Silver for Treatment of Human Ailments

A. Purpose of Example

The purpose of this example is to demonstrate the utility of silver-based composition embodiments of the present invention for treating a variety of human ailments. The studies in this section were performed in Ghana, West Africa, at the Air Force Station Hospital under the direction of Dr. Kwabiah, at the Korie-Bu Teaching

Hospital under the direction of Sr. Sackey, and at the Justab Clinic/Maternity Hospital under the direction of Dr. Abraham. In total, fifty-eight (58) patients were treated using a silver/water composition of the present invention comprising 10 ppm silver. The composition was used both internally and externally as an alternative to traditional antibiotics. The ailments treated included malaria, upper respiratory tract infections, urinary tract infections, sinusitis, vaginal yeast infections, eye, nose and ear infections, cuts, fungal skin infections, and sexually transmitted diseases, such as gonorrhea.

B. Treatment Methods and Outcomes

Abdominal Pain and Diarrhea. The method comprises the step of administering approximately 5-25 ml of silver composition, one to five times a day orally until there was a response. One patient was treated with about 10 ml (about two teaspoons) of a composition of the present invention three times in one day. The patient had a full recovery in one day.

Bronchitis. The method comprises the step of administering ca. 2-25 ml of silver composition orally, one to five times a day until there was a response. Two patients were treated with about 5 ml (about one teaspoon) each of a composition of the present invention for two times a day for three days. The patients had a full recovery in three days.

Vaginal Yeast (Candida). The method comprises the step of administering ca. 5-25 ml of silver composition, one to five times a day as vaginal douches until there was a response. Five patients were treated with about 10 ml (about two teaspoons) each of a composition of the present invention for two times per day. The patients showed a full recovery within six days.

Conjunctivitis. The method comprises the step of administering ca. several drops of a silver composition, one to five times a day to the infected eye until there was a response. Two patients were treated with several drops of a composition of the present invention in each of the infected eyes for two times per day. The patients had a full recovery after one day.

External cuts and infection (including Staphylococcus skin infections, septic ulcers and infected abscesses). The method comprises the step of administering a silver composition, one to five times a day to the infected area until there was a response. Six patients were treated with about 5 ml (about one teaspoon) each of a composition of the present invention on the infected areas for two times per day. The patients showed a full recovery within three days.

External Otitis. The method comprises the step of administering a silver composition, one to five times a day to the infected ear until there was a response. Six patients were treated with approximately two drops of a composition of the present invention into the infected ears for three times per day. The patients showed a full recovery after about four days.

Otitis Media. The method comprises the step of administering a silver composition, one to five times a day to the infected ear until there was a response. One patient was treated with approximately two drops of a composition of the present invention comprising into the infected ear three times per day. The patient showed a full recovery in four days.

Fungal Skin Infection. The method comprises the step of administering a silver composition, one to five times a day topically to the infected area until there was a response. Two patients were treated with about ten ml (two teaspoons) each of a composition of the present invention three times per day. The patients showed a full recovery within eight days.

Gonorrhea. The method comprises the step of administering a silver composition to the infected area until there was a response. Two patients were each treated with about ten ml (two teaspoons) of a composition of the present invention three times per day. The patients showed an absence of symptoms within six days.

Malaria. The method comprises the step of administering a silver composition, one to five times a day orally to the patient until there was a response. Eleven patients were treated in a first study with about ten ml (two teaspoons) each of a composition of the present invention three times per day. The patients showed a resolution of symptoms within five days. More detailed Malaria protocols are discussed later herein.

Halitosis and Gingivitis. The method comprises the step of administering a silver composition, one to five times a day as a mouthwash until there was a response. Two patients were each treated with the composition as a mouthwash. There was a full resolution of symptoms within three days (gingivitis) and within one day (halitosis).

Pelvic Inflammatory Disease. The method comprises the step of administering about 5-25 ml of silver composition, one to five times a day as a vaginal douche until there was a response. One patient was treated with about 5 ml (approximately one teaspoon) of a composition of the present invention two times per day. The patient's symptoms resolved within five days.

Pharyngitis. The method comprises the step of administering a silver composition, one to five times a day as a gargle until there was a response. Four patients were each treated with about ten ml (two teaspoons) of a composition of the present invention three times per day. The patients showed full recovery within six days.

Retrovirus Infection (HIV). The method comprises the step of administering a silver composition, comprising 5 to 40 ppm silver one to five times a day orally area until there was a response. One patient exhibiting HIV (human immunodeficiency virus) was treated with about 5 ml (approximately one teaspoon) of a composition of the present invention two times per day. The patient's symptoms resolved within five days.

Sinusitis and Rhinitis. The method comprises the step of administering a silver composition, one to five times a day to the nose until there was a response. Six patients with nasal infections (four with sinusitis and two with rhinitis) were each treated with approximately two drops of a composition of the present invention comprising in their nasal passages three times per day. The patients showed full recovery within four days.

Tonsillitis. The method comprises the step of administering a silver composition, one to five times a day as a gargle until there was a response. One patient was treated with a composition of the present invention three times per day. The patient showed full recovery within seven days.

Upper Respiratory Tract Infection. The method comprises the step of administering a silver composition, one to five times a day orally until there was a response. Two patients were each treated with about 5 ml (approximately one teaspoon) of a composition of the present invention three times per day. The patients showed full recovery within six days.

Urinary Tract Infections. The method comprises the step of administering a silver composition, one to five times a day orally until there was a response. Three patients were each treated with about ten ml (two teaspoons) of a composition of the present invention two to three times per day. The patients showed full recovery within six days.

C. Discussion

These results are consistent with the various in vitro tests reported herein. Essentially, the silver composition is extremely effective against a large number of microbes in vitro. However, the tests indicate that this effectiveness remains even in the presence of a large amount of organic material. The silver compositions are widely effective in vivo where the organic background is extremely high. Many other disinfecting agents are ineffective in the presence of a large amount of organic material and/or are too caustic or toxic to be used in vivo.

Additional Malaria Study in Ghana, Africa

Another more regimented study was also conducted in Ghana. The purpose of this study was to utilize a very specific protocol and to focus only on the curative properties of the 10 ppm silver/water composition of the present invention on patients who had contracted malaria.

The purpose of this protocol was to devise a procedure whereby the 10 ppm silver/water solution produced according to the methods herein could be tested for its' possible curative properties in treating patients who have contracted a Malaria infection with any of the four (4) Plasmodium species. An overview of the protocol is as follows:

The trials were carried out in medical clinics or in hospitals by medical doctors (MD's) who were very familiar with the disease and its health ramifications. There were a total of 16 patients examined per doctor, and the patients were required to take the silver product twice a day for five days, as well as have their blood drawn one day before the trial began, and then every day until the blood test showed that the parasite had been eliminated for at least two days. The patients would only be paid if they adhered completely to the schedule for taking the silver and for obtaining the daily blood tests.

Details of Protocol:

Number of Medical Doctors (MDs) to be involved in testing: 2

Number of patients to be tested per physician: 16—8 males and 8 females

Total number of days for the trial: 15

Dose of 10 ppm silver/water composition to be given for treatment of patients: A total daily dose of one ounce divided into two equal doses; one-half ounce (3 tsp) taken in the morning and one-half ounce (3 tsp) taken in the evening. The patients were treated with the Silver/Water solution for the first five (5) days of the total 15 day trial, or if the parasite was not completely gone by day five, silver/water treatment was continued until the parasite was gone, or until day 15, which ever occurred first.

In the event of a patient whose parasites were gone by day two or three, the silver/water was continued until day five, and a note was made in the records as when the parasite was completely gone.

In the event of a patient who was still harboring parasite after having taken the silver/water for 15 days, the trial would be terminated as usual, and this patient recorded as a failure to cure in the records.

For the patient who was cured in less than five days, the date of complete parasite disappearance was recorded, the patient continued to receive silver/water until day five, and continued in the trial to day 15.

Blood Tests:

Blood tests to be used: The presence (or absence) of parasites in the patients blood was determined by either the Acridine Orange stain test, or the Giemsa stain test, on thin or thick blood smears from each of the patients. The patients blood was tested on day zero (0) to ascertain that they did, in fact, have an active case of malaria. If the blood test confirmed an active case of malaria, then that patient was screened for acceptance into the trial. Screening included recording of vital data such as name, age, patient reported onset of disease, informing the patient of what was required of them during the trial, what they would be paid for full compliance, and the fact that failure of compliance would result in being dropped from the trial with no remuneration. For patients that agreed to join the trial, they were given a written set of instructions telling them how to take the silver product each day, where to go each day for their blood tests, and emphasizing the necessity of complete adherence to the protocol of the trial in order to be paid for any of the days.

The above protocol was strictly adhered to in the most recent study in Ghana, Africa. All patients received the same dosage, and their blood was checked daily to determine the existence of the plasmodia parasites. The following Table 4 lists portions of the earlier studies discussed above (Study 1 and Study 2), as well as new Study 3, which followed the protocol set forth immediately above herein.

Summary

TABLE 4

| Study | 1 | 2 | 3 | 4 |
|---|---|---|---|---|
| Number of Patients | 11 | 16 | 16 | 13 |
| Age Range | 8-75 | 2-90 | 3-61 | 15-57 |
| Males/Females | NA | NA | 8/8 | 6/7 |
| Average daily dosage | 10 ml | 5 ml | 15 ml | 15 ml |
| Shortest recovery time+ | 3 days | 3 days | 2 days | 3 days |
| Longest recovery time | 7 days | 10 days | 8 days | 6 days |
| Average recovery time | 5.0 days | 6.3 days | 4.3 days | 4.0 days |
| # patients checked for | 0 | 7 | 16 | 13 |
| plasmodia | | | | |
| # patients w. plasmodia | 0 | 0 | 0* | 0* |
| # treatment failures | 0 | 0 | 0 | 0 |

+Recovery times were those taken by the patients to be asymptomatic, as estimated by the doctors.
*Each of these patients was checked daily for 14 days. After six days, each of their blood samples tested negative for plasmodia.

Clearly the 10 ppm silver/water solution of the present invention had significant positive effects against the malaria parasite.

Evidence of Efficacy 100 ppm Silver Against Malaria (In Vitro)

Introduction

On a global scale, malaria has been and remains a major public health concern. The disease is caused by parasitic protozoa of the genus Plasmodium. The life cycle of this organism is complex, with the parasite alternating between sexual reproduction in an invertebrate (mosquito) host and asexual reproduction in a vertebrate host In addition to mammals as vertebrate hosts, birds and reptiles also serve as hosts for malarial parasites. The portion of the life cycle in the mosquito is the sporogonic phase, leading to formation of sporozoites which are injected by the vector into the vertebrate host at time of feeding. Sporozoites give rise to the schizogonic phase, with proliferation of the parasites in erythrocytic and exoerythrocytic sites. The parasite is extracellular during its sporogonic phase, shifting to an intracellular location during the schizogonic stages of development. In vitro cultivation of the parasite requires simulating conditions in the mosquito vector for the sporogonic phase of the life cycle and, for the schizogonic phase, conditions promoting growth in exoerythrocytic and erythrocytic locations of the vertebrate hosts.

Malaria is one of the world's most prevalent parasitic diseases and ranks no less than third in the world among major infectious disease in terms of mortality. The protozoal parasite that causes malaria is from Plasmodium genus. Four species of Plasmodium protozoa cause malaria: Plasmodium falciparum, Plasmodium vivax, Plasmodium malariae, and Plasmodium ovale. Transmitted principally by the Anopheles mosquito, malaria infections may also occur from contacting infected blood, such as from blood transfusions.

Classic symptoms of malaria include fever, headaches, chills, vomiting, shivering and convulsions. In some rare forms of falciparum malaria, chills and fever may be absent and the patient may present with delirium or coma. Remission periods can last from a few weeks to several months. Severe anemia is often attributed to the cause of death from malaria infection.

Plasmodium falciparum:

This parasite has several important features. These include the crescent shape of the gametocyte, the slow rate of growth of the latter and the localization of the pigment around the nucleus (perinuclear distribution) which is absent in the gametocytes of other primate malarial parasites.

P. falciparum also differs from the other human species in its greater virulence and lethal effects, while schizogony of the erythrocytic stages is largely confined to the capillaries and sinusoids of the internal organ. The popular name for the disease caused by P. falciparum is “malignant tertian malaria”.

### Materials and Methods

### Citrated Saline:

| | | |
|---|---|---|
| Sodium Chloride | 9 | gm |
| Sodium Citrate | 20 | gm |
| Distilled Water | 1000 | ml. |

### Giemsa Stain:

| | |
|---|---|
| Giemsa Satin Powder | 75 gm |
| Absolute alcohol | 75 ml, |
| Glycerol | 25 ml. |

### Field's Stain:

Field's Solution #1

1. Dissolve 1.6 g of methylene blue in 1 liter of distilled water.

2. Dissolve 2.6 g of Na₂HPO₄ (anhydrous) to the solution from step 1.

3. Dissolve 1 g of Azure 1 in the solution from step 2.

4. Dissolve 2.6 g of KH₂PO₄ in the solution from step 3.

5. Place on mild heat with stirring or shaking for 45 minutes to 1 hour.

6. Let stand at room temperature for 24 hours.

7. Filter.

Field's Solution #2

1. Dissolve 2 g of Eosin Y in 1 liter of distilled water.

2. Dissolve 2.6 g of Na₂HPO₄ in the solution from step 1.

3. Dissolve 2.6 g of KH₂PO₄ in the solution from step 2.

4. Filter.

Wright's Stain:

| | |
|---|---|
| Wright's stain powder | 6.0 g |
| Giemsa stain powder | 0.6 g |
| Methanol | 1,000 ml |

Stir overnight and filter before use.

### Human Serum/Plasma of Blood Group AB+

Human Serum/Plasma of blood group A+
RPMI—1640 Incomplete Medium
(Personal Communication Dr. Sutar, Haffkine Institute, Parel)
RPMI—1640 Complete Medium
(Personal Communication Dr. Sutar, Haffkine Institute, Parel)

### Collection and Processing of Infected Blood

Parasitised erythrocytes were obtained by collecting 6 ml aliquots of blood in 1 ml citrated saline by venipuncture from clinically diagnosed cases of Plasmodium vivax and Plasmodium falciparum malaria from Kasturba Infectious Disease Hospital, Bombay. The blood samples were collected in 10 ml sterile vials. The samples were examined by preparing thin smears and staining the smears with 10% Giemsa's Stain/Field's Stain/Wright's Stain, for identification and confirmation of spp. of malarial parasite. The level of percent of parasitemia of the sample was recorded.

The parasitised blood cells were washed twice with incomplete medium and once with complete medium and 6% cell suspension was prepared in complete medium. Cultures were set up by dispensing 0.5 ml of suspension in each petri dish. To this 1.5 ml complete medium was added and plates were incubated in an atmosphere of 5% CO₂ and 14-17% O₂ The medium was changed daily by aspirating the old medium with a sterile pasture pipette and adding 1.5 ml complete medium. Cultures were maintained by adding fresh cells (from blood group A+ or AB+; washed and cell suspension prepared in the same way) after one week, with washing 2 times/week, until the target parasitic index reached to ≧1%. If the initial parasitic index was more than 1%, then blood medium mixture (BMM) was used directly for drug sensitivity (Thanh, 2001) and (Tasanor, 2002)

### Preparation of Smears and Staining Procedure

Cultures were washed twice per week. For washing, the cultures were removed from plates and transferred to centrifuge tubes. About 5 ml of incomplete medium was added to each centrifuge tube and mixed thoroughly. The tubes were centrifuged at about 1000-1500 rpm for about 10 minutes. After about 10 minutes, the tubes were removed from centrifuge and the supernatant fluid was discarded. Later, the cultures were subjected to two more similar washes, one with incomplete RPMI-1640 and another with complete RPMI-1640 medium. After three washes the cultures were transferred to separate Petri dishes. A smear was prepared from each culture and stained with Giemsa/Field's/Wright's stain. About 1.5 ml of medium was added to each plate and the smears were examined under an optic microscope for parasitic index or % parasitemia for each culture was recorded. Fresh erythrocytes were added to each plate every week (Pradhan, 1984)

Preparation of Smear

A drop of culture from the plate was taken on a micro slide. A thin smear was made and air dried. This smear was fixed by dipping the slide in a coupling jar containing absolute alcohol. A 10% Giemsa stain solution was prepared and used for staining the smears. The slides were kept immersed in a 10% Giemsa stain solution for about 30-40 minutes and then washed under tap water.

Parasitic Index

Parasitic index was calculated by counting the number of parasites per 100 erythrocytes in thin blood smears. Minimum 100 fields or 10,000 RBCs were observed for this purpose.

Culture Systems

Plate cultures were prepared with a 5% hematocrit and about 1% parasitemia. The lower the initial parasitemia is, the greater the increase in numbers of parasites that will occur during in-vitro growth.

Drug Sensitivity

16 mm flat bottom sterile Microwell plates were used for drug sensitivity testing. One was used for one sample. First two wells were used for control and received 50 μl patients BMM or culture and 50 μl RPMI complete medium and no drug. For test 50 μl of culture or patients BMM was mixed into well containing 50 μl of engineered silver nanoparticles (ESNP) with various concentrations. Microwell plates were covered and incubated at about 37° C. in a candle jar for about 48 hrs. Most of the parasites enter in schizont stage at the end of 48 hours of incubation. After incubation using micropipette the supernatant medium was removed; blood from each well was taken to prepare smears and observed for the schizont development. The test has been evaluated by counting the number of parasites in stained pre-incubation and post-incubation films and ESNP related inhibition of schizont formation. For a valid test, the control well should show ≧10% schizont maturation. (Wernsdorfer and Wernsdorfer, 1995).

### Results

| Species | % Decrease of Parasites |
|---|---|
| Plasmodium falciparum | 94% |
| Plasmodium vivax | 92% |
| Plasmodium berghei | 90% |

The in-vitro test used as an indication of antimalarial efficacy shows conclusively that ESNP—100 ppm is able to reduce the parasitic count in-vitro. This is of significance since the parasites collected were from patients showing rigors with elevated temperatures and classical symptoms of malarial infections.

Evidence of Efficacy of 10 ppm Silver Against Tuberculosis Bacteria

A. Purpose

The purpose of this example is to demonstrate the efficacy of a silver composition of the present invention against the bacteria that cause tuberculosis. This example describes the procedures for evaluation of the present invention for tuberculocidal efficacy. The methodology is based on the Tuberculocidal Activity Test Method as accepted by the EPA on Dec. 11, 1985. [Refer to United States Environmental Protection Agency, 1986. Office of Pesticides and Toxic Substances. Data Call-In Notice for Tubercuolocidal Effectiveness Data for All Antimicrobial Pesticides with Tuberculocidal Claims. (Received Jun. 13, 1986).

B. Material and Methods

Materials. The silver composition of the present invention comprised 10 ppm silver in water. The silver composition was evaluated employing a liquid to liquid matrix against Mycobacterium bovis BCG (TMC 1028). This organism causes tuberculosis in animals and can cause tuberculosis in humans. It is used as a “stand-in” for M. tuberculosis, the major cause of human tuberculosis, as tests have shown it to have a similar susceptibility to M. tuberculosis. The test organism was exposed to the silver composition in duplicate at four exposure times and quantified using membrane filtration.

Procedure. A vial of frozen stock culture was removed from storage and thawed. An equal volume of buffered gelatin (BUGE) was added to the cell suspension and homogenized with a Teflon° (brand of polytetrafluoroethylene) tissue grinder for 1 minute while keeping the culture at 0 to 4° C. in an ice bath. The homogenized cell suspension was diluted with saline Tween® 80 (brand of polysorbate) solution (ST80) to approximately 10⁷ cfu/ml.

Challenge Titration. Tenfold serial dilutions of the culture were prepared in dilution blanks containing 9 ml of neutralizer broth (NEUB) through a 10⁻⁶ dilution. Three 1 ml aliquots of the appropriate dilutions were membrane filtered by first adding 10-20 ml physiological saline solution (PHSS) to the filter housing and then adding a 1 ml aliquot of the appropriate dilution. The filter was then rinsed with approximately 100 ml of PHSS. The filters were aseptically removed from the filter housing and placed onto 7H11 agar plates. The plates were incubated in a humidified chamber at 37±2° C. for 21 days.

Positive Control. A tube containing 9 ml of ST80 was prepared and equilibrated to 20±0.5° C. At time 0, 1 ml of test organism culture was added to the tube (1:10 dilution). The sample was held for 60 minutes. Tenfold serial dilutions were prepared in dilution blanks containing 9 ml of NEUB through 10⁻⁶ dilution. Three 1 ml aliquots of the appropriate dilutions were membrane filtered by first adding 10-20 ml PHSS to the filter housing and then adding a 1 ml aliquot of the appropriate dilution. The filter was rinsed with approximately 100 ml PHSS. The filters were aseptically removed from the filter housing and placed onto 7H11 agar plates. The plates were incubated in a humidified chamber at 37±2° C. for 21 days.

Tests. Two 25×150 mm tubes containing 9 ml of the test sample were equilibrated to 20±0.5° C. in a water bath. To each tube containing the test disinfectant (i.e., silver composition), 1 ml of test organism culture was added. The tube was mixed by swirling and placed back into the water bath. At 15, 30, 45, and 60 minutes, 1.0 ml aliquots of the disinfectant-cell suspension were transferred to 9 ml of NEUB and mixed thoroughly. Tenfold serial dilutions were prepared in dilution blanks containing 9 ml of NEUB through the 10⁻⁶ dilution. Three 1 ml aliquots of the appropriate dilutions were membrane-filtered by first adding 10-20 ml PHSS to the filter housing and then adding a 1 ml aliquot of the appropriate dilution. The filter was rinsed with approximately 100 ml PHSS. The filters were aseptically removed from the filter housing and placed onto 7HII agar plates. The plates were incubated in a humidification chamber at 37±2° C. for 21 days.

Phenol Control. To demonstrate minimum culture viability and resistance, the culture was tested against a 0.8% phenol solution. A 1 ml aliquot of test organism culture was placed into 9 ml of the phenol solution equilibrated to 25±0.5° C. and incubated for 20 minutes. After the exposure period, 1 ml from the phenol/organism solution was removed and added to 9 ml of NEUB. Tenfold serial dilutions were prepared in dilution blanks containing 9 ml of NEUB through 10⁻⁶ dilution. Three 1 ml aliquots of the appropriate dilutions were membrane filtered by first adding 10-20 ml PHSS to the filter housing and then adding a 1 ml aliquot of the appropriate dilution. The filter was rinsed with approximately 100 ml PHSS. The filters were aseptically removed from the filter housing and placed onto 7H11 agar plates. The plates were incubated in a humidified chamber at 37±2° C. for 21 days.

Neutralization verification. A 1 ml aliquot of the disinfectant was added to 8 ml of NEUB. The disinfectant/neutralizer broth was allowed to equilibrate to the same temperature as the test samples. One ml of test organism culture was added to the mixture and mixed thoroughly. Incubation was continued for the approximate time it would take to filter a sample. Additionally, a 1 ml aliquot of test organism was added to 9 ml of NEUB and mixed thoroughly (1:10 dilution). Tenfold serial dilutions of both tubes were prepared in dilution blanks containing 9 ml of NEUB thought 10⁻⁶ dilution. Three 1 ml aliquots of the appropriate dilutions were membrane filtered by first adding 10-20 ml PHSS to the filter housing and then adding a 1 ml aliquot of the appropriate dilution. The filter was rinsed with approximately 100 ml PHSS. The filters were aseptically removed from the filter housing and placed on 7H11 agar plates. The plates were incubated in a humidified chamber at 37±2° C. for 21 days.

C. Results

The starting titer for the challenge culture was 4.7×10⁷ cfu/ml. The positive control titer was 6.5×10⁶ cfu/ml. The media used in this study effectively demonstrated neutralization with a 95.2% recovery in a disinfectant/neutralizer solution when compared to a media blank.

For the test plates, expected counts were underestimated and therefore the reported counts exhibit “>” to mark that the count is an estimation and that accurate counts are beyond the limit of detection for the dilutions plated.

In calculating the log and percent reductions of the disinfectant against M. bovis, the estimated counts which have “greater than” counts resulted in “less than” log and percent reductions (“<”). The purpose of this is to demonstrate that the results are an estimation and beyond the accurate limit of detection for the dilutions plated. All reductions were calculated using the positive control as the initial starting titer of the organism. The results for log and percent reductions are summarized below. As a measure of the resistance of the challenge culture, the phenol resistance of the M. bovis showed a ˜1.81 log reduction with 20 minutes of exposure to 0.8% phenol.

Replicate One:

| Exposure time | Log reduction | Percent reduction |
|---|---|---|
| 15 minutes | <0.12 | <12.3% |
| 30 minutes | <0.22 | <40.0% |
| 45 minutes | <1.57 | <97.2% |
| 60 minutes | <1.56 | <97.2% |

Replicate Two:

| Exposure time | Log reduction | Percent reduction |
|---|---|---|
| 15 minutes | <0.26 | <44.8% |
| 30 minutes | <0.20 | <36.9% |
| 45 minutes | <1.58 | <97.3% |
| 60 minutes | <1.53 | <97.1% |

D. Conclusions

The use of silver compositions of the present invention is effective against tuberculosis bacteria. A method comprising the step of administering silver compositions of the present invention is effective against tuberculosis organisms.

Evidence of Efficacy of 10 ppm Silver Against Candida algicans ATCC #10231, Trichomonas vaginalis ATCC #20235, and MRSA Staphyloccocous aureus ATCC #BAA-44

A. Purpose of Example

The purpose of this example is to illustrate the efficacy of silver compositions of the present invention against Candida albicans ATCC10231, Trichomonas vaginalis ATCC 20235, and drug resistant Staphylococcus aureus ATCC BAA-44.

Candida albicans, a yeast, and Trichomonas vaginalisis, a protozoa, can cause numerous health problems including vaginal infections, diaper rash, and thrush. The results below show that silver compositions of the present invention produced nearly a 100% kill of both organisms. The results show the utility of silver compositions of the present invention in a feminine hygiene product and in a diaper rash product.

Staphylococcus aureus can cause serious blood poisoning when it enters a wound. It once was easily treated with penicillin, but the organism has now mutated to the point where it is totally resistant to penicillin. The next defense on the antibiotic ladder has been methicillin, but methicillin-resistant strains have become increasingly common, especially in hospitals. These strains are known as MRSA (methicillin-resistant Staphylococcus aureus) and have been dubbed the “superbug.” People who contract

MRSA can die in a matter of days. In the results reported in this example, a silver composition of the present invention was found to kill 91.6% of the MRSA in just 10 minutes, and 99.5% in an hour. The results show the utility of silver compositions of the present invention in killing MRSA, a known infectious threat.

B. Methods and Results

Employing the USP Preservative Rapid Challenge Test with a composition of the present invention comprising 10 ppm silver in water, the following results were obtained. These results show that silver compositions of the present invention can be effective against yeast infections, protozoa infections, and drug resistant bacteria infections.

Candida albicans ATCC #10231. The initial concentration of Candida albicans yeast was 6.8×10⁵ cfu/ml. After contact for either 10 minutes, 30 minutes, 1 hour, or one day with the silver composition, there were no colonies detected.

Trichomonas vaginalis ATCC #30235. The initial concentration of Trichomonas vaginalis protozoa was 6.0×10⁴ cfu/ml. After contact with the silver composition for either 10 minutes, 30 minutes, 1 hour, or one day, there was 0% motility of 100 Organisms. That is, one hundred (100) Trichomonas vaginalis parasites were analyzed via microscopy for motility of flagella. None of the one-hundred (100) parasites demonstrated motility after only ten (10) minutes of contact with the silver composition indicating inhibitory or lethal properties of the silver composition on the parasites. The outer membranes of twenty-five (25) percent of the parasites had ruptured after contact of one (1) day.

Staphylococcus aureus MRSA ATCC #BAA-44. The initial concentration of methicillin-resistant Staphylococcus aureus (MRSA) was 6.0×10⁶ cfu/ml. After contact with the silver composition, there were 500,000 cfu/ml detected after 10 minutes contact (91.6% killed), 70,000 cfu/ml after 30 minutes contact (98.8% killed), 30,000 cfu/ml after 1 hour contact (99.5% killed), and fewer than 10 cfu/ml after one day contact (virtually total kill).

Evidence of the Efficacy and Lack of Cytotoxicity of 10 ppm Silver, 14 ppm Silver +1.5% H₂O₂, and 22 ppm Silver in Inhibiting DNA Polymers and Reverse Transcriptase in the Context of Hepatitis B

A. Purpose of Example

The purpose of the example is to illustrate the efficacy of silver compositions of the present invention against hepatitis B. This example shows that silver compositions of the present invention have antiviral properties. Any agent used in antiviral therapy should exhibit little or no cytotoxicity so cytotoxicity of the silver compositions was analyzed.

Hepatitis B is caused by a DNA virus of the hepadnaviridae family of viruses. The Hepatitis B Virus (HBV) is a 3.2 kb DNA virus, replicating almost exclusively in the liver cells (hepatocytes). Replication involves two main enzymes: DNA polymerase and reverse transcriptase. The results of this example show that silver compositions of the present invention interfere with replication involving either DNA polymerase or reverse transcriptase. The results of this example show that silver compositions of the present invention have antiviral properties. The results of this example show that silver compositions of the present invention can be effective against hepatitis B.

As further detail, when hepatitis B enters the body of a new host, it infects the liver if it gets past the host's immune system. In the infection, the virus attaches to the membrane of a liver cell, and the core particle of the virus enters the liver cell. The core particle then releases its contents of DNA and DNA polymerase into the liver cell nucleus. Within the liver cell, the virus replicates via reverse transcription and translation processes, which involve reverse transcriptase and DNA polymerase enzymes. The DNA polymerase causes the liver cell to make copies of hepatitis B DNA. These copies of the virus are released from the liver cell membrane into the blood stream. From there, they can infect other liver cells and thus replicate effectively. The incubation period of the hepatitis B virus is about 6 to 25 weeks (i.e., time before physical and generally detectable histological or physical symptoms occur). However, there are several biochemical and histological changes that occur in the early stages following infection with the hepatitis B virus.

B. Materials

Solutions comprising 10 ppm, 14 ppm, 22 ppm, and 32 ppm silver compositions according to the present disclosure were used. The nucleotides dATP, dGTP, dCTP, and [³H]-dTTP were obtained from standard commercial sources, as were the compounds lamivudine (a synthetic antiretroviral agent) and zidovudine (AZT). Isolated Hepatitis B virus was freshly obtained from a person suffering from Hepatitis B infection and was taken up by Haffine Institute, Mumbai INDIA (a WHO certified testing laboratory). Test cell cultures (Vero and Hep2) were grown as confluent monolayers by typical cell culture methods.

C. Methods

### 1) Procedure for Test of DNA Polymerase Inhibition

Overall approach. Hepatitis B viral extracts from human subjects are incubated with radiolabelled nucleotides and an active inhibitor. Percent inhibition is calculated based on the amount of de novo viral nucleic acid synthesized with respect to lamivudine as a positive control and phosphate buffer saline (PBS) as a negative control.

Specific procedure. Isolated Hepatitis B virus was lysed to extract free polymerase enzyme, which is free from contaminating enzymes. A virus extract (25 ml) was added to a reaction mixture comprising dATP, dGTP, dCTP and [³H]dTTP nucleotides (25 ml). Active inhibitor (3 ml) was added to the mixture comprising virus extract and nucleotides. The resultant mixture was incubated at 37° C. for 24 hours.

A separate negative control experiment was performed in which phosphate buffer saline (PBS, 3 ml) was used instead of the inhibitor (3 ml).

A separate positive control experiment was performed in which a known DNA polymerase inhibitor (3 ml of lamivudine at a concentration 3 mg/ml) was used instead of the tested inhibitor (3 ml).

The reaction was stopped by adding 25 ml EDTA and 25 ml TCA (trichloroacetic acid). The reaction mixture was then spotted on ionic paper (DEAE paper). The paper was washed three times with TCA and then with ethyl alcohol. The filter paper was air dried and put into a scintillation vial with a scintillation cocktail. Radioactivity was measured by a liquid scintillation counter (Blue Star). As a counting control, a blank silver composition was run through the complete procedure without viral load, to check any potential interference in the scintillation counter method.

A reference for this method is P. S. Venkateswaran, I. Millman, and B. S. Blumberg, “Effect of an extract from Phyllanthus niruri on hepatitis B and woodchuck hepatitis viruses: in vitro and in vivo studies,” Proc. Natl. Acad. Sci., USA, 1987, 84, 274-278, which is incorporated herein by reference.

### 2) Procedure for Test of Reverse Transcriptase Inhibition

A commercial viral enzyme preparation of Moloney murine leukemia virus reverse transcriptase (MoMuLV) having Poly(A)dT (primer for RT) was used. 50 ml of the MoMuLV preparation was combined with a mixture of dATP, dGTP, dCTP and [³H]dTTP nucleotides.

This mixture was combined with 3 ml of the inhibitor to be tested, and the resultant mixture was incubated at 37° C. for 24 hours.

A negative control experiment was performed in which phosphate buffer saline (PBS, 3 ml) was used instead of the inhibitor.

A positive control experiment was performed in which a known reverse transcriptase inhibitor (3 ml of AZT at a concentration 0.625 microgram/ml) was used instead of the tested inhibitor.

The reaction was stopped by adding 25 ml EDTA and 25 ml TCA. The reaction mixture was then spotted on ionic paper (DEAE paper). The paper was washed three times with TCA and then with ethyl alcohol. The filter paper was air dried and put in a scintillation vial with a scintillation cocktail. Radioactivity was measured by a liquid scintillation counter (Blue Star).

### 3) Procedure for Testing Cytotoxicity

Cells were prepared from healthy, confluent Vero and Hep2 cell cultures that were maintained by passage every 3-4 days. One day prior to the test cells were released from the cultures using standard techniques and suspended in a growth medium and dispensed into wells of a microtiter plate and placed in a 5% CO₂ incubator at 37±2° C. An aliquot (100 ml) of each test substance was introduced into a well (in triplicate) with 100 ml of PBS as a control. Every 24 hrs the wells were examined under high power of an inverted microscope to check for any cytopathic effect (CPE). All results are shown in the following Table 5.

D. Results

Results for test of reverse transcriptase inhibition:

TABLE 5a

| Sample | % Inhibition |
|---|---|
| negative control (PBS) | 0 |
| positive control (AZT) | 31.33 |
| Silver, 10 ppm | 89.52 |
| Silver, 14 ppm and 1.5% H2O2 | 86.93 |
| Silver, 22 ppm | 84.46 |

Results for test of DNA polymerase inhibition:

TABLE 5b

| Sample | % Inhibition |
|---|---|
| negative control (PBS) | 0 |
| positive control (lamivudine) | 31.33 |
| Silver, 10 ppm | 77.73 |
| Silver, 14 ppm with 1.5% H2O2 | 65.6 |
| Silver, 22 ppm | 60.89 |

Silver compositions of the present invention are highly effective at inhibiting DNA polymerase

Results for test of reverse transcriptase inhibition:

TABLE 5c

| Sample | % Inhibition |
|---|---|
| negative control (PBS) | 0 |
| positive control (AZT) | 18.06 |
| Silver, 10 ppm | 89.52 |
| Silver, 14 ppm with 1.5% H2O2 | 86.93 |
| Silver, 22 ppm | 84.46 |

Thus, silver compositions of the present invention inhibit reverse transcriptase. Silver compositions of the present invention would be expected to be effective against human ailments propagated by viruses, such as hepatitis B.

Results for test of cytotoxicity:

TABLE 5d

| Sample | Vero | Hep2 |
|---|---|---|
| control (PBS) | No CPE | No CPE |
| Silver, 10 ppm | No CPE | No CPE |
| Silver, 14 ppm with 1.5% H2O2 | CPE positive | CPE positive |
| Silver, 22 ppm | No CPE | No CPE |

These results indicate that the silver composition is essentially non-cytotoxic. As expected, hydrogen peroxide, which is known to be cytotoxic, shows a cytotoxic effect. Thus, the silver should be harmless to cells when used in vivo.

12. Evidence of Efficacy of Silver Composition as Water Disinfectant

A. Purpose

Tests were carried out to demonstrate the efficacy of the inventive composition in disinfecting drinking water.

B. Methods

A sample of raw river water was spiked with two loopfuls of Klebsiella oxtyoca. 100 ml aliquots of this of this spiked water solution were brought to 0.05 ppm, 0.1 ppm, 0.2 ppm, 0.5 ppm, or 1.0 ppm of inventive silver composition. After an incubation of 5-60 minutes, the samples were membrane filtered. The filter was rinsed with approximately 100 ml sterile water. The filters were aseptically removed from the filter housing and placed on coliform nutrient agar plates. The plates were incubated under growth conditions for 24 hours and counted.

TABLE 6

| | | | Total Coliform | |
|---|---|---|---|---|
| Sample | Silver (ppm) | Contact (min) | (per ml) | Cfu/100 ml |
| raw water | — | — | 36 | TNTC |
| 1 | 1.00 | 5.0 | 0 | 0 |
| 2 | 1.00 | 10.0 | 0 | 0 |
| 3 | 1.00 | 15.0 | 0 | 0 |
| 4 | 1.00 | 30.0 | 0 | 0 |
| 5 | 0.50 | 10.0 | 0 | 0 |
| 6 | 0.50 | 30.0 | 0 | 0 |
| 7 | 0.50 | 60.0 | 0 | 0 |
| 8 | 0.20 | 5.00 | 0 | 0 |
| 9 | 0.20 | 10.0 | 0 | 0 |
| 10 | 0.20 | 30.0 | 0 | 0 |
| 11 | 0.20 | 60.0 | 0 | 0 |
| 12 | 0.10 | 10.0 | 0 | 0 |
| 13 | 0.05 | 20.0 | 0 | 0 |

TNTC = too numerous to count.

The silver composition proved to be surprisingly effective. Even at the shortest time (20 min) allowed for incubation of the lowest concentration tested (0.05 ppm) there was a complete kill of the bacteria. At 0.20 ppm and higher there was a complete kill at 5 minutes. It seems clear that a complete kill takes less than 5 minutes.

Evidence of Efficacy 32 ppm Silver as Surface Disinfectant

The Environmental Protection Agency (EPA) has approved a 32 ppm silver composition of the present invention as a broad spectrum surface disinfectant for use in hospitals, medical environments, residential homes, commercial buildings, and businesses. It has been approved for use against some of the most deadly pathogens including: Gram-positive bacteria, such as Staphylococcus aureus (presently considered to be the most deadly bacteria in U.S. hospitals), Gram-negative bacteria, such as Salmonella choleraesuis (responsible for food poisoning), and nosocomial or hospital-acquired pathogens, such as Pseudomonas aeruginosa (often found in burns and cuts).

Silver compositions of the present invention can be sprayed in and around occupied areas without endangering the health or wellness of humans or animals. One can disinfect surfaces selected from the group consisting of walls, tables, chairs, light fixtures, bathrooms, glass, porcelain, metal, glazed ceramic, enameled and painted by means of spraying or by means of wiping with a silver composition of the present invention. A preferred method of disinfecting comprises one or more of the steps of cleaning the surface to be disinfected, applying, by means of a spray, mop, sponge, or cloth, a composition of the present invention, thoroughly wetting the area to be disinfected, allowing the surface to remain wet for at least 10 minutes at a temperature of at least 20° C. (time/temperature interrelation can be adjusted via the Arrhenius equation or other means known to one of ordinary skill), and wiping the surface with a clean paper or cloth towel. Compositions for disinfecting surfaces comprise those comprising 5 to 40 ppm silver. A preferred composition of the present invention for disinfecting surfaces comprises (32±3) ppm silver. Another preferred composition of the present invention for disinfecting surfaces comprises (10±2) ppm silver. Another preferred composition of the present invention for disinfecting surfaces comprises (22±2) ppm silver.

Evidence of Efficacy of Silver Composition as Super Disinfectant

A. Purpose of Example

The purpose of this example is to show the antimicrobial activity of a silver composition of the present invention (here 10 ppm silver, 14 ppm silver with 1.5 wght % hydrogen peroxide, and 32 ppm silver) against the test organism Yersinia pestis, the etiologic agent of bubonic plague. By performing a standard kill-time assay using a Y. pestis suspension, it is demonstrated that silver compositions of the present invention are effective even against the bubonic plague bacteria.

B. Material and Methods

Y. Pestis, strain D27, was grown on a Columbia Agar plate for about 24 hours at 30° C. in a 5% CO₂ incubator. Growth from the plate was scraped into suspension, using 3 ml of sterile HPLC water. The suspension was transferred to a 50 ml conical centrifuge tube. The plate was then rinsed using an additional 2 ml of HPLC water. This rinse was added to the centrifuge tube. The tube was centrifuged at 3,500×g for 5 minutes. The supernatant was discarded and the pellet was resuspended in 1 ml of HPLC water, to give a final concentration of approximately 10¹⁰ cells per ml.

The method involved the following steps:

1. A 9.9 ml aliquot of the silver composition to be tested was placed in a sterile 20 mm×150 mm tube. The tube was equilibrated in a 20° C. water bath.

2. The tube of silver composition was inoculated with 100 [symbol] l of the test organism suspension at time zero to form a mixture. The tube was immediately vortexed and returned to the water bath.

3. At 2 min, 3 min, 4 min, and 5 min for 10 ppm or 32 ppm silver or 2 min, 4 min, 6 min and 8 min for 14 ppm silver with 1.5% v/v H₂O₂, 1 ml of organism/silver mixture was removed to 99 ml of neutralizer in a 250 ml Erlenmeyer flask. The flask was mixed thoroughly.

4. The neutralized suspension was immediately serially diluted 1:10 in physiological saline solution (PSS).

5. The number of viable organisms in selected dilution tubes and flasks was assayed by membrane filtration. One ml aliquots were plated in duplicate. The membranes were washed with about 150 ml (or 250 ml if the sample was taken from the neutralizer flask) of sterile phosphate buffered saline and removed to Columbia Agar plates. The entire remaining contents (98 ml) of the 4 and 5 min neutralizer flasks were also plated. The plates were incubated at 30° C. in a 5% CO₂ incubator for 72 hours.

6. The number of colonies on each filter was counted and log reductions were computed.

C. Results

The results for 10 ppm silver are shown in Table 7.

TABLE 7

| Time | Log Reduction | Percent Kill |
|---|---|---|
| 2 min | 2.63 | 99.77 |
| 4 min | 3.20 | 99.94 |
| 6 min | 3.46 | 99.97 |
| 8 min | 3.68 | 99.98 |

The calculated regression equation for these data is Y=2.3965+0.1696x. This indicates that the time for a 6-log reduction is 21.2 minute.

The results for 32 ppm silver are shown in Table 8.

TABLE 8

| Time | Log Reduction | Percent Kill |
|---|---|---|
| 2 min | >7.61 | 99.999998 |
| 4 min | >7.61 | 99.999998 |
| 6 min | >7.61 | 99.999998 |
| 8 min | >7.61 | 99.999998 |

The results for 14 ppm silver with 1.5% v/v H₂O₂ are shown in Table 9.

TABLE 9

| Time | Log Reduction | Percent Kill |
|---|---|---|
| 2 min | 3.27 | 99.95 |
| 3 min | 4.72 | 99.998 |
| 4 min | 5.36 | 99.9996 |
| 5 min | 6.47 | 99.99997 |

The calculated regression equation for these data is Y=1.371+1.024x. This indicates that the time for a 6-log reduction is 4.52 minute.

The silver composition of the present invention exhibited significant bactericidal activity against Y. pestis, the etiologic agent of bubonic plague. The 32 ppm composition gave more than a 7 log reduction (essentially total kill) in less than 2 min. The data show that the 10 ppm silver takes some 20 min to achieve a 6 log kill. The silver and hydrogen peroxide show significant synergism with a calculated 6 log kill of under 5 min. This is much better than 10 ppm silver alone. The level of 14 ppm silver was chosen because the data of other experiments suggested that this level of silver combined with hydrogen peroxide would achieve results approaching those of the 32 ppm silver product.

Data Summary

The following Table A contains a summary of the above results in terms of the effects of the inventive silver composition on a wide variety of microbes and human diseases. In some cases, the data presented in the table is not repeated above. However, the results were obtained using the procedures explained above so that one of ordinary skill in the art can readily replicated the results.

Human Diseases Cured By and Pathogens Killed by the Inventive Silver Composition

TABLE A

| Disease | Pathogen | Effective Concentration |
|---|---|---|
| Boils | Staphylococcus aureus | Killed @ 5 ppm |
| Osteomyelitis | Staphylococcus aureus | Killed @ 5 ppm |
| Bacillary Dysentery | Shigella boydii | Killed @ 2.5 ppm |
| Burn Infections | Pseudomonas aeruginosa | Killed @ 5 ppm |
| Dental Plaque | Streptococcus mutans | Killed @ 5 ppm |
| Diarrhea (Bloody) | Shigella boydii | Killed @ 2.5 ppm |
| Diarrhea | Escherichia coli | Killed @ 2.5 ppm |
| Ear Infection | Haemophilus influenzae | Killed @ 1.25 ppm |
| Ear Infection | Streptococcus pneumonie | Killed @ 2.5 ppm |
| Enteric Fever | Salmonella tyhimurium | Killed @ 2.5 ppm |
| Epiglottitis (In children) | Haemophilus influenzae | Killed @ 1.25 ppm |
| Eye Infections | Staphylococcus aureus | Killed @ 5 ppm |
| Corneal Ulcers-Keratitis | Pseudomonas aeruginosa | Killed @ 5 ppm |
| Food Poisoning | Salmonella arizona | Killed @ 5 ppm |
| Food Poisoning | Salmonella tyhimurium | Killed @ 2.5 ppm |
| Food Poisoning | Escherichia coli | Killed @ 2.5 ppm |
| Endocarditis | Streptococcus faecalis | Killed @ 2.5 ppm |
| Endocarditis | Streptococcus gordonii | Killed @ 5 ppm |
| Meningitis | Haemophilus influenzae | Killed @ 1.25 ppm |
| Meningitis | Enterobacter aerogenes | Killed @ 2.5 ppm |
| Meningitis | Pseudomonas aeruginosa | Killed @ 5 ppm |
| Meningitis | Streptococcus pneumonie | Killed @ 2.5 ppm |
| Nosocomial Infections | Klebsiella pneumoniae | Killed @ 2.5 ppm |
| Nosocomial Infections | Pseudomonas aeruginosa | Killed @ 5 ppm |
| Nosocomial Infections (From | Streptococcus pyogenes | Killed @ 1.25 ppm |
| hospitals) | | |
| Pneumonia | Staphylococcus aureus | Killed @ 5 ppm |
| Pneumonia | Haemophilus influenzae | Killed @ 1.25 ppm |
| Pneumonia | Pseudomonas aeruginosa | Killed @ 5 ppm |
| Pneumonia | Streptococcus pneumonie | Killed @ 2.5 ppm |
| Respiratory Tract Infections | Streptococcus pyogenes | Killed @ 1.25 ppm |
| Respiratory Tract Infections | E. coli | Killed @ 2.5 ppm |
| Respiratory Tract Infections | Klebsiella pneumoniae | Killed @ 2.5 ppm |
| Scarlet Fever | Streptococcus pyogenes | Killed @ 1.25 ppm |
| Septicemia | Enterobacter aerpyogenes | Killed @ 2.5 ppm |
| Sinus Infections | Haemophilus influenzae | Killed @ 1.25 ppm |
| Sinusitis | Streptococcus pneumonie | Killed @ 2.5 ppm |
| Impetigo | Staphylococcus aureus | Killed @ 1.25 ppm |
| Skin Infections | Staphylococcus aureus | Killed @ 5 ppm |
| Skin Infections | Streptococcus pyogenes | Killed @ 1.25 ppm |
| Strep Throat | Streptococcus pyogenes | Killed @ 1.25 ppm |
| Suppurative Arthritis | Haemophilus influenzae | Killed @ 1.25 ppm |
| Throat Infections | Haemophilus influenzae | Killed @ 1.25 ppm |
| Tooth Decay | Streptococcus mutans | Killed @ 5 ppm |
| Urethritis (Men) | Trichomonas vaginalis | Killed @ 10 ppm |
| Urinary Tract Infections | E. coli | Killed @ 2.5 ppm |
| Urinary Tract Infections | Klebsiella pneumoniae | Killed @ 2.5 ppm |
| Urinary Tract Infections | Pseudomonas aeruginosa | Killed @ 5 ppm |
| Urinary Tract Infections | Streptococcus faecalis | Killed @ 2.5 ppm |
| Urinary Tract Infections | Enterobacter aerpyogenes | Killed @ 2.5 ppm |
| Vaginitis (Women) | Trichomonas vaginalis | Killed @ 10 ppm |
| Wound Infections | Escherichia coli | Killed @ 2.5 ppm |
| Wound Infections | Enterobacter aerpyogenes | Killed @ 2.5 ppm |
| Wound Infections | Klebsiella pneumoniae | Killed @ 2.5 ppm |
| Wound Infections | Pseudomonas aeruginosa | Killed @ 5 ppm |
| Wound Infections | Streptococcus faecalis | Killed @ 2.5 ppm |
| Yeast Infections | Candida albicans | Killed @ 10 ppm |

Efficacy of Silver colloid Formulated as a Hydrogel

Modern wound care has come to recognize the fact that for optimal healing a wound should be kept sterile and protected from desiccation and environmental contaminants. Traditional bandages are effective as providing protection from environmental contaminants but are largely ineffective at preventing desiccation. Bandages may be rendered antimicrobial through the addition of a variety of disinfectant substances, but these substances are often harsh and kill cells or the body as well as microbes. In recent times wound care has been revolutionized by hydrogel materials which are available as either a semisolid (amorphous material) or as a soft sheet-like material. The hydrogel is hydrophilic and hence prevents desiccation of the wound. The sheet-like material is effective at excluding environmental contaminants and because of its hydrophilic character, the hydrogel can actually absorb excess fluid exuded by the wound.

Hydrogels are formed by combining a hydrophilic polymer with other ingredients in an aqueous solution. The polymer forms a gel following a change in pH, temperature or other triggering event. In a gel a fine molecular network of the polymer surrounds regions of the aqueous solution. Although the composition may be an amorphous semi-solid or a firmer sheet-like material, the vast majority of the volume tends to be occupied by the aqueous solution as opposed to the hydrophilic polymer. Hydrophilic polymers that are appropriate for the production of hydrogels include gelatin, carboxy-methyl cellulose (and other cellulose derivatives), other carbohydrate polymers of plant or algal origin such as alginate, carrageenan, xanthan gum, locust bean gum, gum traganth, guar gum, gum arabic and other plant gums, acrylic acid copolymers (such as Carbopol), and combinations of these and similar hydrophilic polymers.

The aqueous component preferably contains various additive substances that enhance the physical characteristics of the hydrogel and/or enhance wound healing. These include various vitamins, amino acids and growth factors added to enhance healing or reduce scar formation to diminish scarring. Common anesthetics such as novocaine, lidocaine and derivatives thereof can also be incorporated as additives to enhance comfort. Since keeping the wound sterile is a major goal of the dressing, various antimicrobial or disinfectant agents are advantageously included. These include organic acids such as citric acid, dilute acetic acid, benzoic acid, proprionic acid and lactic acid. Alcohols such as isopropanol or ethanol are useful as are organic disinfectants including chlorinated phenolics such as “TCP”(2,4,6 trichlorophenol), biguanides, chlorhexidine (when mixed with cetrimide), chlorhexidine gluconate, and chlorhexidine acetate. Disinfectant surfactants including amphotheric surfactants and aldehydes such as formaldehyde and glutaraldehyde can be included. Halogen disinfectants including iodine, iodophores, and polyvidone-iodine are effective as are peroxides and other oxygenators such as hydrogen peroxide. Other beneficial ingredients include aluminum-zinc astringent agents, furan derivatives and quinoline derivatives such as clioquinol. As beneficial as all these antimicrobial agents may be, they all tend to suffer from the defect that they can be damaging to tissue and/or microbes can readily develop resistance to them.

As amply demonstrated above, the inventive silver colloid is highly effective antimicrobially, is very gentle to human tissue and is effective against resistant microbes. Both amorphous gel and hydrogel sheet are both amenable to delivering effective levels of colloidal silver in moist healing environment. On one hand the amorphous hydrogel slowly releases colloidal silver as it slowly softens in tissue exudate and gradually begins to dissolve. On the other hand amorphous hydrogel donates moisture to the tissue and simultaneously makes colloidal silver available at site. In addition, a small amount of colloidal silver present in the dressing has the advantage of being molecular silver, whose gradual reduction over an extended period of time will release silver ions which have excellent oligodynamic activity.

After initial experiments Carbopol was selected as an effective hydrogel forming agent for use with the inventive colloidal silver. A basic formulation was developed which generally included the following ingredients as shown in Table 9a.

TABLE 9a

| Ingredient | Function | Supplier |
|---|---|---|
| Colloidal Silver Solution | Active, Anti-microbial and | American |
| (22 ppm or 32 ppm) | Diluent | Biotech Labs |
| Carbopol ETD2020 | Rheology Modifier | Noveon |
| Triethanolamine | Neutralizer, Penetrating | E. Merck |
| | agent | |
| Propylene Glycol | Humectant | E. Merck |

All raw materials were first analyzed for

1. Anti-bacterial Activity

2. Physical and Chemical Properties:

1. Appearance
2. Odor
3. pH
4. Feel
5. Density
6. Foaming Property
7. Flow-ability.

Colloidal Silver Solution (22 ppm or 32 ppm):

In this formulation Silver Solution is used as an active component (anti-microbial agent). It is also the only diluent in this specific formulation.

A. Anti-Bacterial Activity:

Diameter of zone of inhibition

| Culture | 22 ppm | 32 ppm |
|---|---|---|
| MRSA | 17 mm | 18 mm |
| E. coil | 14 mm | NA |
| Ps. aeruginosa | 21 mm | 22 mm |

B. Physical and Chemical Properties:

| | | |
|---|---|---|
| 1. | Appearance | Colorless clear liquid |
| 2. | Odor | Odorless |
| 3. | pH | 5.0 |
| 4. | Feel | Not Applicable |
| 5. | Density | 1.00 |
| 6. | Foaming Property | Not Applicable |
| 7. | Flow-ability | Not Applicable |

Carbopol

Carbopol is chemically known as carboxypolymethylene or carboxyvinyl polymer. It is a copolymer of acrylic acid and is highly ionic (i.e., hydrophylic) and slightly acidic compound. Carbopol polymers must be neutralized in order to achieve maximum viscosity. It is used in pharmaceuticals, cosmetic and textile printing fields as a thickening, suspending, dispersing and emulsifying agent. In this formulation Carbopol is used as a gelling or thickening agent.

A. Anti-Bacterial Activity Not Applicable

B. Physical and Chemical Properties:

| | |
|---|---|
| 1. Appearance | Dry, white powder |
| 2. Odor | Odorless |
| 3. pH | Not Applicable |
| 4. Feel | Not Applicable |
| 5. Density | Not Applicable |
| 6. Foaming Property | Not Applicable |
| 7. Flow-ability | Not Applicable |

Triethanolamine

(TEA) C₆H₁₅NO₃ (Mol. Wt.: 149.19)

In this formulation Triethanolamine, an alkalizing agent neutralizes Carbopol to raise the viscosity. It also increases the penetrating power of the active agent.

A. Anti-Bacterial Activity (Not Applicable)

B. Physical and Chemical Properties

| | |
|---|---|
| 1. Appearance | Colorless viscous liquid |
| 2. Odor | Slight ammoniacal |
| 3. pH | Not Applicable |
| 4. Feel | Not Applicable |
| 5. Density | 1.1242 g/cc |
| 6. Foaming Property | Not Applicable |
| 7. Flow-ability | Not Applicable |

Propylene Glycol

C₃H₈O₂ Mol. Wt.: 76.09

Propylene Glycol is chemically known as 1:2 propanediol. It is used as a humectant and feel modifier in this formulation.

A. Anti-Bacterial Activity Not Applicable

B. Physical and Chemical Properties:

| | |
|---|---|
| 1. Appearance | Colorless viscous liquid |
| 2. Odor | Odorless |
| 3. pH | Not Applicable |
| 4. Feel | Not Applicable |
| 5. Density | 1.036 gm/cc |
| 6. Foaming Property | Not Applicable |
| 7. Flow-ability | Not Applicable |

Once the standard formula was developed a number of batches were manufactured to explore the possible range of formulations. From the 19 experiments carried out the following observation were reached.

1. Increase in pH increases viscosity of gel.

2. Increase in Carbopol quantity increases viscosity of gel.

3. Higher the Carbopol percentage the higher the tackiness.

From the above experiments it can be concluded that a trade off has to be reached between amount of Carbopol and TEA used and the final pH obtained which should not be more than 8.5. Hence formulation No. 18 was kept as standard and batch scaled up to 10 Kg.

Produce Development Studies Introduction:

Carbopol based gel formulations have to be standardized with respect to pH, feel, tackiness and consistency. With this in mind various lab batches were taken using water as the aqueous phase to obtain a product of suitable quality and feel before taking the main batch.

Batch No. SG/001 Formulation:

| | | | |
|---|---|---|---|
| Part A: | Distilled Water | 83.50 | g |
| | Carbopol | 00.62 | g |
| | NaOH 18% | 00.60 | g |
| Part B: | Distilled Water | 1.00 | g |
| | Propylene glycol | 5.00 | g |
| | NaOH 18% | 1.50 | g |

Procedure: Weigh the given amount of Distilled Water from Part A and keep in water bath at 70° C. Add Carbopol to Distilled Water with constant stirring to avoid lumps. Add NaOH 18% to it at 70° C. after 20 minutes. Weigh all the ingredients from Part B and keep in water bath at 70° C. for 15-20 min. Add Part B to Part A and stir it for 10-15 min. Cool it to room temperature and analyze.

Results:

| | | |
|---|---|---|
| 1. pH | | 10.8 |
| 2. Flow-ability | 90° C. => | >5 min. |
| | 45° C. => | >5 min. |
| 3. Tackiness | | Very Tacky |

Batch No. SG/002 Formulation:

| | | | |
|---|---|---|---|
| Part A: | Distilled water | 83.50 | g |
| | Carbopol | 00.62 | g |
| | TEA | 01.20 | g |
| Part B: | Distilled water | 1.0 | g |
| | Propylene glycol | 5.0 | g |
| | TEA | 1.5 | gm |

Procedure: Weigh the given amount of Distilled water from part A and keep in water bath at 70° C. Add Carbopol to Distilled water with constant stirring to avoid lumps. Add TEA to it at 70° C. after 20 minutes. Weigh all the ingredients from part B and keep in water bath at 70° C. for 15-20 min. Add part B to part A and stir it for 10-15 min. Cool it to room temperature and analyze.

Results:

| | | |
|---|---|---|
| 1. pH | | 7.9 (SOP-08) |
| 2. Flow-ability | 90° C. => | >5 min. |
| | 45° C. => | >5 min. |
| 3. Tackiness | | Very Tacky |

Batch No. SG/003 Formulation:

| | | | |
|---|---|---|---|
| Part A: | Distilled water | 86.00 | g |
| | Carbopol | 00.62 | g |
| | TEA | 01.20 | g |
| Part B: | Distilled water | 2.00 | g |
| | Propylene glycol | 5.00 | g |
| | TEA | 1.50 | g |

Procedure: Weigh the given amount of Distilled water from part A and keep in water bath at 70° C. Add Carbopol to Distilled water with constant stirring to avoid lumps. Add TEA to it at 70° C. after 20 minutes. Weigh all the ingredients from part B and keep in water bath at 70° C. for 15-20 min. Add part B to part A and stir it for 10-15 min. Cool it to room temperature and analyze.

| | | |
|---|---|---|
| 1. pH | | 8.62 |
| 2. Flow-ability | 90° C. => | >5 min. |
| | 45° C. => | >5 min. |
| 3. Tackiness | | Very Tacky |

Results:

Batch No. SG/004 Formulation:

| | | | |
|---|---|---|---|
| Part A: | Distilled water | 86.00 | g |
| | Carbopol | 00.62 | g |
| | TEA | 01.00 | g |
| Part B: | Distilled water | 2.00 | g |
| | Propylene glycol | 5.00 | g |
| | TEA | 1.50 | g |

Procedure: Weigh the given amount of Distilled water from Part A and keep in water bath at 70° C. Add Carbopol to Distilled Water with constant stirring to avoid lumps. Add TEA to it at 70° C. after 20 minutes. Weigh all the ingredients from Part B and keep in water bath at 70° C. for 15-20 min. Add Part B to Part A and stir it for 10-15 min. Cool it to room temperature and analyze.

Results:

| | | |
|---|---|---|
| 1. pH | | 8.5 |
| 2. Flow-ability | 90° C. => | >5 min. |
| | 45° C. => | >5 min. |
| 3. Tackiness | | Very Tacky |

Batch No. SG/005 Formulation:

| | | | |
|---|---|---|---|
| Part A: | Distilled water | 86.0 | g |
| | Carbopol | 0.62 | |
| | TEA | 1.20 | g |
| Part B: | Distilled water | 2.00 | g |
| | Propylene glycol | 7.00 | g |
| | TEA | 1.50 | g |

Procedure: Weigh the given amount of Distilled Water from Part A and keep in water bath at 70° C. Add Carbopol to Distilled Water with constant stirring to avoid lumps. Add TEA to it at 70° C. after 20 minutes. Weigh all the ingredients from Part B and keep in water bath at 70° C. for 15-20 min. Add Part B to Part A and stir it for 10-15 min. Cool it to room temperature and analyze.

Results:

| | | |
|---|---|---|
| 1. pH | | 8.7 |
| 2. Flow-ability | 90° C. => | >5 min. |
| | 45° C. => | >5 min. |
| 3. Tackiness | | Very Tacky |

Batch No. SG/006 Formulation:

| | | | |
|---|---|---|---|
| Part A: | Distilled water | 85.00 | g |
| | Carbopol | 00.62 | g |
| | TEA | 01.00 | g |
| Part B: | Distilled water | 1.00 | g |
| | Propylene glycol | 5.00 | g |
| | TEA | 1.40 | g |

Procedure: Weigh the given amount of Distilled Water from Part A and keep in water bath at 70° C. Add Carbopol to Distilled Water with constant stirring to avoid lumps. Add TEA to it at 70° C. after 20 minutes. Weigh all the ingredients from Part B and keep in water bath at 70° C. for 15-20 min. Add Part B to Part A and stir it for 10-15 min. Cool it to room temperature and analyze.

Results:

| | | |
|---|---|---|
| 1. pH | | 8.4 |
| 2. Flow-ability | 90° C. => | >5 min. |
| | 45° C. => | >5 min. |
| 3. Tackiness | | Very Tacky |

Batch No. SG/007 Formulation:

| | | | |
|---|---|---|---|
| Part A: | Distilled Water | 172 | g |
| | Carbopol | 1.24 | g |
| | TEA | 2.40 | g |
| Part B: | Distilled Water | 6.0 | g |
| | Propylene glycol | 10 | g |
| | TEA | 2.80 | g |

Procedure: Weigh the given amount of Distilled Water from Part A and keep in water bath at 70° C. Add Carbopol to Distilled Water with constant stirring to avoid lumps.

Add TEA to it at 70° C. after 20 minutes. Weigh all the ingredients from Part B and keep in water bath at 70° C. for 15-20 min. Add Part B to part A and stir it for 10-15 min. Cool it to room temperature and analyze.

Results:

| | | |
|---|---|---|
| 1. | pH | 8.28 |
| 2. | Flow-ability | 90° C. => >5 min. |
| | | 45° C. => >5 min. |
| 3. | Tackiness | Very Tacky |

Batch No. SG/008 Formulation:

| Part A: | Silver Solution (32 ppm) | 86 | g |
|---|---|---|---|
| | Carbopol | 0.62 | g |
| | TEA | 1.20 | g |
| Part B | Silver Solution (32 ppm) | 2.0 | g |
| | Propylene glycol | 5.0 | |
| | TEA | 1.5 | g |

Procedure: Weigh the given amount of Silver Solution from part A and keep in water bath at 70° C. Add Carbopol to Silver Solution with constant stirring to avoid lumps. Add TEA to it at 70° C. after 20 minutes. Weigh all the ingredients from Part B and keep in water bath at 70° C. for 15-20 min. Add Part B to part A and stir it for 10-15 min. Cool it to room temperature and analyze.

Results:

| | | |
|---|---|---|
| 1. | pH | 8.65 |
| 2. | Flow-ability | 90° C. => >5 min. |
| | | 45° C. => >5 min. |
| 3. | Tackiness | Very Tacky |

Batch No. SG/009 Formulation:

| | | | |
|---|---|---|---|
| Part A: | Silver Solution (32 ppm) | 172 | g |
| | Distilled Water | 12 | g |
| | Carbopol | 1.24 | g |
| | TEA | 2.40 | g |
| Part B: | Silver Solution (32 ppm) | 6.00 | g |
| | Propylene glycol | 10.0 | g |
| | TEA | 2.80 | g |

Procedure: Weigh the given amount of Silver Solution from part A and keep in water bath at 70° C. Add Carbopol to Silver Solution with constant stirring to avoid lumps. Add TEA to it at 70° C. after 20 minutes. Weigh all the ingredients from Part B and keep in water bath at 70° C. for 15-20 min. Add Part B to part A and stir it for 10-15 min. Cool it to room temperature and analyze.

Results:

| | | |
|---|---|---|
| 1. | pH | 8.54 |
| 2. | Flow-ability: | 90° C. => >5 min. |
| | | 45° C. => >5 min. |
| 3. | Tackiness | Very Tacky |

Batch No. SGI010 Formulation:

| | | | |
|---|---|---|---|
| Part A: | Silver Solution (32 ppm) | 172 | g |
| | Distilled Water | 24 | g |
| | Carbopol | 1.39 | g |
| | TEA | 2.40 | g |
| Part B: | Silver Solution (32 ppm) | 6.00 | g |
| | Propylene glycol | 5.00 | g |
| | TEA | 2.80 | g |

Procedure: Weigh the given amount of Silver Solution from part A and keep in water bath at 70° C. Add Carbopol to Silver Solution with constant stirring to avoid lumps. Add TEA to it at 70° C. after 20 minutes. Weigh all the ingredients from Part B and keep in water bath at 70° C. for 15-20 min. Add Part B to part A and stir it for 10-15 min. Cool it to room temperature and analyze.

Results:

| | | |
|---|---|---|
| 1. | pH | 8.43 |
| 2. | Flow-ability | 90° C. => >5 min. |
| | | 45° C. => >5 min. |
| 3. | Tackiness | Tacky |

Batch No. SG/011 Formulation:

| | | | |
|---|---|---|---|
| Part A: | Distilled Water | 98 | g |
| | Carbopol | 0.76 | g |
| | TEA | 0.56 | g |
| Part B: | Distilled Water | 3.0 | g |
| | Propylene glycol | 5.0 | g |
| | TEA | 1.4 | g |

Procedure: Weigh the given amount of Distilled Water from Part A and keep in water bath at 70° C. Add Carbopol to Distilled Water Solution with constant stirring to avoid lumps. Add TEA to it at 70° C. after 20 minutes. Weigh all the ingredients from Part B and keep in water bath at 70° C. for 15-20 min. Add Part B to Part A and stir it for 10-15 min. Cool it to room temperature and analyze.

Results:

| | | |
|---|---|---|
| 1. | pH | 8.05 |
| 2. | Flow-ability | 90° C. => >5 min. |
| | | 45° C. => >5 min. |
| 3. | Tackiness | Very Tacky |

Batch No. SG1012 Formulation:

| | | | |
|---|---|---|---|
| Part A | Distilled water | 98 | g |
| | Carbopol | 0.76 | g |
| | TEA | 0.34 | g |
| Part B | Distilled Water | 3.00 | g |
| | Propylene glycol | 5.00 | g |
| | TEA | 0.64 | g |

Procedure: Weigh the given amount of Distilled Water from Part A and keep in water bath at 70° C. Add Carbopol to Distilled Water Solution with constant stirring to avoid lumps. Add TEA to it at 70° C. after 20 minutes. Weigh all the ingredients from Part B and keep in water bath at 70° C. for 15-20 min. Add Part B to Part A and stir it for 10-15 min. Cool it to room temperature and analyze.

Results

| | | |
|---|---|---|
| 1. | pH | 6.35 |
| 2. | Flow-ability | 90° C. => >5 min. |
| | | 45° C. => >5 min. |
| 3. | Tackiness | Very Tacky |

Batch No. SG/013 Formulation:

| | | | |
|---|---|---|---|
| Part A: | Silver Solution (32 ppm) | 86 | g |
| | Distilled Water | 12 | g |
| | Carbopol | 0.76 | g |
| | TEA | 0.32 | g |
| Part B: | Silver Solution (32 ppm) | 3.00 | g |
| | Propylene glycol | 5.00 | g |
| | TEA | 0.64 | g |

Procedure: Weigh the given amount of Silver Solution and Distilled Water from Part A and keep in water bath at 70° C. Add Carbopol to Solution with constant stirring to avoid lumps. Add TEA to it at 70° C. after 20 minutes. Weigh all the ingredients from Part

B and keep in water bath at 70° C. for 15-20 min. Add Part B to Part A and stir it for 10-15 min. Cool it to room temperature and analyze.

Results:

| | | |
|---|---|---|
| 1. | pH | 6.7 |
| 2. | Flow-ability | 90° C. => >5 min. |
| | | 45° C. => >5 min. |
| 3. | Tackiness | Very Tacky |

Batch No. SG/014 Formulation:

| | | | |
|---|---|---|---|
| Part A: | Silver Solution (32 ppm) | 86 | g |
| | Distilled Water | 12 | g |
| | Carbopol | 0.78 | g |
| | TEA | 0.32 | gm |
| Part B | Silver Solution (32 ppm): | 3.00 | g |
| | Propylene glycol | 5.00 | g |
| | TEA | 0.64 | g |

Procedure: Weigh the given amount of Silver Solution and Distilled Water from Part A and keep in water bath at 70° C. Add Carbopol to Solution with constant stirring to avoid lumps. Add TEA to it at 70° C. after 20 minutes. Weigh all the ingredients from Part B and keep in water bath at 70° C. for 15-20 min. Add Part B to Part A and stir it for 10-15 min. Cool it to room temperature and analyze.

Results

| | |
|---|---|
| 1. pH | 6.6 |
| 2. Flow-ability | 90° C. => >5 min. |
| | 45° C. => >5 min. |
| 3. Tackiness | Very Tacky |

Batch No. SG/015 Formulation:

| | | | |
|---|---|---|---|
| Part A | Silver Solution (32 ppm) | 86 | g |
| | Distilled Water | 12 | g |
| | Carbopol | 0.68 | g |
| | TEA | 0.40 | g |
| Part B | Silver Solution (32 ppm) : | 5.0 | g |
| | Propylene glycol | 7.0 | g |
| | TEA | 0.6 | g |

Procedure: Weigh the given amount of Silver Solution and Distilled Water from

Part A and keep in water bath at 70° C. Add Carbopol to Solution with constant stirring to avoid lumps. Add TEA to it at 70° C. after 20 minutes. Weigh all the ingredients from Part B and keep in water bath at 70° C. for 15-20 min. Add Part B to Part A and stir it for 10-15 min. Cool it to room temperature and analyze.

Results

| | |
|---|---|
| 1. pH | 6.72 |
| 2. Row-ability | 90° C. => >5 min. |
| | 45° C. => >5 min |
| 3. Tackiness | Tacky |

Batch No. SG/016 Formulation:

| | | | |
|---|---|---|---|
| Part A: | Silver Solution (32 ppm) | 86 | g |
| | Distilled Water | 12 | g |
| | Carbopol | 0.64 | g |
| | TEA | 0.40 | g |
| Part B: | Silver Solution (32 ppm) | 5.0 | g |
| | Propylene glycol | 7.0 | g |
| | TEA | 0.6 | g |

Procedure: Weigh the given amount of Silver Solution and Distilled Water from

Part A and keep in water bath at 70° C. Add Carbopol to Solution with constant stirring to avoid lumps. Add TEA to it at 70° C. after 20 mins. Weigh all the ingredients from Part B and keep in water bath at 70° C. for 15-20 min. Add Part B to Part A and stir it for 10-15 min. Cool it to room temperature and analyze.

Results:

| | |
|---|---|
| 1. pH | 6.87 |
| 2. Flow-ability | 90° C. => >5 min. |
| | 45° C. => >5 min |
| 3. Tackiness | Tacky |

Batch No. SG/017 Formulation:

| | | | |
|---|---|---|---|
| Part A: | Silver Solution (32 ppm) | 86 | g |
| | Distilled Water | 12 | g |
| | Carbopol | 0.62 | g |
| | TEA | 0.4 | g |
| Part B: | Silver Solution (32 ppm) | 5.0 | g |
| | Propylene glycol | 7.0 | g |
| | TEA | 0.6 | g |

Procedure: Weigh the given amount of Silver Solution and Distilled Water from Part A and keep in water bath at 70° C. Add Carbopol to Solution with constant stirring to avoid lumps. Add TEA to it at 70° C. after 20 minutes. Weigh all the ingredients from Part B and keep in water bath at 70° C. for 15-20 min. Add Part B to Part A and stir it for 10-15 min. Cool it to room temperature and analyze.

Results:

| | |
|---|---|
| 1. pH | 7.05 |
| 2. Flow-ability | 90° C. => >5 min. |
| | 45° C. => >5 min |
| 3. Tackiness | Tacky |

Batch No. SG/018 Formulation:

| | | | |
|---|---|---|---|
| Part A: | Silver Solution (32 ppm) | 86 | g |
| | Distilled Water | 12 | g |
| | Carbopol | 0.58 | g |
| | TEA | 0.4 | g |
| Part B: | Silver Solution (32 ppm) | 5.0 | g |
| | Propylene glycol | 7.0 | g |
| | TEA | 0.6 | g |

Procedure: Weigh the given amount of Silver Solution and Distilled Water from Part A and keep in water bath at 70° C. Add Carbopol to Solution with constant stirring to avoid lumps. Add TEA to it at 70° C. after 20 minutes. Weigh all the ingredients from Part B and keep in water bath at 70° C. for 15-20 min. Add Part B to Part A and stir it for 10-15 min. Cool it to room temperature and analyze.

Results:

| | |
|---|---|
| 1. pH | 7.40 |
| 2. Flow-ability | 90° C. => >5 min. |
| | 45° C. => >5 min |
| 3. Tackiness | Smooth |

Batch No. SG/019 Formulation:

| | | | |
|---|---|---|---|
| Part A: | Silver Solution (32 ppm) | 86 | g |
| | Distilled water | 12 | g |
| | Carbopol | 0.54 | g |
| | TEA | 0.4 | g |
| Part B: | Silver Solution (32 ppm) | 5.0 | g |
| | Propylene glycol | 7.0 | g |
| | TEA | 0.6 | g |

Procedure: Weigh the given amount of Silver Solution and Distilled Water from Part A and keep in water bath at 70° C. Add Carbopol to Solution with constant stirring to avoid lumps. Add TEA to it at 70° C. after 20 minutes. Weigh all the ingredients from Part B and keep in water bath at 70° C. for 15-20 min. Add Part B to Part A and stir it for 10-15 min. Cool it to room temperature and analyze.

Results:

| | |
|---|---|
| 1. pH | 7.65 |
| 2. Flow-ability | 90° C. => 1 min. |
| | 45° C. => 2 min |
| 3. Tackiness | Smooth |

Remark: Though the Gel feel has improved consistency is not suitable.

Based on the above results the following instructions for a one kilogram batch were developed.

| | | |
|---|---|---|
| Part A | Silver Solution | 860 gm |
| | Distilled water | 100 gm |
| | Carbopol | 5.80 gm |
| | TEA | 4.00 gm |
| Part B | ASAP Solution | 50.0 gm |
| | Propylene glycol | 70.0 gm |
| | TEA | 6.00 gm |
| | | Yield 1.0 Kg. after adjusting for moisture loss. |

Procedure: In a clean sterilized vessel take the required quantity of Distilled Water and silver solution. Raise the temperature of solution to 70° C. with continuous stirring. Start addition of Carbopol in minute amounts with continuous stirring/homogenization. After all Carbopol has been added continue for 30 minutes. (Adjust time according to batch size). Then add TEA into the phase A solution.

In a separate vessel mix all ingredients of part B. Raise the temperature to 70° C. and slowly add part B to Part A. On complete homogenization cool it to room temperature.

Precautions: Carbopol dispersion must be done using a good homogenizer. Take a small trial batch when using a new lot of Carbopol. Minimize Heating time as longer heating leads to more water loss.

Results

| | |
|---|---|
| 1. pH | 7.4 |
| 2. Flow-ability | >5 min. |
| 3. Tackiness | Smooth. |

This formulation has been readily scaled up to 10 kgs. in a pilot plant. No problems were encountered during scale up. Deaeration by vacuum application is recommended to remove entrapped air and ensure uniform filling.

This formulation has the following physical and chemical characteristics as shown in Table 10.

TABLE 10

| | TEST | SPECIFICATION | RESULTS |
|---|---|---|---|
| 1. | Appearance | Golden yellow | Passes |
| | | Translucent Gel | |
| 2. | Odor | Odorless | Odorless |
| 3. | Specific Gravity | 1.02 | 1.02 |
| 4. | Flowability | At 45° and 90°—More than 5 | At 45° and 90°— |
| | | min. to travel 1 inch from | More than 5 min. |
| | | the origin | to travel 1 inch |
| | | | from the origin |
| 5. | Foaming Cap. | <10 ml | <10 ml |
| 6. | Feel/Tackiness | 1-Smooth | 1-Smooth |
| 7 | Viscosity RT 30° | 32,000 ± 5000 | 34,000 |
| | 370 | 30,000 ± 5000 | 33,500 |
| 8. | PH | 6.5 to 8.0 | 7.4 |
| 9. | Freeze and Thaw | To pass SOP 1-10 | Compares with |
| | | | Original |
| 10. | Optimum | 22 ppm − 400 +/− 20 nm. | 400 nm. ** |
| | Wavelength (X | 32 ppm − 450 +/− 20 nm. | 450 nm. ** |
| | Max) | | |
| 11. | Light Exposure | No further discoloration | Passes. |
| 12. | Compatibility | No discoloration of | Ref Table 3 |
| | | product I reaction with | |
| | | containers. | |
| 13. | Moisture Donation | — | 10.27% |
| 14. | Moisture Uptake | — | 80% |

Microbiological Evaluation

It is reasonable to assume that the silver colloid hydrogel has microbiological proprieties similar to the original silver colloid which has been extensively tested as demonstrated above. However, the addition of the hydrophilic polymer to produce the gel might directly interfere with the microbial properties of the silver or might so inhibit diffusion of the silver that effectiveness is decreased. Therefore, microbiological tests similar to those carried out on the silver colloid solution were also performed on the silver colloid hydrogel.

Initially, the hydrogel was tested to determine whether the composition was self-sterilizing. The following protocol was followed:

Flasks of 100 ml sterile Fluid Thioglycollate Medium (Anaerobic bacteria), sterile Soya bean Casein Digest Medium (Aerobic bacteria), and Potato Dextrose Broth (Fungi) were obtained Samples of about 100 mg of gel to be tested were aseptically transferred into sets of flask. One set was incubated at 37° C. and another set was incubated at room temperature for one week. After that time the flasks were inspected and showed no turbidity or sign of microbial growth. Because the gel sample had not been manufactured under sterile conditions, it can be concluded that the composition is self-sterilizing. The100 mg of gel used for each test This corresponds to 2.2 μg in 100 ml medium or 0.02214 or 0.032 μg of silver per ml of medium. At this concentration silver would not have antimicrobial activity and hence false negative results can be eliminated.

A variety of test organisms were then used to compare the zone of inhibition attained with either 22 or 32 ppm silver solution or 22 or 32 ppm silver gel made as described above and as shown in Table 11. Aliquots of 0.1 ml or actively growing 18 hr cultures of each microorganism (approximately 10⁸ CFU/ml) were spread on sterile nutrient agar plates. A 10 mm diameter hole was punched in each inoculated plate with a cork borer. A test amount of (0/2-0.3 g) of the product was placed into each hole, and the plate was incubated for 24 hr. After that time the plates were inspected and the following zones of inhibition (total diameter of each zone) were measured.

TABLE 11

| | Silver Solution | | Silver Gel | |
|---|---|---|---|---|
| Culture | 22 ppm | 32 ppm | 22 ppm | 32 ppm |
| E. coil | 14 mm | 14 mm | 12 mm | 13 mm |
| Ps. Aeruginosa | 21 mm | 22 mm | 21 mm | 20 mm |
| B. subtilis | 15 mm | 16 mm | 14 mm | 14 mm |
| MRSA 1 | 17 mm | 18 mm | 16 mm | 17 mm |
| MRSA 2 | 16 mm | 17 mm | 16 mm | 17 mm |
| S. aureus | 14 mm | 14.5 mm | 15 mm | 15 mm |
| ATCC 6538 P | | | | |
| S. pyogenes | 16 mm | 18 mm | 16 mm | 18 mm |
| S.typhi | 17 mm | 16 mm | 16 mm | 16 mm |
| Sh. flexneri | 20 mm | 21 mm | 20 mm | 21 mm |
| K. pneumoniae | 17 mm | 18 mm | 18 mm | 18 mm |
| C. diptheriae | 16 mm | 18 mm | 16 mm | 17 mm |
| C. albicans | 39 mm | 40 mm | 39 mm | 40 mm |

These results show that the inhibitory effects of the gel are essentially equivalent to those of the silver colloid solution; this demonstrates that the gelling polymer does not negatively affect the antimicrobial powers of the silver colloid. Some cultures (S. pyogenes, C. diphtheriae and S. aureus) were also cultured on Blood Agar. The results suggested that the silver gel would also be effective on a bloody, exuding wound.

Similar tests were carried out on the same bacterial strains using a variety of antibiotic agents. In some cases the antibiotics were more effective than the silver compounds—in others they were much less effective. This demonstrates that the strains used were not weakened or “push-over” strains (see Table 12a and 12b).

TABLE 12a
Gram Positive Bacteria

| Antibiotic | Conc. | S.aureus | MRSA 1 | | MRSA 2 | | B. subtilis |
|---|---|---|---|---|---|---|---|
| Ampicillin | 200 mcg | Clear | 15 | mm | 18 | mm | 16 mm |
| Cefotaxime | 30 mcg | 26 mm | No | | -Clear | | 12 mm |
| | | | inhibition | | | | |
| Cephalexin | 30 mcg | Clear | 1.0 | mm | 0.8 | mm | Clear |
| Ciprofloxacin | 5 mcg | 28 mm | 14 | mm | 14 | mm | 20 mm |
| Cloxacillin | 1 mcg | Clear | Clear | | 13 | mm | 18 mm |
| Co- | 25 mcg | Clear | No | | No | | 15 mm |
| Trimoxazole | | | inhibition | | inhibition | | |
| Gentamycin | 10 mcg | 1Clear | No | | 11 | mm | 18 mm |
| | | | inhibition | | | | |
| Lincomycin | 2 mcg | 1Clear | Clear | | Clear | | 18 mm |
| Ofloxacin | 5 mcg | Clear | 15 | mm | 16 | mm | 22 mm |
| Peflofloxacin | 10 mcg | 30 mm | 11 | mm | 13 | mm | 21 mm |
| Roxythromycin | 15 mcg | Clear | 1.0 | mm | 12 | mm | 20 mm |

| | | | | | | |
|---|---|---|---|---|---|---|
| Tetracyclin | 30 mcg | 34 mm | No | 0.7 | mm | 19 mm |
| | | | inhibition | | | |

TABLE 12b
Gram Negative Bacteria

| | | | K. pneu- | | Ps. |
|---|---|---|---|---|---|
| Antibiotics | Conc. | E.coli | moniae | S.typhi | aeruginosa |
| Amikacin | 30 mcg | Clear | 18 mm | Clear | 10 mm |
| Ampicillin | 200 mcg | 23 mm | 18 mm | 20 mm | 13 mm |
| Cefotaxime | 30 mcg | 21 mm | 20 mm | 22 mm | 19 mm |
| Ceftizoxime | 30 mcg | 18 mm | 18 mm | 15 mm | No inhibition |
| Chloramphenicol | 30 mcg | 22 mm | 21 mm | 23 mm | No inhibition |
| Ciprofloxacin | 5 mcg | 29 mm | 22 mm | 25 mm | 15 mm |
| Co- | 25 mcg | 24 mm | 19 mm | 27 mm | Clear |
| Trimoxazole | | | | | |
| Gentamycin | 10 mcg | Clear | 17 mm | Clear | No inhibition |
| Ofloxacin | 5 mcg | Clear- | 29 mm | clear | 15 mm |
| Pefloxacin | 10 mcg | Clear | 25 mm | clear | 10 mm |
| Piperacillin | 100 mcg | 22 mm | 15 mm | 16 mm | 10 mm |
| Tetracyclin | 30 mcg | 19 mm | 18 mm | 16 mm | No inhibition |

Hand Scrub Test

Since the hydrogel has the ability to increase the adherence of silver to skin surfaces, effectiveness of the gel as a hand scrub was evaluated. For this test a one inch square of a volunteers hand was marked and then scrubbed with about 1 g of the gel. A control area was scrubbed with sterile distilled water. The areas were swabbed and the swab streaked on nutrient agar. The swabbing was repeated every hour for four hours. The streaked plates were incubated for 24 hr at 37° C. and the results evaluated.

As shown in the following Table 13, the control swabs grew so many bacteria as to be Too Numerous To Count (TNTC). The areas treated with silver gel remained essentially sterile for three hours and showed only slight growth at four hours. This should provide superior results for health care workers who need to sterilize the surface of their hands without using harsh or irrigating compounds.

TABLE 13

| Time | Control | 22 ppm | 32 ppm |
|---|---|---|---|
| 0 hr | TNTC | No growth | No growth |
| 1 hr | TNTC | No growth | No growth |
| 2 hr | TNTC | No growth | No growth |
| 3 hr | TNTC | No growth | No growth |
| 4 hr | TNTC | 3 Cfu | 2 Cfu |

Although hydrogels show exceptional wound healing properties, a drawback of the typical hydrogel is that microorganisms are often able to migrate through the matrix. Thus, if a wound is covered by hydrogel and one area of the wound becomes infected, the infectious organisms may be able to travel through the hydrogel and infect other regions. This possibility was tested by using a strip of hydrogel to bridge separated regions on a nutrient agar plate. Each agar plate was separated into two regions by removing a 2 cm strip of agar along a diameter of the plate. This gap was bridged by a 1.5 cm wide strip of hydrogel that overlapped onto the agar by about 5 mm at either end. One side of the plate was then inoculated with about 0.5 ml of culture and the plate was incubated to see if the microorganisms could cross the hydrogel “bridge.” The results in Table 14 show that silver hydrogel completely prevented migration

| Culture | Zone of Inoculation | Zone of Migration 5 |
|---|---|---|
| E. coil | Heavy Growth | No Growth |
| B. subtilis | Heavy Growth | No Growth |
| MRSA 1 | Heavy Growth | No Growth |
| Ps. Aeruginosa | Heavy Growth | No Growth |
| Hydrogel control | Heavy Growth | Growth |

From the results shown above a prototype gel formula was selected and the variations are suggested in the following examples.

### EXAMPLE A

For a 1 Kg batch of gel take components of Part A and Part B as given below:

| | | | |
|---|---|---|---|
| Part A | Inventive silver colloid 32 ppm ppm | 860 | g |
| | Distilled Water | 100 | g |
| | Carbapol | 6.8 | g |
| | Triethanolamine | 4.0 | g |
| Part B | Inventive silver colloid 32 ppm | 50 | g |
| | Propylene Glycol | 70 | g |
| | Triethanolamine | 6.0 | g |

First take required amount of distilled water and silver solution in a stirrer and start stirring. Slowly add in Carbapol (Noveon, USA). Stirring should be sufficiently vigorous to dispense the Carbopol and avoid formation of lumps. Temperature should be maintained between 60-70° C. during stirring.

Mix all ingredients of Part B in a beaker. Heat to 70° C. and add to Part A under vigorous stirring. Continue mixing and cool to room temperature. Check yield of batch. It should be approx. 1000 gm. The triethanolamine causes the Carbopol to gel.

### EXAMPLE B

Prepare all ingredients as in Example A including the addition of 1% collagen. This will give a gel with both antimicrobial as well as benefits of collagen which has scaffolding type of wound healing acceleration.

### EXAMPLE C

Prepare all ingredients as in Example A but including the addition of aloe vera (powder or solution) in the range of 1-5%. This will confer additional wound healing properties.

### EXAMPLE D

Prepare all ingredients as in Example A with the addition of 1-10% by weight Maltodextrin. This will provide a gel formulation which stimulates wound granulation.

Summary of Silver Hydrogel Results

It was possible to prepare Carbopol-based gels using inventive silver colloid solutions of 22 ppm and 32 ppm. The gels thus prepared have many advantages over their solution counterparts by virtue of their capacity to remain in place while retaining the properties of the parent silver solutions. The amorphous hydrogel nature of the medicament confers the advantage of moist wound healing acceleration and also limiting the severity of burns wound by limiting the thermal shock. Moreover, the active agent, colloidal silver solution has been tested on a cell line in an earlier study and found to be non-cytotoxic.

A thorough physico-chemical evaluation of the gel has been done with various batches and series of series of detailed methods were prepared to standardize and control product and processes during manufacture.

Microbiological studies were carried out in depth and show that the gel retains its bactericidal nature. Silver migration studies have been simulated and conclusively demonstrate that the gel can deliver silver over a period of time to the wound. The formulation design will also not allow microbe migration inside from outside and vice versa.

These tests demonstrate that hypothetical evaluation of the silver hydrogel on the basis of a publication (Journal of Wound Care Vol 12, No 8 September 2003) where alternative silver based dressings were evaluated with points given for:

1. Antimicrobial zone of inhibition;

2. Microbial challenge test;

3. Microbial transmission test; and

4. Silver content of dressings.

In the first test the silver hydrogel would be placed in group B and in all three remaining tests the silver hydrogel would score in group A giving it a total points of 20, on par with Calgitrol Ag and Acticot, commercial products which scored the highest in this evaluation.

The antibacterial and antiviral properties of the colloidal silver solution open up several significant uses for the silver hydrogel beyond a wound dressing. As demonstrated above the hydrogel is an ideal antibacterial hand scrub. In addition, the nonirritating character of the silver colloid and the hydrogel make the combination an ideal personal lubricant for male or female sexual use with or without condoms or diaphragms where the combination would combat bacteria, fungi (note the effectiveness on Candida albicans) and dangerous virus such as HIV and disinfect reusable barriers such as diaphragms. Since the hydrogel contains little if any oil, it has no harmful effects on condoms or diaphragms, unlike certain other personal lubricants.

HYDROGEL HAND CLEANSER (Note:Hydrogel and SILGEL refer to the same inventive product and are used interchangeably herein)

Clean hands have been reported to be the single most important factor in preventing the spread of dangerous germs and antibiotic resistance in health care settings. Accordingly, it was decided to check the efficacy of the hydrogel known as SILGEL as a hand hygiene product according to the guidelines of MMWR dated Oct. 25, 2002/VOL 51/No RR-16.

The Following Standard Operating Procedure was Utilized:

### Material Required (SOP):

Standard Suspension of Serratia marcescens (10⁸ cfu/ml), Tap water, Sterile Rubber gloves,

Sterile Sampling Solution, Sterile Tryptic Soya Agar, Sterile pippetes, Sterile test tubes.

### Method:

1. 5 ml of std. Suspension of Serratia marcescens_is applied to the hands and over the surfaces of the hands.
2. Spread 3 ml of test material over the hands and lower ⅓ rd of the fore arms.
3. Add 2 ml of tap water to the hands and lather them (refer FIG. 1)
4. Rinse hands and forearms under tap water for 30 seconds at RT.
5. Repeat the procedure from step 2 to step 4.
6. After 1^{st}, 3^{rd}, 7^{th} and 10^{th} washes, sterile rubber gloves used for sampling are placed on right and left hands.
7. 75 ml of sterile sampling solution is poured into the gloves.
8. All surfaces of the hands are massaged for one minute.
9. Samples are obtained aseptically for quantitative analysis by viable count method. using sterile Tryptic soya agar.
10. Spread plate technique is employed using original, 10⁻¹, 10⁻² and 10⁻³ as the dilutions.

Plates are incubated at 37° C. for 24 hrs.

### Materials and Methods

The procedures in the aforementioned SOP were utilized
Medium Used: St. Tryptic Soya Agar.
Cultures Used: 16 hr.old culture of Serratia marcescens (Approx.density is 10⁸ CFU/ml).
Incubation temperature: 37° C.
Incubation time: 24 hrs.
Products Evaluted: Silgel 22 and 32 ppm, Spitaderm, Liquid clean, Sterillium
Results: Results are presented in Tables 15a-15e , 16a-16e

### Left Hand

TABLE 15 a
Spitaderm (Appendix-II)

| No. of Washes | CFU/ml | | | |
|---|---|---|---|---|
| Dilution | Original | 10⁻¹ | 10⁻² | 10⁻³ |
| 1^{St} Wash | 10 | Nil | Nil | Nil |
| 3^{rd} Wash | Nil | Nil | Nil | Nil |
| 5^{th} Wash | Nil | Nil | Nil | Nil |
| 10^{th} Wash | Nil | Nil | Nil | Nil |

TABLE 15 b
Silgel 32 ppm

| No. of Washes | CFU/ml | | | |
|---|---|---|---|---|
| Dilution | Original | 10⁻¹ | 10⁻² | 10⁻³ |
| 1^{St} Wash | Nil | Nil | Nil | Nil |
| 3^{rd} Wash | Nil | Nil | Nil | Nil |
| 7^{th} Wash | Nil | Nil | Nil | Nil |
| 10^{th} Wash | Nil | Nil | Nil | Nil |

TABLE 15 c
Silgel 22 ppm

| No. of Washes | CFU/ml | | | |
|---|---|---|---|---|
| Dilution | Original | 10⁻¹ | 10⁻² | 10⁻³ |
| 1^{St} Wash | Nil | Nil | Nil | Nil |
| 3^{rd} Wash | Nil | Nil | Nil | Nil |
| 7^{th} Wash | Nil | Nil | Nil | Nil |
| 10^{th} Wash | Nil | Nil | Nil | Nil |

TABLE 15 d
Sterillium (Appendix-II)

| No. of Washes | CFU/ml | | | |
|---|---|---|---|---|
| Dilution | Original | 10⁻¹ | 10⁻² | 10⁻³ |
| 1^{St} Wash | 30 | 30 | Nil | Nil |
| 3^{rd} Wash | Nil | 10 | Nil | Nil |
| 7^{th} Wash | Nil | Nil | Nil | Nil |
| 10^{th} Wash | Nil | Nil | Nil | Nil |

TABLE 15 e
Liquid Clean (Appendix-II)

| No. of Washes | CFU/ml | | | |
|---|---|---|---|---|
| Dilution | Original | 10⁻¹ | 10⁻² | 10⁻³ |
| 1^{St} Wash | Nil | Nil | Nil | Nil |
| 3^{rd} Wash | Nil | Nil | Nil | Nil |
| 7^{th} Wash | Nil | Nil | Nil | Nil |
| 10^{th} Wash | Nil | Nil | Nil | Nil |

### Right Hand

TABLE 16 a
Spitaderm (Appendix-II)

| No. of Washes | CFU/ml | | | |
|---|---|---|---|---|
| Dilution | Original | 10⁻¹ | 10⁻² | 10⁻³ |
| 1^{St} Wash | Nil | Nil | Nil | Nil |
| 3^{rd} Wash | Nil | Nil | Nil | Nil |
| 5^{th} Wash | Nil | Nil | Nil | Nil |
| 10^{th} Wash | Nil | Nil | Nil | Nil |

TABLE 16 b
Silgel 32 ppm

| No. of Washes | CFU/ml | | | |
|---|---|---|---|---|
| Dilution | Original | 10⁻¹ | 10⁻² | 10⁻³ |
| 1^{St} Wash | Nil | Nil | Nil | Nil |
| 3^{rd} Wash | Nil | Nil | Nil | Nil |
| 7^{th} Wash | Nil | Nil | Nil | Nil |
| 10^{th} Wash | Nil | Nil | Nil | Nil |

TABLE 16 c
Silgel 22 ppm

| No. of Washes | CFU/ml | | | |
|---|---|---|---|---|
| Dilution | Original | 10⁻¹ | 10⁻² | 10⁻³ |
| 1^{St} Wash | Nil | Nil | Nil | Nil |
| 3^{rd} Wash | Nil | Nil | Nil | Nil |
| 7^{th} Wash | Nil | Nil | Nil | Nil |
| 10^{th} Wash | Nil | Nil | Nil | Nil |

TABLE 16 d
Sterillium (Appendia-II)

| No. of Washes | CFU/ml | | | |
|---|---|---|---|---|
| Dilution | Original | 10⁻¹ | 10⁻² | 10⁻³ |
| 1^{St} Wash | Nil | Nil | Nil | Nil |
| 3^{rd} Wash | Nil | Nil | Nil | Nil |
| 7^{th} Wash | Nil | Nil | Nil | Nil |
| 10^{th} Wash | Nil | Nil | Nil | Nil |

TABLE 16e
Liquid Clean (Appendix-II)

| No. of Washes | CFU/ml | | | |
|---|---|---|---|---|
| Dilution | Original | 10⁻¹ | 10⁻² | 10⁻³ |
| 1^{St} Wash | Nil | Nil | Nil | Nil |
| 3^{rd} Wash | Nil | Nil | Nil | Nil |
| 7^{th} Wash | Nil | Nil | Nil | Nil |
| 10^{th} Wash | Nil | Nil | Nil | Nil |

Conclusion: SILGEL 32 ppm and SILGEL 22 ppm fulfill the TFM (Tentative Final Monograph)
   criteria for efficiency as hand hygiene products which specifies the efficacy as 2-log₁₀ reduction of the indicator organism on each hand after 1^{st} use and a 3-log₁₀ reduction of indicator organism on each hand within 5 minutes of the 10^{th} use. Further SILGEL was more effective as a hand wash compared to Sterillium and Spitader. Finally being a ‘rub on’, SILGEL, as a hand cleanser, would be well tolerated because it eliminates the need for a sink and also does not make the user's hand dry or prone to irritation, but rather will tend to moisturize the area of usage.

The Hydrogel as a Wound Dressing Material

Introduction

Hydrogel dressings may be used as primary dressings (amorphous and impregnated gauzes) or as primary or secondary dressings (sheets) to manage partial and full-thickness wounds, deep wounds (amorphous, impregnated gauzes),wounds with necrosis or slough, minor burns and tissue damaged by radiation.

Nearly all hydrogels in the market today do not have an anti-microbial agent included. This is because antibiotics and antiseptics are potentially cytotoxic and often delay wound closure.

Since the inventive silver/water solution is non-cytotoxic it was decided to prepare a hydrogel using the inventive engineered silver nanoparticles.

Recently a specially prepared hydrogel has been introduced for the management of radiation induced dermatitis. These dressings have high specific heat to provide a cooling effect and will absorb at least three times in water, serum or blood.

Advantages
Are soothing and reduce pain
Rehydrate the wound bed
Facilitate autolytic debridement
Fill in bed space(amorphous, impregnated gauzes)
Provide minimal to moderate absorption
Applied and removed easily from the wound
Can be used when infection is present
Provides viewing of wound bed

Disadvantages
Are not usually recommended for wounds with heavy exudates
Some require secondary dressings
Dehydrate easily if not covered
Some may be difficult to secure
Some may cause maceration

Procedure

The hydrogel of the present invention was prepared in sheet form as a candidate wound dressing material. The sheet form of the hydrogel may be interchangeably referred to as SILDERM.

Results

SILDERM—Moisture Loss

Goal:
To determine the Moisture Loss ability of SILDERM.

Procedure:

Equipment Required:
Analytical Balance.

Material Required:
Plastic Tray

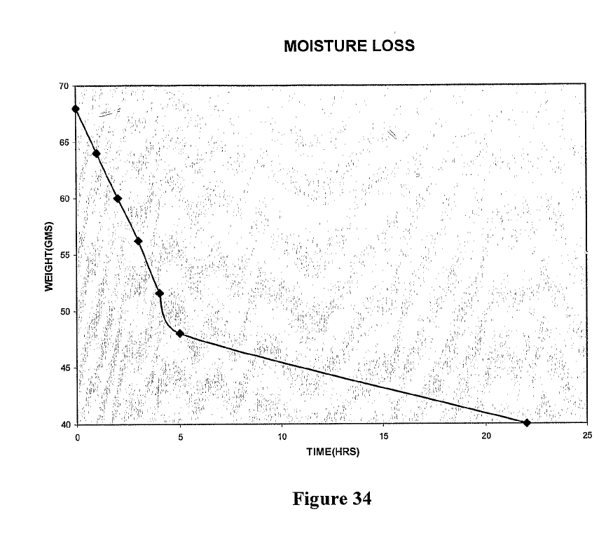

Method:
Determine the weight of an empty tray.
Place the SILDERM sheet onto the tray.
Determine the weight of tray+SILDERM sheet.
Note this reading as t=0 hrs.
Take the reading every 1 hr.
Take overnight reading.
Then plot a graph of time vs. moisture loss.
Determine percentage moisture loss.
Results: See Table 17 below and FIG. 34

TABLE 17
Moisture Losing Ability of SILDERM

| Time (hrs.) | Weight |
|---|---|
| 0 | 68 gm |
| 1 | 64 gm |
| 2 | 60 gm |
| 3 | 56 gm |
| 4 | 51 gm |
| 5 | 48 gm |
| 22 | 40 gm |

Conclusion: One can conclude that SILDERM sheet can lose 30% of its weight in moisture.

SILDERM—Moisture Uptake

Goal:
To determine the Moisture uptake ability of dehydrated SILDERM.

Procedure:

Equipment Required:
Analytical Balance.

Material Required:
Beaker

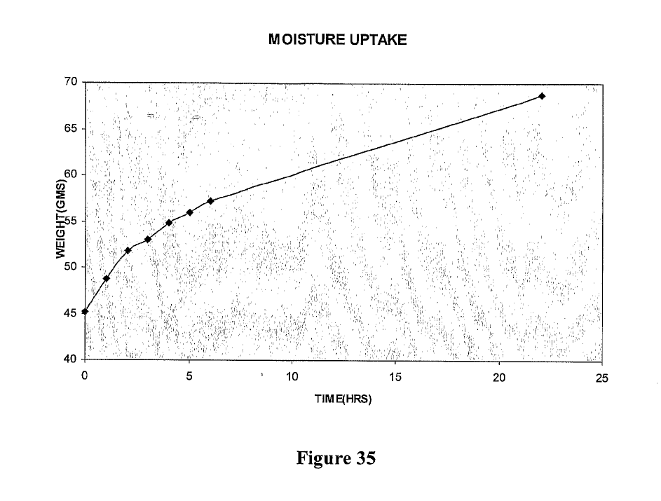

Method:
Determine weight of the SILDERM sheet in grams.
Note this reading as t=zero Hrs.
Fill the beaker with water.
Place the SILDERM sheet into the beaker immersing it completely.
At one hour intervals remove the sheet drip dry and determine its weight.
Take overnight reading.
Then plot a graph of time vs. Moisture uptake.
Calculate percentage of moisture uptake of the dehydrated gel.
Results: (See Table 18 below and FIG. 35)

TABLE 18

| Time (hrs.) | Weight |
|---|---|
| 0 | 45 gm |
| 1 | 48 gm |
| 2 | 51 gm |
| 3 | 53 gm |
| 4 | 54 gm |
| 5 | 56 gm |
| 6 | 57 gm |
| 22 | 68 gm |

Conclusion: Dehydrated SILDERM sheet can absorb up 52% of its weight in moisture.

SILDERM—SILVER RELEASE

Goal:
To determine the sustained release of silver nanoparticles from SILDERM

Principle:
Hydrogel dressing sheets are normally placed on a wound for 48-72 hrs. In this situation it would be desirable to determine antimicrobial activity of the dressing over this time period with respect to silver release.

Equipment Required:
Incubator, Laminar Flow

Material Required:
Sterile Nutrient Agar Plates, Sterile Cotton Swabs, Micropipette (Capacity 100 μl-1000 μl) 16 hr. old culture of Pseudomonas aeruginosa (wild type)

Method:
Cut a 4 cm×3 cm piece of SILDERM.
Place the Silderm on a nutrient agar plate swabbed with Ps. aeruginosa (wild type).
Incubate at 37° C. for about 18 hrs.
Check the zone of inhibition vertically and horizontally.
Then place the same SILDERM piece on a freshly swabbed nutrient agar plate with Ps. aeruginosa.
Incubate as above.

Repeat this procedure for minimum of 7 days.
Results: At the time of printing SILDERM showed inhibitory activity for 3 transfers as given in Table 19 below.

TABLE 19
SILDERM Challenge Test

| Transfer No | Vertical Inhibition | Horizontal Inhibition |
|---|---|---|
| Transfer 1 | 52 mm | 35 mm |
| Transfer 2 | 53 mm | 35 mm |
| Transfer 3 | 51 mm | 34 mm |

Conclusion: SILDERM hydrogel was able to exert sustained antimicrobial activity over 3 challenges of fresh inoculum every 24 hrs. Further testing is in progress.

The media composition for the above-discussed embodiments was as follows:

### Nutrient Agar:

| Peptone | 10.0 gm |
|---|---|
| Sodium chloride | 5.0 gm |
| Meat extract | 3.0 gm |
| Distilled water | 900 ml |
| Agar | 2.5 gm |
| pH | 7.2 ± 0.2 |

Further, it will be possible to develop formulations of SILDERM with the following:

Collagen—
Collagen, the most abundant protein in the body, is fibrous and insoluble and is produced by fibroblasts. Its fibers are found in connective tissues, including skin, bone, ligaments, and cartilage. During wound healing, collagen encourages the deposition and organization of newly formed collagen fibers and granulation tissues in the wound bed. It also stimulates new tissue development and wound debridement, creating an environment conducive to healing.

Maltodextrin—
Maltodextrin is a wound healing promoter that hastens healing by macrophage activation and attraction, thereby reducing infection and increasing granulation.

Platelet Derived Growth Factors (PDGF)—
PDGF promote the chemotactic recruitment and proliferation of cells involved in wound repair and enhancing formation of granulation tissue. It is mainly used to treat lower extremity diabetic neuropathic ulcers.

DiSodium EDTA as an ADDITIVE

DiSodium EDTA has been known to increase the antibacterial effect of various compounds, both natural and synthetic, by a mechanism that has been presumed to result in increasing bacterial cell wall permeability, thus facilitating entry of antibacterial compounds.

The metal chelator and bacterial outer membrane permeabilizer, ethylene diamine tetra acetic acid (EDTA), has been shown to enhance the activities of various anti-microbial agents against Pseudomonas aeruginosa. The addition of a subinhibitory concentration of EDTA markedly reduced the MICs of cefprozil against E. coli and Serratia marcescens.

It has been reported that that imipenem, ceftazidime and cefepime plus 150 mcg of EDTA could increase the mean inhibition zone diameter for P. aeruginosa. A study reported that Ethylenediaminetetraacctic acid (EDTA) influenced the susceptibility of P. aeruginosa. EDTA when used in conjunction with AgNO₃ enhanced the antibacterial action of the latter significantly, so that strains of Klesbiella pneumoniae and Staphylococcus aureus resistant to 70 microgram/ml of AgNo3 were observed to become sensitive to 10 microgram/ml of this compound.

A specific composition and set of tests was designed to determine if silver/water compositions of the present invention would function favorably with Disodium EDTA. Specifically, DiSodium EDTA was procured form West Coast Laboratories in Mumbai, India. DiSodium EDTA is also known as Na₂EDTA (DiSodium Ethylenediaminetetra Acetic Acid) and has a formula: (CH₂N(CH₂COOH)CH₂ COONa)₂ 2H₂O and has a molecular weight of 372.24.

The media used for this test was: Nutrient Agar: (HiMedia) 1000 ml; B.No. 1G115 exp. August 2006; Peptic digest of animal tissue 50.00 g; Yeast extract 1.50 g; Beef extract 1.50 g; Sodium Chloride 5.00 g; Agar Agar type-I 25 g; pH T4+/−0.2.

### Microbial Strains

The 32 ppm silver/water composition alone, the 22 ppm silver/water composition alone, and the 32 ppm silver/water composition, as well as the 22 ppm silver water composition, were added to the

Na₂EDTA, and were each tested against a panel of microorganisms, including:
Escherichia coli (Multiple Drug-Resistant Strain) from stool sample;
Pseudomonas aeurginosa (Multiple Drug-Resistant Strain) from sputum; and
Methicillin Resistant Staphylococcus aureus (Multiple Drug-Resistant Strain) from pus from the lumbar region.
The above mentioned MDR strains were obtained from P.D. Hinduja Hospital (MUMBAI, India).
Shigella flexneri (lab strain)
Salmonella typhi (lab strain)
Bacterial strains were cultured for 24 hrs. at 37° C. on Nutrient Agar (pH 7.4).

Dilutions for the 32 ppm and 22 ppm added to the Na₂EDTA were prepared in sterile distilled water. Each microorganism was suspended in sterile saline and diluted at 10⁶ colony forming units (cfu/ml). They were swabbed onto the surface of Nutrient agar (pH 7.4) using sterile cotton swabs. The wells (10 mm in diameter) were punched from the agar and 0.1 ml of the respective dilutions were delivered into them. After incubation for about 24 hrs. at 37° C., all plates were examined for any zones of growth inhibition and the diameters were measured in mm using a zone reader (Hi Media). Results are given in Table 20.

### Results and Discussion

TABLE 20
Silver/Water + Na₂EDTA.

| System | E. coli | MRSA | C. albicans |
|---|---|---|---|
| Silver/Water 32 ppm − | 21 mm | 24 mm | 27 mm |
| Ctrl | | | |
| Silver/Water 32 ppm + | 22 mm | 29 mm | 40 mm |
| 0.5% Na₂ EDTA | | | |
| Silver/Water 22 ppm + | 20 mm | 23 mm | 29 mm |
| Silver/Water 22 ppm + | 22 mm | 31 mm | >40 mm |
| 0.5% Na₂ EDTA | | | |

DiSodium EDTA at 0.5 ppm is definitely enhancing the potency of silver/water compositions of the present invention at both 22 and 32 ppm concentration levels.

Silver EDTA as a Stand Alone Antibacterial

A specific composition and set of tests was designed to determine if silver chelates such as silver EDTA (or AgEDTA) possess antibacterial qualities. Specifically, commercially available silver EDTA compositions were procured from AKZO Nobel and Alpha Chemicals.

Equipment Required:
Incubator, Laminar Flow

Material Required:
Sterile Nutrient Agar Plates, Sterile Cotton Swabs, Micropipette (Capacity 100 μl-1000 μl)
   16 hr. old culture of following strains (Appr. density is 10⁸ CFU/ml), Escherichia coli (wild type), Escherichia coil (MDR), Pseudomonas aeruginosa (wild type), Pseudomonas aeruginosa (MDR), Staphylococcus aureus ATCC 6538P, Methicillin Resistant Staphylococcus aureus.

Method:
Swab the 16 hr. old culture of given test organisms on a sterile nutrient agar plate.
Allow the plates to sit for 15 minutes for absorption.
After 15 minutes aseptically bore wells into the agar surface with the help of a 10 mm cork borer.
Dispense 100 μl of the appropriate sample dilution in the wells. Maintain for 15 minutes for pre diffusion.
Incubate the plates at about 37° C. for about 24 hrs. and observe results.
Measure the zone of inhibition in mm using HiMedia zone reader.

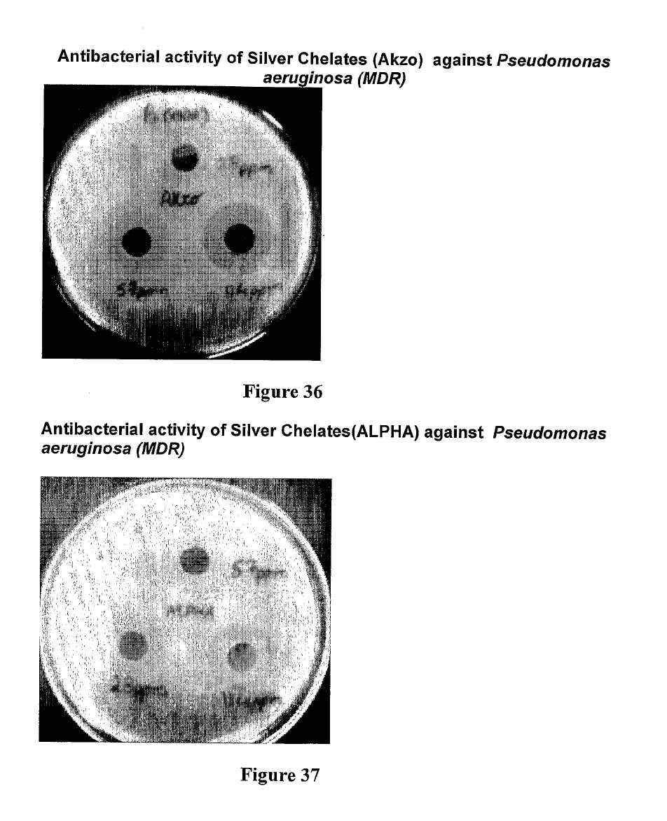

Results: See Table 21 below and FIGS. 36 and 37.

TABLE 21
Comparative evaluation of Silver chelates

Zone of Inhibition

| Organism | Conc. | | AKZO | ALPHA |
|---|---|---|---|---|
| E. coli | 28 | ppm | 20 mm | 22 mm |
| (wild type) | 57 | ppm | 22 mm | 24 mm |
| | 114 | ppm | 22 mm | 25 mm |
| E. coli (MDR) | 28 | ppm | 20 mm | 19 mm |
| | 57 | ppm | 21 mm | 21 mm |
| | 114 | ppm | 23 mm | 22 mm |
| Ps. | 28 | ppm | 21 mm | 20 mm |
| aeruginosa | 57 | ppm | 27 mm | 24 mm |
| (wild type) | 114 | ppm | 28 mm | 27 mm |
| Ps. | 28 | ppm | 15 mm | 17 mm |
| aeruginosa | 57 | ppm | 21 mm | 20 mm |
| (MDR) | 114 | ppm | 25 mm | 22 mm |
| S. aureus | 28 | ppm | 16 mm | 15 mm |
| (wild type) | 57 | ppm | 19 mm | 18 mm |
| | 114 | ppm | 22 mm | 21 mm |
| MRSA | 28 | ppm | 19 mm | 20 mm |
| | 57 | ppm | 21 mm | 22 mm |
| | 114 | ppm | 26 mm | 24 mm |

Conclusion: Silver chelates such as silver EDTA possess antibacterial efficacy.

Antibiotics Combination Therapy

When first discovered, antibiotics were touted as a miracle cure and they literally were. Infections that were fatal before the turn of the century were tamed to mere inconveniences during this century. But medicine has come almost full circle. Misuse, over prescriptions and/or abuse of antibiotics has allowed resistant strains of bacteria to develop and once again bacteria strains threaten health and life.

Some of the other factors that contribute to the development of resistance by bacteria are the use of antibiotics for agricultural purposes and as food supplements in agriculture (e.g., for poultry, cattle, pork, etc.). Over prescribing of antibiotics is thought by many to be rampant in the agricultural industry in the United States and is rampant in many foreign countries. Therapy with antibiotics in agriculture often are started even before the culture specimen is sent to the laboratory. Avian influenza (e.g. H5N1 or “HPAI”) has become very antibiotic resistant due to the heavy usage of antibiotics by Asian poultry farmers. Patients also have easy access to antibiotics over the counter. Improper dosages and incomplete treatment duration also contribute to the emergence of drug resistant strains. There are important clinical ramifications to the problem of resistance. Resistance of pathogenic bacteria to antibiotics has had a severe effect on the treatment of infectious diseases. Many drugs, such as penicillin, which was believed to be a wonder drug had great potential for effective control when it was first introduced, only to have bacteria, adapt to them and greatly reduce their applicability.

The problem of antibiotic resistance is today a global problem. Some common and highly pathogenic bacteria such as Staphylococcus aureus particularly the strains found in hospitals, are now known to be resistant to all but vancomycin, and are soon expected to be vancomycin-resistant too. MRSA (Methicillin Resistant Staphylococcus aureus) and VRE (Vancomycin Resistant Enterococci) are a cause of severe nosocomial infections and often hospital wards are closed, and even destroyed, when detected.

With this problem it is imperative to the search for alternative such as, either new antibiotics to take the place of the old ones or make efficient use of the existing antibiotics. Also the growing threat of multi-drug resistant bacteria is a good reason to consider silver/water compositions according to the present invention.

One of the ways to deal with the ever increasing resistance of bacteria to antibiotics involves the use of combination therapy, which uses of two or more different antibiotics with different modes of action. Various in-vitro methods are available for measurement of the synergic effects of combinations of antibiotics, but the results may exhibit discrepancies when different tests are used, also this does not totally rule out the development of resistance to them.

Aims and Objectives

The present study was conducted with the following aims and objectives:
1. To determine Multi Drug Resistance (MDR) pattern of the clinical isolates.
2. To determine the sensitivity of the clinical isolates to silver/water solutions of the present invention.
3. To determine the antibiotic combination (synergy) by disc approximation test.
4. To determine the minimum inhibitory concentration (MIC) of the antibiotics and silver/water compositions of the present invention.
5. To study the synergistic action between antibiotics and silver/water solutions of the present invention by checkerboard assay.

Materials and Methods

### Collection of Clinical Isolates

The following Multi Drug Resistant clinical isolates were collected from P.D. Hinduja Hospital,

Cadell road, Mahim, Mumbai-400016, India.
Escherichia coli. (isolated from stool)
Pseudomonas aeruginosa.(isolated from sputum)
Methicillin Resistant Staphylococcus Aureus. (MRSA-isolated from pus from lumbar
region)

Media, Solutions and Antibiotic Discs:

Media:
Nutrient broth.
Nutrient Agar.
Muller and Hinton Agar.

Solutions:
Antibiotic solutions.
Silver/Water solution (22 ppm).

The media composition and the solutions used for the various experiments in the study are listed in Table 26 (later herein).

Readily available antibiotic disc of appropriate concentration were used. The disc content for each antibiotic is listed in Table 27 (later herein).

### Inoculum Preparation:

A loopful of pure growth of the culture was inoculated into Nutrient Broth and incubated overnight at about 37° C. 500 mcl of the overnight culture was transferred to 5 ml of fresh Nutrient Broth and incubated for 4-6 hrs at about 37° C. The culture density is adjusted to about 10⁵-10⁶ cfu/ml.

Antibiotic sensitivity test—Kirby Bauer method:

In this method antibiotic impregnated discs are placed on the agar plate, previously inoculated with the bacterial suspension. The antibiotic diffuses out in the surrounding medium. There is alogarithmic reduction in the antibiotic concentration as the distance from the disc increases. A clear zone around the disc indicates the susceptibility of the organism to the antibiotic. Clear zones are measured in millimeters and compared with a standard NCCLS chart.

Method:
1. Sterile cotton swabs were dipped in the above inoculum broth tubes and used to surface spread on M.H.Agar plates to obtain confluent growth.
2. After allowing the inoculum to absorb in the medium, antibiotic discs were placed on the surface spread plates with the help of sterile forceps.
3. The plates were incubated at about 37° C. for about 24 hours.
4. A clearing around the disc indicates sensitivity of the organism. The zone diameters are recorded and interpreted according to the standard charts provided by NCCLS. (Refer to Table 27) (Koneman 5^{th} ed. 1997).

Determination of the sensitivity of the isolates to 10 ppm-agar diffusion method: This was determined by the well assay method in which the isolated is bulk seeded in the agar medium and the 10 ppm silver/water solution is added in the wells (10 mm) punched in the solid inoculated medium. The zone size of inhibition is then noted.

Method:
1. To a molten butt of Muller and Hinton agar 20 ml, 0.5 ml of the inoculum is added and poured in a petri plate and allowed to solidify.
2. Wells are punched in to the agar layer.
3. Different concentrations of silver/water compositions are then added to each of the wells.
4. The plates were incubated at about 37° C. for about 24 hours.
5. Note the size of zones of inhibition.

Determination of antibiotic combination—by disc diffusion test.

This is a simple, qualitative, test for testing the interaction between the clinical isolate and combination of antibiotic. In this test the antibiotic discs are placed on agar plate inoculated by Kirby-Bauer technique. The discs should be separated by a distance that is equal to or slightly greater than the average of the diameters of inhibition produced by each disc alone. The shape of the inhibitory zone obtained will indicate the kind of interaction between the clinical isolate and combination of antibiotic.

Method:
1. Sterile cotton swabs were soaked in the above inoculum broth tubes and used to surface spread on M.H.Agar plates to obtain confluent growth.
2. After allowing the inoculum to absorb in the medium, two antibiotic discs (the combination to be studied) were placed on the surface spread plates with the help of sterile forceps, at a distance equal to or slightly greater than the sum of the diameters of inhibition produced by each disc alone.
3. The plates were incubated at about 37° C. for about 24 hours.
4. Shape of the inhibitory zones would indicate the type of interaction, i.e. synergy, antagonism or indifference.

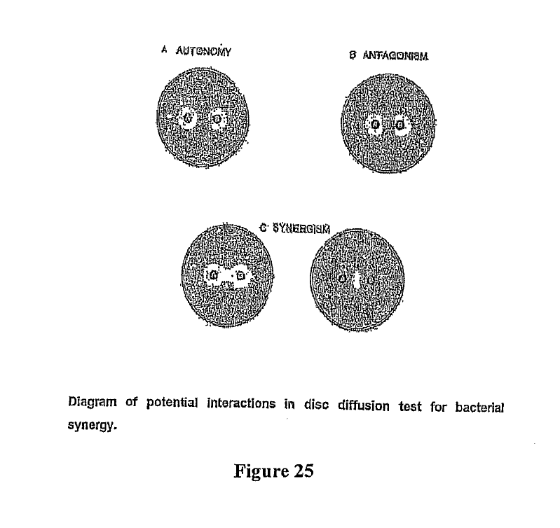

FIG. 25 is a diagram which shows the potential interactions in a disc diffusion test for bacterial synergy.

Specifically, Part A demonstrates additive or indifference effects; each antibiotic produces a zone of inhibition that is not affected by the adjacent one; Part B demonstrates antagonistic effects in which the inhibitory zones of each antibiotic are diminished in presence of another antibiotic; and Part C demonstrates two possible manifestations of synergistic interactions. On the left an enlarged inhibitory zone occurs where the two antibiotics meet. On the right neither antibiotic is inhibitory in its own right, but bacterial growth is inhibited where the two antibiotics diffuse together.

Determination of the minimum inhibitory concentration (MIC) of the antimicrobial agents. This is a macrodilution broth susceptibility test. Serial dilutions of the antimicrobial agent are prepared in broth to which standardized bacterial suspension is added. At the end of the incubation period the tubes are observed visually for growth. The lowest concentration of the antimicrobial agent that inhibits visible growth is taken as the MIC.

Antibiotics used:
Amikacin: Mikacin inj. (250 mg) Aristo labs, Mumbai India. Batch no.02D054, mfd April 2004.
Cefoperazone: Magnamycin inj. (250 mg) Pfizer ltd, Mumbai, Ind Batch no. 32035153A, mfd March 2003
Ciprofloxacin: Cifran (200 mg/ml) Ranbaxy Labs, Jaipur, India Batch no. 9042601, mfd March 2004.

Method:
1. A quantity of antimicrobial agent is serially diluted in a suitable range.
2. A tube free of serves antimicrobial agent serves as growth control.
3. Each of the tubes is then inoculated with a standardized bacterial suspension and incubated at about 37° C. for about 24 hours.
4. At the end of the incubation period, the tubes are visually examined for turbidity.

Turbidity indicates that the bacterial growth has not been inhibited by the concentration of the antimicrobial agent contained in the medium.
5. MIC is the lowest concentration of the antimicrobial agent that inhibits visible growth.

Study of the synergistic action by checkerboard assay.

Checkerboard assay is a method used, when multiple antibiotics and/or multiple dilutions are to be tested. Serial two fold dilutions are selected so that concentrations from one sixteenth to at least double the MIC are included. Drug A is serially diluted along the ordinate, while drug B is serially diluted along the abscissa. The resulting checkerboard yields every combination of the two antibiotics, from a tube that contains the highest concentration of each at the opposite corner.

Arrangement of drug dilutions in checkerboard assay.

First row and column of tubes with only one drug served to confirm individual MIC values of test isolates.

One tube without antibiotic is the positive control.
1. In a final volume of 5 ml in each tube antimicrobial drugs diluted in broth are to be added from appropriate stock solutions.
2. Add 0.1 ml of the culture suspension.
3. Incubate at 37° C. for 24 hrs.
4. Results are depicted by drawing an isobologram obtained by joining points that represent all combinations with same effect, including equally effective concentration of antibiotic used alone.

Calculations:

Elion et al (1954) described a method for quantifying MIC results obtained in trems of

Fractional inhibitory concentration (FIC) index, defined as sum of FIC values of two drugs in combination.

FIC index=FIC of drug A+FIC of drug B.

$$FIC \text{of drug} A = \frac{MIC \text{of drug} A \text{in combination with drug} B .}{MIC \text{of drug} A .}$$

An index of less than 0.5 is considered evidence of synergism; an index of greater than 2.0 is evidence of antagonism.
(Koneman 5^{th} ed. 1997)

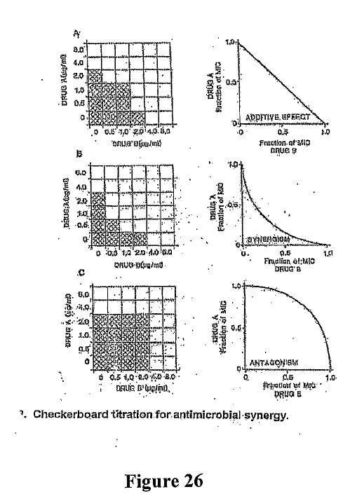

FIG. 26 shows a Checkerboard titration for antimicrobial synergy.

Each square represents a tube. Increasing concentrations of antibiotic A are distributed along the horizontal axis, and those of antibiotic B along the vertical axis. The hatched squares indicate bacterial growth. In panel A the antibiotics demonstrate additive effect; the isobologram on the right is a straight line. Panel B represents synergism where the isobologram is a concave curve. Panel C shows antagonistic results with a convex curve.

Determination of antibiotic sensitivity pattern by Kirby-Bauer method.

TABLE 22
Antibiograms of the isolates used for the study. (zones in mm.)

| | Zone size (mm) | | |
|---|---|---|---|
| | Organisms | | |
| Antibiotics | E.c | Ps. | MRSA |
| Amikacin | 20 | 9 | 22 |
| Ciprofloxacin | — | — | 20 |
| Kanamycin | 14 | — | — |
| Gentamycin | 19 | — | — |
| Tetracycline | — | — | 27 |
| Nalidixic acid | — | — | — |
| Cefoperazone | — | 14 | 23 |
| Ceftazidime | 10 | 16 | — |
| Chloramphenicol | — | — | — |

| | Zone size in mm | | |
|---|---|---|---|
| ASAP conc | Organisms | | |
| ppm | E. coli | Pseudomonas | MRSA |
| 32 | 16 | 16 | 13 |
| 16 | 15 | 14 | 11 |
| 8 | 11 | 11 | — |
| 4 | — | — | — |
| 2 | — | — | — |

Key: — No inhibition.
Determination of sensitivity of the clinical isolates to ASAP-agar diffusion method.

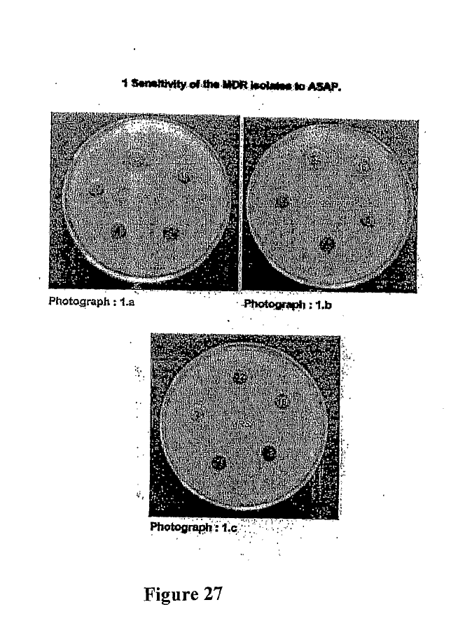

See FIG. 27 for photographs.

Determination of antibiotic combination by disc approximation test.

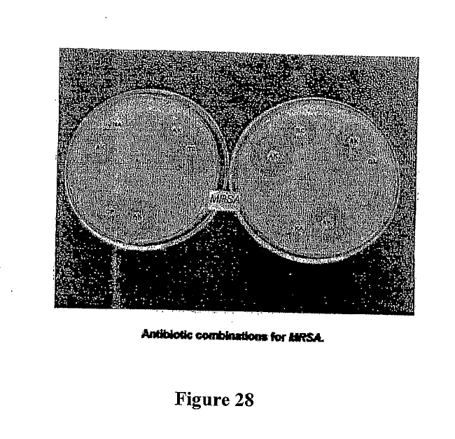

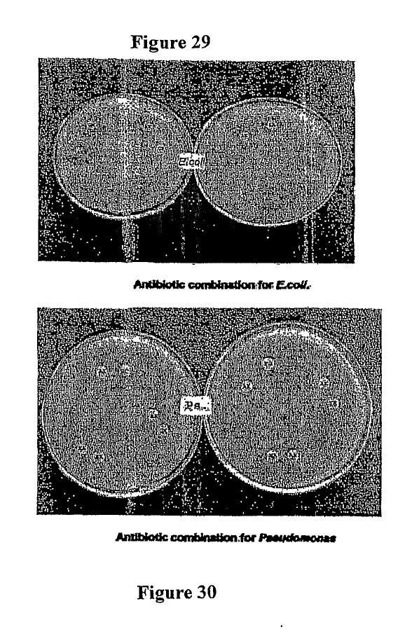

Of the various antibiotic combinations checked for synergistic or additive effect on the isolates, inhibitory zones indicative of possible synergy was observed only in case of MRSA for the combination of Amikacin with Cefoperazone and Amikacin with Tetracycline. (See FIG. 28). No inhibitory zones suggestive of synergic combination were observed in case of the other two isolates i.e. E.coli and Pseudomonas. (See FIGS. 29 and 30).

Determination of the Minimum inhibitory concentration of antibiotics.

The MIC of the antibiotics that showed inhibitory zones suggestive of possible synergy was determined.

TABLE 23

MIC of Amikacin
Stock: 125 mcg/ml
Diluent: Nutrient Broth
Culture: MRSA
The MIC of Amikacin for MRSA was found to be 0.8 mcg/ml.
MIC of Cefoperazone
Stock: 100 mcg/ml
Diluent: Nutrient broth
Culture: MRSA

| | Conc. | |
|---|---|---|
| Tube no. | mcg/ml | Growth |
| 1 | 0.2 | + |
| 2 | 0.4 | + |
| 3 | 0.6 | + |
| 4 | 0.8 | − |
| 5 | 1 | − |
| 6 | 2 | − |
| 7 | 3 | − |
| 8 | 4 | − |
| 9 | 5 | − |
| 10 | +ve | + |
| 11 | −ve | − |

The MIC of Cefoperazone for MRSA was found to be 10 mcg/ml.
MIC of Silver/Water
Stock: 20 ppm of silver/water solution

| | Conc. | |
|---|---|---|
| Tube no. | (ppm) | Growth |
| 1 | 5 | + |
| 2 | 10 | − |
| 3 | 15 | − |
| 4 | 20 | − |
| 5 | 25 | − |
| 6 | 30 | − |
| 7 | 35 | − |
| 8 | 40 | − |
| 9 | 45 | − |
| 10 | 50 | − |
| 11 | + ve | + |
| 12 | −ve | − |

Key:
+: Growth
−: No growth.
Diluent: Nutrient Broth
Culture: MRSA

TABLE 23a

| | Conc. | |
|---|---|---|
| Tube no. | (ppm) | Growth |
| 1 | 1 | + |
| 2 | 2 | + |
| 3 | 3 | + |
| 4 | 4 | + |
| 5 | 5 | + |
| 6 | 6 | + |
| 7 | 7 | + |
| 8 | 8 | − |
| 9 | 9 | − |
| 10 | 10 | − |
| 11 | +ve | + |
| 12 | −ve | − |

Key:
+: growth.
−: No growth.
The MIC of Silver/Water for MRSA was found to be 8 ppm.
MIC of Silver/Water
Stock: 20 ppm
Diluent: Nutrient Broth
Culture: E. coli

TABLE 24

| | Conc | |
|---|---|---|
| Tube no. | (ppm) | Growth |
| 1 | 1 | + |
| 2 | 2 | + |
| 3 | 3 | − |
| 4 | 4 | − |
| 5 | 5 | − |
| 6 | 6 | − |
| 7 | 7 | − |
| 8 | 8 | − |
| 9 | 9 | − |
| 10 | +ve | + |
| 11 | −ve | − |

Key:
+: Growth.
−: No growth.
The MIC of Silver/Water for E. coli was found to be 3 ppm.
MIC of Silver/Water
Stock: 20 ppm silver/water
Diluent: Nutrient Broth
Culture: Pseudomonas

TABLE 25

| | Conc | |
|---|---|---|
| Tube no. | (ppm) | Growth |
| 1 | 1 | + |
| 2 | 2 | + |
| 3 | 3 | − |
| 4 | 4 | − |
| 5 | 5 | − |
| 6 | 6 | − |
| 7 | 7 | − |
| 8 | 8 | − |
| 9 | 9 | − |
| 10 | +ve | + |
| 11 | −ve | − |

Key:
+: growth.
−: No growth.
The MIC of Silver/Water for Pseudomonas was found to be 3 ppm

Study of the synergistic action by checkerboard assay.

I. Combination of Amikacin and Silver/Water.

MIC of Amikacin=0.8 mcg/ml.

MIC of Silver/water=8 ppm.

Culture: MRSA

The synergic concentration for MRSA was found to be 0.05 mcg/ml of amikacin and 1 ppm of silver/water of the present invention.

Calculation of the AC index:

$$\begin{matrix}FIC of Amikacin = \frac{MIC of amikacin in combination}{MIC of Amikacin alone .} \\ = 0.05 / 0.8 \\ = 0.0625 .\end{matrix}$$

$$\begin{matrix}FIC of ASAP = \frac{MIC of \text{Silver/Water} in combination}{MIC \text{of Silver/Water alone.}} \\ = 1 \text{/} 8 \\ = 0.125 .\end{matrix}$$

$$\begin{matrix}FIC index = FIC of Amikacin + FIC \text{of Silver/Water} \\ = 0.0625 + 0.125 \\ = 0.1875 .\end{matrix}$$

FIC index is indicative of synergy between Amikacin and Silver/Water

II. Combination of Cefoperazone and Silver/Water.

MIC of Cefoperazone=10 mcg/ml.

MIC of Silver/water=8 ppm.

Culture: MRSA

The synergic concentration for MRSA was found to be 0.625 mcg/ml of Cefoperazone and 1 ppm of Silver/water

Calculation of the FIC index:

$$\begin{matrix}FIC of Cefoperazone = \frac{MIC of Cefoperazone in combination}{MIC of Cefoperazone alone .} \\ = 0.625 / 10 \\ = 0.0625 .\end{matrix}$$

$$\begin{matrix}FIC of ASAP = \frac{MIC of \text{Silver/Water} in combination}{MIC \text{of Silver/Water alone.}} \\ = 1 \text{/} 8 \\ = 0.125 .\end{matrix}$$

$$\begin{matrix}FIC index = FIC of Amikacin + FIC \text{of Silver/Water} \\ = 0.0625 + 0.125 \\ = 0.1875 .\end{matrix}$$

FIC index is indicative of synergy between Cefoperazone and Silver/Water.

III. Combination of Cefoperazone and Amikacin.

MIC of Cefoperazone=10 mcg/ml.

MIC of Amikacin=8 ppm.

Culture: MRSA

The additive concentration of Cefoperazone was found to be 1.25 of Amikacin was found to be 0.4.

Calculation of the FIC index:

$$\begin{matrix}FIC of Cefoperazone = \frac{MIC of Cefoperazone in combination}{MIC of Cefoperazone alone .} \\ = 1.25 / 10 \\ = 0.125\end{matrix}$$

$$\begin{matrix}FIC of Amikacin = \frac{MIC of Amikacin in combination}{MIC \text{of Amikacin alone.}} \\ = 0.4 \text{/} 0.8 \\ = 0.5\end{matrix}$$

$$\begin{matrix}FIC index = MIC of Amikacin + FIC \text{of Cefoperazone} \\ = 0.125 + 0.5 \\ = 0.625\end{matrix}$$

FIC index is indicative of addition between Cefoperazone and Silver/Water.

Discussion

In this Example, of the three clinical isolates collected from P. D. Hinduja Hospital, Mumbai, India, gram-negative isolates showed resistance to older antibiotics such as ampicillin, tetracycline, kanamycin, and older quinolones like nalidixic acid as well as the third generation cephalosporins—ceftazidime and cefoperazone. The clinical isolates of Pseudomonas used for the study was also resistant to the recent ciprofloxacin and to the semi-synthetic aminoglycoside, amikacin. The gram-positive isolate of MRSA was also resistant to the older antibiotics and also to the third generation cephalosporins like ceftazidime.

The study of their sensitivity to silver/water compositions of the present invention showed that the gram-negative isolates were readily sensitive to about 3 ppm Silver/Water solutions and MRSA isolate was found to be inhibited by 8 ppm Silver/Water solutions as determined by the agar diffusion and macrodilution broth method.

The interaction of the two antibiotics in combination with the isolates was determined by the disc diffusion method, which revealed synergic results between cefopoerazone and amikacin against MRSA. A checkerboard assay was carried out to confirm this. No addition or synergy between antibiotics was observed for the gram-negative isolates by disc diffusion test.

Checkerboard assay was performed and the FIC index of the two antibiotics was found to be 0.625 thus indicating addition and not synergy for the combination of amikacin and cefoperazone.

Checkerboard assay was also performed to study the combination of Silver/Water solutions with the amikacin and also with cefoperazone. The results showed that in presence of the inventive silver/water compositions, the effective concentration of the antibiotic was reduced by about four fold. The FIC index of these combinations was found to be 0.1875 in each case indicating synergy for the combination of silver/water with amikacin and silver/water with cefoperazone.

The results of the study indicate that in the above clinical MDR isolates the antibiotic dose could be greatly reduced in presence of silver/water, which was not observed to be the case in antibiotic combination.

These results show that the inventive silver/water compositions will have an important role to play in combination antibiotic therapy especially against Multi Drug Resistant strains.

TABLE 26

| 1. Nutrient Broth: | | |
|---|---|---|
| Peptone | 10.0 | gm |
| Sodium chloride | 5.0 | gm |
| Meat extract | 3.0 | gm |
| Dextrose | 5.0 | gm |
| Phenol red (indicator) | 0.001% | |
| Distilled water | 900 | ml |
| 2. Nutrient Agar: | | |
| Peptone | 10.0 | gm |
| Sodium chloride | 5.0 | gm |
| Meat extract | 3.0 | gm |
| Distilled water | 900 | ml |
| Agar | 2.0% | |
| pH | 7.2 | |
| 3. Muller and Hinton Agar | | |
| Casein acid hydrolysate | 29.0 | gm |
| Beef starch | 10.0 | gm |
| Potato starch | 2.5 | gm |
| Agar | 1.2% | |
| Distilled water | 1000 | ml |

| | |
|---|---|
| PH | 7.6 |

TABLE 27
Zone diameter interpretation (NCCLS Document, 1988)

| | Disc | | | |
|---|---|---|---|---|
| | conc | Zone diameter in mm | | |
| Antibiotics | (mcg) | Resisitant | Intermidiate | sensitive |
| Amikacin (AK) | 30 | ≦14 | 15-16 | ≧17 |
| Ciprofloxacin (RC) | 5 | ≦15 | 16-20 | ≧21 |
| Kanamycin (KA) | 30 | ≦13 | 14-17 | ≧18 |
| Gentamycin (GM) | 10 | ≦12 | 13-14 | ≧15 |
| Tetracycline (TE) | 30 | ≦14 | 15-18 | ≧19 |
| Nalidixic acid (NA) | 30 | ≦14 | 14-18 | ≧19 |
| Cefoperazone (CP) | 75 | ≦15 | 16-20 | ≧21 |
| Ceftazidime (FG) | 30 | ≦14 | 15-17 | ≧18 |
| Chloramphenicol (CH) | 30 | ≦12 | 13-17 | ≧18 |

Combination of Gentamycin and Silver/Water Compositions as a Wound Dusting Powder

Introduction

Wound dusting powders are formulations used for the prevention or treatment of surface bacterial infections of wounds, burns skin ulcers or abcesses after incision.

Wound powders are normally broad spectrum antibiotics/antiseptic preparations. Use of such powders does not exclude concomitant therapy with antibiotics where appropriate.

Most wound products available in the market today are based on Povidone—Iodine. Povidone—Iodine is highly cytotoxic in open wounds and has specifically been contraindicated in diabetic wounds. Further, iodine sublimes and has to be reapplied about every 6-8 hrs.

Another potential field of application is in the Veterinary area. Pets often incur cuts, abrasions, and wounds either due to scratching to eliminate parasites as well as encounters with other animals. A mild but broad spectrum antimicrobial would be helpful in this application.

It was decided to formulate a wound dusting powder consisting of a slow release preparation combining Gentamycin and the engineered silver nanoparticles of the present invention. A talc-based preparation containing about 200 ppm silver nanoparticles and about 100 ppm Gentamycin is referred to herein as SILDUST.

Results

SILDUST—Sensitivity

Goal: To determine Sensitivity of SILDUST and its constituents against microorganisms.

Procedure:

Equipment Required:
Incubator, Laminar Flow

Material Required:
Sterile Nutrient Agar Plates, Sterile Cotton Swabs, Micropipette (Capacity 100 μl-1000 μl), 16 hr. old culture of following strains (Appr. density is 10⁸ CFU/ml) Escherichia coli (MDR), Pseudomonas aeruginosa (MDR), Methicillin Resistant Staphylococcus aureus.

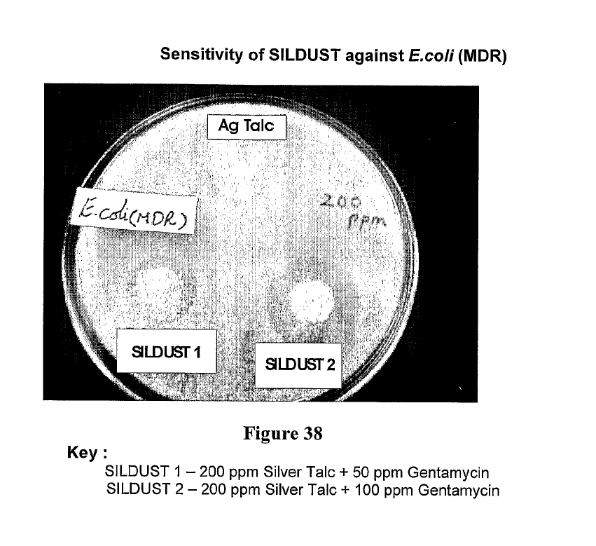

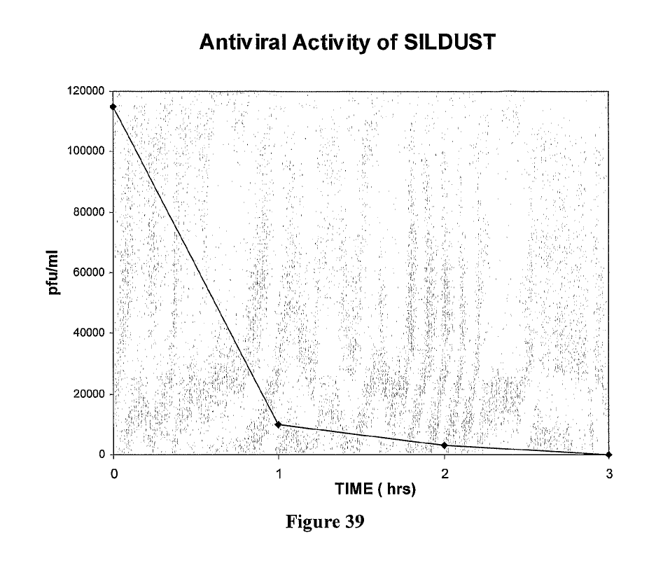

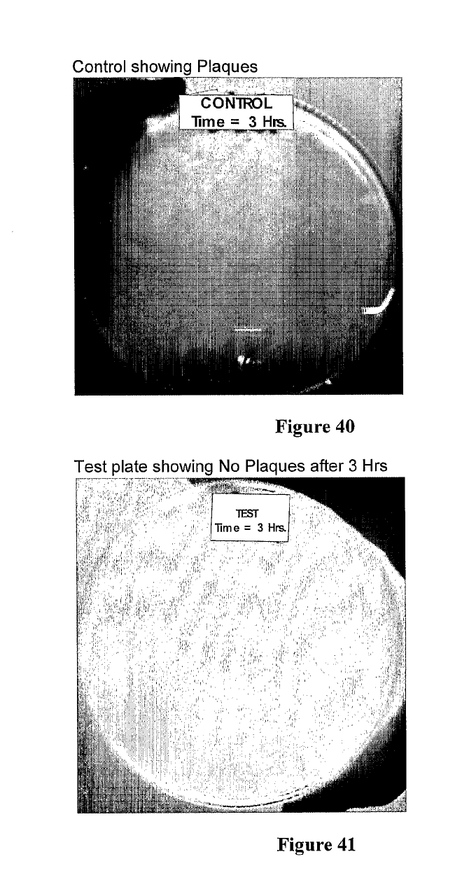

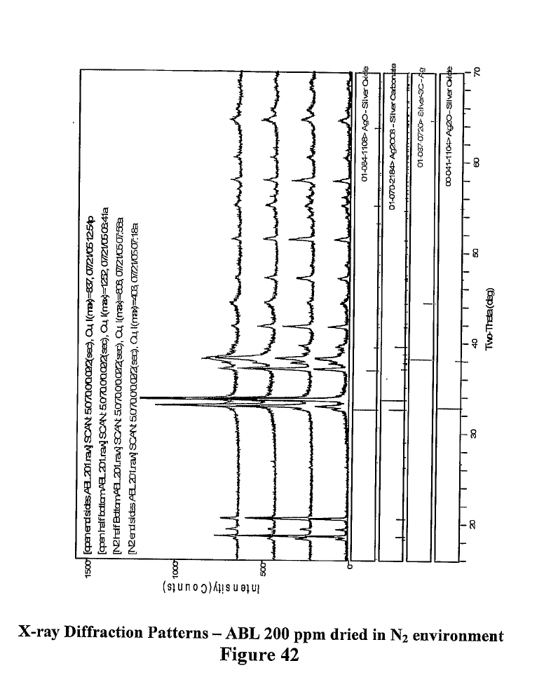

Method:
Surface spread 0.1 ml of culture onto nutrient agar surface using sterile cotton swabs. Keep aside for 15 minutes.
After 15 minutes aseptically bore wells into the agar surface with the help of a 10 mm cork borer.
Introduce 10 mg of SILDUST (200 ppm Silver talc+100 ppm Gentamycin) into one well.
Introduce 100 μl of 100 ppm Gentamycin to another well. Also introduce 200 ppm of Silver talc+100 μl of distilled water. Both serving as controls.
Incubate the plates at about 37° C. for about 24 hours and observe.
Measure the zone of inhibition in mm using HiMedia zone, reader.
Results: As given in Table 28 below and in FIG. 38.

TABLE 28
SILDUST Sensitivity

| | Zone of Inhibition | | |
|---|---|---|---|
| | 100 ppm | 200 ppm | |
| Culture | Gentamycin | ASAPTalc | SILDUST* |
| Escherichia coli | 24 mm | 17 mm | 26 mm |
| (MDR) | | | |

SILDUST* → 200 ppm ASAP Talc + 100 ppm Gentamycin

Conclusion: There is a synergistic activity observed for SILDUST (containing 200 ppm Silver Talc and 100 ppm Gentamycin).

Key:

SILDUST 1—200 ppm Silver Talc+50 ppm Gentamycin

SILDUST 2—200 ppm Silver Talc+100 ppm Gentamycin

SILDUST—Antibacterial Activity

Goal: To determine killing time of SILDUST against microorganisms.

Procedure:

Equipment Required:
Incubator, Laminar Flow, Weighing balance.

Material Required:
Sterile Phenol Red Dextrose Broth, Micropipette, 16 hr. old culture of following strains (Approximate density is 10⁸ CFU/ml) Escherichia coli (MDR), Pseudomonas aeruginosa (MDR), Methicillin Resistant Staphylococcus aureus.

Method:
Prepare 5 ml aliquot containing 2 g of SILDUST in Sterile test tube.
Inoculate 0.1 ml culture in the above solution. Vortex thoroughly.
At time intervals 0, 5, 10 . . . 50 minutes inoculate loopful of the sample under test into 5 ml of Sterile Phenol Red Dextrose Broth. Vortex thoroughly.
Incubate at about 37° C. for about 24 hours.
Observe for growth.
For negative control, loopful of uninoculated SILDUST was suspended in 5 ml Sterile Phenol Red Dextrose Broth and incubated at about 37° C. for about 24 hours.
For positive control, loopful of culture under test was inoculated in 5 ml Sterile Phenol Red Dextrose Broth and incubated at about 37° C. for about 24 hours.

Results: See Tables 29, 30, and 31

TABLE 29
Escherichia coli (MDR)

| Time Interval | 100 ppm | 200 ppm | | |
|---|---|---|---|---|
| (minutes) | Gentamycin | ASAPTalc | SILDUST* | Wokadine* |
| 0 | + | + | + | − |
| 5 | + | + | + | − |
| 10 | + | + | + | − |
| 15 | + | + | + | − |
| 20 | + | + | + | − |
| 25 | + | + | + | − |
| 30 | + | + | + | − |
| 35 | + | + | + | − |
| 40 | + | + | + | − |
| 45 | + | + | + | − |
| 50 | + | + | − | − |
| Positive | + | + | + | + |
| Control | | | | |
| Negative | − | − | − | − |
| Control | | | | |

SILDUST* → 200 ppm ASAP Talc + 100 ppm Gentamycin
Wokadine* → 200 ppm of available Iodine
Key:
+ → Growth
− → No Growth

Conclusion: Combination shows synergistic activity. The tube with WOKADINE (discussed below at end of Example) turned brown within a few seconds of powder addition to media due to iodine release. Though WOKADINE shows a faster kill, such high Cytotoxicity is undesirable for woundhealing.

TABLE 30
Pseudomonas aeruginosa (MDR)

| Time Interval | 100 ppm | 200 ppm | | |
|---|---|---|---|---|
| (minutes) | Gentamycin | ASAPTalc | SILDUST* | Wokadine* |
| 0 | + | + | + | − |
| 5 | + | + | + | − |
| 10 | + | + | + | − |
| 15 | + | + | + | − |
| 20 | + | + | + | − |
| 25 | + | + | + | − |
| 30 | + | + | + | − |
| 35 | + | + | + | − |
| 40 | + | + | − | − |
| 45 | + | + | − | − |
| 50 | − | − | − | − |
| Positive | + | + | + | + |
| Control | | | | |
| Negative | − | − | − | − |
| Control | | | | |

SILDUST* → 200 ppm ASAP Talc + 100 ppm Gentamycin
Wokadine* → 200 ppm of available Iodine
Key:
+ → Growth
− → No Growth

Conclusion: Combination shows synergistic activity.

TABLE 31
MRSA

| Time Interval | 100 ppm | 200 ppm | | |
|---|---|---|---|---|
| (minutes) | Gentamycin | ASAP Talc | SILDUST* | Wokadine* |
| 0 | + | + | + | − |
| 5 | + | + | + | − |
| 10 | + | + | − | − |
| 15 | − | + | − | − |
| 20 | − | + | − | − |
| 25 | − | + | − | − |
| 30 | − | − | − | − |
| 35 | − | − | − | − |
| 40 | − | − | − | − |
| 45 | − | − | − | − |
| 50 | − | − | − | − |
| Positive | + | + | + | + |
| Control | | | | |
| Negative | − | − | − | − |
| Control | | | | |

SILDUST* → 200 ppm ASAP Talc + 100 ppm Gentamycin
Wokadine* → 200 ppm of available Iodine
Key:
+ → Growth
− → No Growth

Conclusion: Combination shows synergistic activity.

SILDUST—Antibacterial Activity

Goal: To determine sensitivity of bacteriophage host to SILDUST.

Principle: A suitable dilution has to be arrived at to remove a false positive test due to host kill by SILDUST .

Procedure:

Principle:
T even bacteriophage and Escherichia coli host are used as detection system. Silver concentration in SILDUST has to be neutralized through dilution so as not to kill the host. Experimental aliquots were prepared as follows;
   1. Test—Phage+SILDUST
   2. Control—Phage+Saline

Equipment Required:
Weighing Balance, Laminar Airflow Unit, Incubator.

Material Required:
Petri Plates, Marker, Spatula, Micropipette.

Method:
Prepare 2.5 ml aliquot containing about 1 gm of SILDUST (which shows no bactericidal effect) and Saline in separate Sterile test tubes.
To each add about 0.1 ml of phage lysate (approx. 10¹⁰ infectious phage particles per ml).
Mix properly on vortex mixer and incubate at about 37° C.
Att =0, 1 and hourly intervals thereafter withdraw 0.5 ml aliquots and dilute to the pilot dilution of SILDUST which shows no bactericidal effect.
Spot this dilution on a confluent freshly prepared host lawn. This has to be done for test as well as controls.
Incubate the plate at about 37° C. for about 24 hours.
Mix 0.1 ml of this dilution with 0.5 ml of exponentially growing host and incubate at about 37° C. for about 15 minutes.
Add 7 ml of molten soft agar to it.
Vortex thoroughly and overlay on a St. Nutrient agar plate.
Incubate the plate at about 37° C. for about 24 hours.
Check for plaques on the lawn and enumerate plaque forming units on the overlay.

Results: See Table 32

TABLE 32
SILDUST Pilot

| Dilutions | Result |
|---|---|
| 10⁻¹ | + |
| 10⁻² | − |
| 10⁻³ | − |
| 10⁻⁴ | − |

Key:
+ → Presence of active phage particles.
− → Absence of Phage particles.

SILDUST—Antiviral Activity

: To determine antiviral activity of SILDUST using a bacteriophage detection system.

Procedure: Same as “SILDUST—ANTIBACTERIAL ACTVITY, Part 2”

Results: As per Table 33 and 34

TABLE 33
Kill Time of SILDUST

| Time Intervals | | |
|---|---|---|
| (hrs.) | Saline | SILDUST* |
| 0 | + | + |
| 1 | + | + |
| 2 | + | − |
| 3 | + | − |

SILDUST* → 200 ppm ASAP Talc + 100 ppm Gentamycin
Key:
+ → Presence of active phage particles.
− → Absence of Phage particles.

TABLE 34
Phage Enumeration

| Time Intervals | Saline | |
|---|---|---|
| (hrs.) | (pfu/ml) | SILDUST* (pfu/ml) |
| 0 | TNTC | 1.15 × 10⁵ |
| 1 | TNTC | 1.0 × 10⁴ |
| 2 | TNTC | 3.0 × 10³ |
| 3 | TNTC | Nil |

SILDUST* → 200 ppm ASAP Talc + 100 ppm Gentamycin
Key:
TNTC → Too Numerous To Count.
pfu/ml - Titre of infectious phage particles

Conclusion: SILDUST was found to show no bactericidal activity against host culture at 10⁻² dilution. The antiviral activity was checked at the same dilution of SILDUST and found to be effective. The plaque forming units were found to decrease from 10⁵ to zero in 3 hrs. proving that SILDUST could probably have activity against animal viruses too.

The following composition was used in the experiments immediately above herein:

Media Composition

Nutrient Agar:

| Peptone | 10.0 gm |
|---|---|
| Sodium chloride | 5.0 gm |
| Meat extract | 3.0 gm |
| Distilled water | 900 ml |
| Agar | 2.5 gm |
| pH | 7.2 ± 0.2 |

Phenol Red Dextrose Broth

| Proteose Peptone | 10.00 g/lt |
|---|---|
| Beef Extract | 1.00 g/lt |
| Sodium chloride | 5.0 g/lt |
| Dextrose | 5.0 g/lt |
| Phenol Red | 0.018 g/lt |
| pH | 7.4 ± 0.2 |

Soft Agar:

| | |
|---|---|
| Agar | 1.0% |

Saline:

| | |
|---|---|
| Sodium Chloride | 0.9% |

### Wokadine

Mfg. Lic. No.: AD/200-A

Batch No.: WNR 5008

Mfg. Date: March 2005

Expiry Date: March 2008

Active Ingredients:

Povidone Iodine IP 5% w/w

Mfgd. By:

Navketan Research and Lab. Ltd.

Additions of Silver/Water to Povidone Iodine 10% Solution

Another example of an additive that works favorably with the silver/water compositions of the present invention is Povidone iodine. Iodine is a well known prophylaxis in medicine for treatment against a wide range of pathogens. Iodine is commercially available in various concentrations, but a commonly used, and preferred, concentration is 10%. In this preferred embodiment of the invention, a synergistic combination comprises about 25-50% by volume substitution of the silver/water mixture replacing the 10% iodine solution. While some reactions between the silver/water mixture and iodine are possible, it appears from the experimental results that the synergistic combination of the silver/water with povidone iodine may function as a topical disinfectant (e.g., an ointment) and/or as prophylaxis against infection in cuts, burns and or scrapes, etc.

Specifically, the synergistic activity of 32 ppm silver/water compositions combined with varying percentages of Povidone Iodine (PI) was investigated against numerous bacteria. The methods of testing and results follow. It can be concluded from these results that by adding these two materials together, a synergistic relationship exists. This synergism can be utilized to result in an excellent topical disinfectant.

The following claims are thus to be understood to include what is specifically illustrated and described above, what is conceptually equivalent, what can be obviously substituted and also what essentially incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

We claim:

1. A composition of silver in water comprising a total concentration of silver of between about 5 and 40 parts per million, said silver in the form of silver nanoparticles having an interior of elemental silver and a surface of at least one silver oxide, wherein a majority of the silver particles have a maximum diameter less than 0.015 micrometers, wherein a majority of the colloidal silver particles have a minimum diameter greater than 0.005 micrometers, and wherein the composition manifests antimicrobial properties.

2. The composition according to claim 1, further comprising hydrogen peroxide.

3. The composition according to claim 2, wherein the hydrogen peroxide concentration is between about 1% wght/v and about 3.0% wght/v.

4. The composition according to claim 1, further comprising EDTA.

5. The composition according to claim 4, wherein said EDTA comprises disodium EDTA.

6. The composition according to claim 1, wherein the composition comprises hydrogel formed by dissolving a hydrophilic polymer into the composition of silver in water.

7. The composition according to claim 6 formulated as an amorphous gel.

8. The composition according to claim 6 formulated as a solid gel sheet.

9. The composition according to claim 8, wherein the hydrophilic polymer is selected from the group consisting of gelatin, carbohydrate polymers and acrylic acid copolymers.

10. The composition according to claim 9, wherein the carbohydrate polymer comprises at least one polymer selected from the group consisting of cellulose derivatives, alginate, carrageenan, and plant gums.

11. A method of treating a disease selected from the group consisting of malaria, fungal infections of the skin, bacterial infections of the skin, vaginal infections, urinary tract infections, tonsillitis, pelvic inflammatory disease, pharyngitis, gonorrhea, conjunctivitis, otitis, respiratory tract infections, and nasal infections, comprising the step of administering an aliquot of the composition according to claim 1 to a person afflicted with the disease.

12. A method of treating a disease selected from the group consisting of malaria, fungal infections of the skin, bacterial infections of the skin, vaginal infections, urinary tract infections, tonsillitis, pelvic inflammatory disease, pharyngitis, gonorrhea, conjunctivitis, otitis, respiratory tract infections, and nasal infections, comprising the step of administering silver EDTA.

13. A method for eliminating microbes selected from the group consisting of Bacillus anthracis, Bacillus subtilis, Candida albicans, Mycobacteria bovis, Mycobacteria tuberculosis, Pseudomonas aeruginosa, Salmonella choleraesius, Staphylococcus aureus, Trichomonas vaginalis, and Yersinia pestis comprising exposing said microbes to silver EDTA.

14. The method of claim 13, wherein said exposing comprises ingesting silver EDTA.

15. A method for eliminating microbes selected from the group consisting of Bacillus anthracis, Bacillus subtilis, Candida albicans, Mycobacteria bovis, Mycobacteria tuberculosis, Pseudomonas aeruginosa, Salmonella choleraesius, Staphylococcus aureus, Trichomonas vaginalis, and Yersinia pestis comprising exposing said microbes to at least one composition selected from the group consisting of silver EDTA, silver EDDS, silver curcuminate, silver berberine, and silver tetracycline.

16. A method for delivering at least one metal to a biologic organism comprising attaching at least one metal selected from the group of metals consisting of silver, copper, zinc, platinum, titanium, and mixtures and alloys thereof to at least one clathrate to form a metal/clathrate structure, and exposing said biologic organism to said metal/clathrate structure.

17. The method of claim 16, wherein said clathrate comprises at least one kaolinite.

18. The method of claim 16, wherein said clathrate comprises at least one zeolite.

19. The method of claim 16, wherein said at least one metal comprises silver.

20. A prophylactic treatment for livestock comprising adding AgEDTA to at least one of the livestock feed and livestock water.

21. A prophylactic treatment for humans and animals comprising adding AgEDTA to anything that said human or animal ingests.

22. The method of claim 21, wherein said AgEDTA is added in an amount sufficient to prevent infections.

23. The method of claim 21, wherein said AgEDTA is added to the composition of claim 1 in an amount less than 20 ppm.

24. A method for treatment of human and animal infections comprising ingesting Ag EDTA in a quantity sufficient to ameliorate said infection.

25. A method for treatment of human or animal infection comprising ingesting at least one member selected from the group consisting of AgEDTA, silver EDDS, silver curcuminate, silver berberine, and silver tetracycline.

26. A method for treatment of human or animal skin surfaces comprising forming a paste or gel from at least one member selected from the group consisting of AgEDTA, silver EDDS, silver curcuminate, silver berberine, and silver tetracycline and contacting said paste or gel with said human or animal skin surface.

27. A gel or paste product comprising at least one member selected from the group consisting of AgEDTA, silver EDDS, silver curcuminate, silver berberine, and silver tetracycline.

28. A method of enhancing antibiotic dose efficacy comprising adding to said antibiotic dose at least one material selected from the group consisting of EDTA and AgEDTA.

29. The method of claim 28, wherein AgEDTA is added to said antibiotic dose.

30. The method of claim 11, further comprising adding a selected antibiotic dose, said selected antibiotic dose being based on antibiotics having at least some known efficacy against said disease.

31. The composition of claim 1, further comprising at least one material selected from the group consisting of AgEDTA, silver EDDS, silver curcuminate, silver berberine, and silver tetracycline.
